SEQUENCE LISTING (1213 sequences)

SEQ ID NO: 1 - DNA, 26 bp, Artificial
  Synthetic
aaattatutctagcccaaaaaaacgg

SEQ ID NO: 2 - DNA, 24 bp, Artificial
  Synthetic
actggccgucgttttacaacgtcg

SEQ ID NO: 3 - DNA, 20 bp, Artificial
  Synthetic
agattagcggatcctacctg

SEQ ID NO: 4 - DNA, 21 bp, Artificial
  Synthetic
gctgcaaggcgattaagttgg

SEQ ID NO: 5 - DNA, 38 bp, Artificial
  Synthetic
acggccagutgatacgattaggacaatggtcaccgacg

SEQ ID NO: 6 - DNA, 30 bp, Artificial
  Synthetic
agtccgaucccaactattttgtccgcccac

SEQ ID NO: 7 - DNA, 24 bp, Artificial
  Synthetic
aataattutccggggcctgctctc

SEQ ID NO: 8 - DNA, 35 bp, Artificial
  Synthetic
aataattuggagtatatatgcaactacataacgcc

SEQ ID NO: 9 - DNA, 32 bp, Artificial
  Synthetic
aataattuattgcaggtaagatggcatctatg

SEQ ID NO: 10 - DNA, 344 bp, Artificial
  Synthetic
agtccgatcccaactattttgtccgcccactggtgttgctggactacctgtccaacatcttccgggttggcggtgaagaccagttatccaagtaccggaagaatatcggtcaggcatagggactagctccaatggaatgagccgccaggacggcaaaccgaagccccgccatcgtgcggggcttctttttttccagtcagcaaacttgaaatacacccttaagggtgtattattgtttccacggaaggcgaatcgccaacccctcactgccgtataggcagcccagaatgtacggagatcaccacacgtcggtgaccattgtcctaatcgtatcaactggccgt

SEQ ID NO: 11 - DNA, 423 bp, Artificial
  Synthetic
agtccgatcccaactattttgtccgcccacgagacaaggtcgccttgtctcgacaacgcccccgcgaaaaccgcccctgtgagccgtccgccaccagacccgccaccgtcctaccgcccaaaccgccccgcgcgcgtctacccgcgttagacccgcgttagattcgatttccggggcctgctctcgccgcccccttcggcttcgctcagggcaggcttgcgaccgtcgcccgattgcgcgacagtagagacagggcgccgccttgtctgatgctctcgttcactgccggataggcagccaaggaaatcatgctcggccacggacggtcttccccctgcgcccccgcaacctattgtgcacatcgcccgccgtcgatcatggcggccgtcggtgaccattgtcctaatcgtatcaactggccgt

SEQ ID NO: 12 - DNA, 396 bp, Artificial
  Synthetic
agtccgatcccaactattttgtccgcccaccacagcccttagcatttccatgctatacaagtcgcaccttaagcagtctggcaaactgccttttgggattaccagtcccaaaccttgaacgcctctaaggatttaaaaccttaggggtgtttattttatctgggagtatatatgcaactacataacgccatatttgttgatggtcgcatcacagcaaatagcagtctaacgaccttttaaatttctactgtttgtagatttcctgttggggattatcagcggcttattggttatttgcgctttgacagattttgtgtgaaatatcctgacagcaaggcggattttaactgctttgagccgtcggtgaccattgtcctaatcgtatcaactggccgt

SEQ ID NO: 13 - DNA, 438 bp, Artificial
  Synthetic
agtccgatcccaactattttgtccgcccacatgctacatcgttatgcatcagtattgatggatgcaggttttagctttgaaagtattaaagagaaaactattgctttaaataataagttaatagataaacttgatgaactggagttagctaatacaatattccataccattgcaggtaagatggcatctatgggcagaatgtagcttttaaccataacatcgcattctgcggtgttatggttttgtttttaaaccatgtcaattgtctaacaactttttaaatttctactgtttgtagattagattagataaccatgtcaattaatcataggagactttatgagtcaagttaatgaccatttagtacttatctgtggtgaatctagtacaggtaaatcagcgtcggtgaccattgtcctaatcgtatcaactggccgt

SEQ ID NO: 14 - DNA, 42 bp, Artificial
  Synthetic
gttcactgccgtataggcagctaagaaaaacggccgacgctt

SEQ ID NO: 15 - DNA, 44 bp, Artificial
  Synthetic
cggccgtttttcttagctgcctatacggcagtgaacaaattatt

SEQ ID NO: 16 - DNA, 35 bp, Artificial
  Synthetic
tctcgttcactgccggataggcagccaaggaaatc

SEQ ID NO: 17 - DNA, 35 bp, Artificial
  Synthetic
cagtgatttccttggctgcctatccggcagtgaac

SEQ ID NO: 18 - DNA, 36 bp, Artificial
  Synthetic
gtctaacgaccttttaaatttctactgtttgtagat

SEQ ID NO: 19 - DNA, 44 bp, Artificial
  Synthetic
atctacaaacagtagaaatttaaaaggtcgttagacaaattatt

SEQ ID NO: 20 - DNA, 24 bp, Artificial
  Synthetic
gataatctcttatagaattgaaag

SEQ ID NO: 21 - DNA, 36 bp, Artificial
  Synthetic
gtgctcaacgcctaacggcatcaaagttatattcag

SEQ ID NO: 22 - DNA, 25 bp, Artificial
  Synthetic
ctttcaattctctttgagttgcaac

SEQ ID NO: 23 - DNA, 37 bp, Artificial
  Synthetic
gcttcacatccttcgatccgataaaggatactgaaag

SEQ ID NO: 24 - DNA, 25 bp, Artificial
  Synthetic
gattaatcctaaaaggaattgaaag

SEQ ID NO: 25 - DNA, 25 bp, Artificial
  Synthetic
gcatatccctaaagggaattgaaag

SEQ ID NO: 26 - DNA, 37 bp, Artificial
  Synthetic
ctttcagtaccctccaagcgggtcgagtcgctgcaac

SEQ ID NO: 27 - DNA, 37 bp, Artificial
  Synthetic
cttgcagtcccctcctacggggcgatggccctgaaac

SEQ ID NO: 28 - DNA, 36 bp, Artificial
  Synthetic
gtgctcaacgcctttcggcatcatcggaattctcac

SEQ ID NO: 29 - DNA, 30 bp, Artificial
  Synthetic
gtttcaattccacactggttcgattaaaag

SEQ ID NO: 30 - DNA, 30 bp, Artificial
  Synthetic
atttcaattccactatggtgcgattagaag

SEQ ID NO: 31 - DNA, 30 bp, Artificial
  Synthetic
ctttcaattctcctagagtcttattgcaac

SEQ ID NO: 32 - DNA, 37 bp, Artificial
  Synthetic
cttgcagtaccctcagacgggtcgaacccaatgcaac

SEQ ID NO: 33 - DNA, 24 bp, Artificial
  Synthetic
gttaatctactatagaattgaaag

SEQ ID NO: 34 - DNA, 30 bp, Artificial
  Synthetic
gtttcaattccttataggtacagtgaaaac

SEQ ID NO: 35 - DNA, 37 bp, Artificial
  Synthetic
gttgcaccgatctccccgatgaggaggggactgaaac

SEQ ID NO: 36 - DNA, 25 bp, Artificial
  Synthetic
ctttcaattctttctgagttgcatc

SEQ ID NO: 37 - DNA, 25 bp, Artificial
  Synthetic
gaatctccctaaagggagtagaaag

SEQ ID NO: 38 - DNA, 24 bp, Artificial
  Synthetic
gcatctcaaaaagaggattgaaag

SEQ ID NO: 39 - DNA, 25 bp, Artificial
  Synthetic
ccttctattctctttagagaatatc

SEQ ID NO: 40 - DNA, 24 bp, Artificial
  Synthetic
gatgatctactatagaattgaaag

SEQ ID NO: 41 - DNA, 37 bp, Artificial
  Synthetic
gtttcaatccttgttttaatggatcttgctctcgaat

SEQ ID NO: 42 - DNA, 30 bp, Artificial
  Synthetic
atttacattccaatctggttagattacaat

SEQ ID NO: 43 - DNA, 29 bp, Artificial
  Synthetic
atttacatttcataataagtagttaaaac

SEQ ID NO: 44 - DNA, 30 bp, Artificial
  Synthetic
atttcaatcccaatatggtctgattttaac

SEQ ID NO: 45 - DNA, 30 bp, Artificial
  Synthetic
gtttcaattccttataggtactttataaac

SEQ ID NO: 46 - DNA, 30 bp, Artificial
  Synthetic
gttaaaaagtaactatattagtttttaaat

SEQ ID NO: 47 - DNA, 30 bp, Artificial
  Synthetic
gtattaatagtagcaaagtgtaatgtaaat

SEQ ID NO: 48 - DNA, 30 bp, Artificial
  Synthetic
atttaagtagcaccatattggaatgtaaat

SEQ ID NO: 49 - DNA, 30 bp, Artificial
  Synthetic
atttcaatacatctaatgttactattcaac

SEQ ID NO: 50 - DNA, 29 bp, Artificial
  Synthetic
gtattaattgtaccttatggaattgaaat

SEQ ID NO: 51 - DNA, 37 bp, Artificial
  Synthetic
cttccagagtaacatccactagaataaggattgaaac

SEQ ID NO: 52 - DNA, 30 bp, Artificial
  Synthetic
gtattaatttaaatatattagaatgtaaat

SEQ ID NO: 53 - DNA, 30 bp, Artificial
  Synthetic
gtttcaaactccaacggagtaaattctaat

SEQ ID NO: 54 - DNA, 30 bp, Artificial
  Synthetic
atttcaatacatcaaatgtgttggttccgc

SEQ ID NO: 55 - DNA, 30 bp, Artificial
  Synthetic
atttcaaaacttttcaagtttatagttcat

SEQ ID NO: 56 - DNA, 30 bp, Artificial
  Synthetic
gtcctcatcgcccctacgaggggtagcaac

SEQ ID NO: 57 - DNA, 30 bp, Artificial
  Synthetic
gttaaacaataacatgagatgttttgaaat

SEQ ID NO: 58 - DNA, 37 bp, Artificial
  Synthetic
ctttgaattaattgtccaacagaacaaggattgaaac

SEQ ID NO: 59 - DNA, 30 bp, Artificial
  Synthetic
gtttcagacgaacccttgtggggttgaagc

SEQ ID NO: 60 - DNA, 30 bp, Artificial
  Synthetic
gtttgtagcctgcctatgagggattgaaac

SEQ ID NO: 61 - DNA, 30 bp, Artificial
  Synthetic
gtttcaattccaatatggttcgattaaaag

SEQ ID NO: 62 - DNA, 29 bp, Artificial
  Synthetic
atttccatactattgagtctattttaaac

SEQ ID NO: 63 - DNA, 30 bp, Artificial
  Synthetic
gtttcaattgtaccatacaggaattgaaac

SEQ ID NO: 64 - DNA, 30 bp, Artificial
  Synthetic
ctagaaccgacacaaaagttgtattgaaac

SEQ ID NO: 65 - DNA, 30 bp, Artificial
  Synthetic
ctataaatctatccatattggattttaaat

SEQ ID NO: 66 - DNA, 30 bp, Artificial
  Synthetic
atttcaatcctattatagtctgattttaac

SEQ ID NO: 67 - DNA, 30 bp, Artificial
  Synthetic
gtttccatcccttataggaagtttaaaaac

SEQ ID NO: 68 - DNA, 31 bp, Artificial
  Synthetic
agtttcaatacaccatgtgttactgtcaaac

SEQ ID NO: 69 - DNA, 30 bp, Artificial
  Synthetic
gtttcaattctaccttagttcaattaaaag

SEQ ID NO: 70 - DNA, 29 bp, Artificial
  Synthetic
ctttatatcccactacgttcagataaaac

SEQ ID NO: 71 - DNA, 30 bp, Artificial
  Synthetic
gttaaaatcagactattttaggattgaaat

SEQ ID NO: 72 - DNA, 30 bp, Artificial
  Synthetic
gtttttagcctacctatgaggaattgaaac

SEQ ID NO: 73 - DNA, 30 bp, Artificial
  Synthetic
atttacatttctcttaagttaaataaaaac

SEQ ID NO: 74 - DNA, 30 bp, Artificial
  Synthetic
atttcaattccactatggttcgattaaaag

SEQ ID NO: 75 - DNA, 30 bp, Artificial
  Synthetic
attaaagtagaaccatattggaatgtaaag

SEQ ID NO: 76 - DNA, 29 bp, Artificial
  Synthetic
gtcgtcatcagccctggagggttcgcaac

SEQ ID NO: 77 - DNA, 30 bp, Artificial
  Synthetic
gtttgctaatgacaatatttgtgttgaaac

SEQ ID NO: 78 - DNA, 30 bp, Artificial
  Synthetic
atcttaatcgtaccagagtggaattgaaat

SEQ ID NO: 79 - DNA, 37 bp, Artificial
  Synthetic
gtttcaatccttcttatcatggatggtggtcagtaag

SEQ ID NO: 80 - DNA, 30 bp, Artificial
  Synthetic
gattaactttaacatgagatgtatttaaat

SEQ ID NO: 81 - DNA, 30 bp, Artificial
  Synthetic
gtttgtgacccgcatataggggattgaaac

SEQ ID NO: 82 - DNA, 30 bp, Artificial
  Synthetic
atttaaattctaatatagaaatacataaat

SEQ ID NO: 83 - DNA, 30 bp, Artificial
  Synthetic
gtttgtatcgtacctatgaggaattgaaac

SEQ ID NO: 84 - DNA, 30 bp, Artificial
  Synthetic
ctcataatcgaagcagactgaaatttaaat

SEQ ID NO: 85 - DNA, 30 bp, Artificial
  Synthetic
cttaaagtattatcatattggaatttaaat

SEQ ID NO: 86 - DNA, 30 bp, Artificial
  Synthetic
gataaacaataacaagagttgtattgaaat

SEQ ID NO: 87 - DNA, 30 bp, Artificial
  Synthetic
attagagtattactaaagtagaatgtaaat

SEQ ID NO: 88 - DNA, 30 bp, Artificial
  Synthetic
gttgcaataagactctaggagaattgaaag

SEQ ID NO: 89 - DNA, 30 bp, Artificial
  Synthetic
atttacattctactgtagttctattaaagg

SEQ ID NO: 90 - DNA, 30 bp, Artificial
  Synthetic
gttttgatcgtacctatgagggattgaaac

SEQ ID NO: 91 - DNA, 30 bp, Artificial
  Synthetic
gttataatcgtactaatgtagaattgaaac

SEQ ID NO: 92 - DNA, 30 bp, Artificial
  Synthetic
cttgaaccaaaacatatgatgtattgaaat

SEQ ID NO: 93 - DNA, 30 bp, Artificial
  Synthetic
atttacattccactctttttagatttaaac

SEQ ID NO: 94 - DNA, 30 bp, Artificial
  Synthetic
gttgttagcttacctataaggaattgaaac

SEQ ID NO: 95 - DNA, 30 bp, Artificial
  Synthetic
gttgaaccttaacaagggttgtatttaaat

SEQ ID NO: 96 - DNA, 30 bp, Artificial
  Synthetic
cttttaatcgcaccatataggaattgaaat

SEQ ID NO: 97 - DNA, 29 bp, Artificial
  Synthetic
atttacataccacttagttaatataaaac

SEQ ID NO: 98 - DNA, 30 bp, Artificial
  Synthetic
gtattaatagatacattgtggaatgtaaag

SEQ ID NO: 99 - DNA, 30 bp, Artificial
  Synthetic
atttatatttaacaagcattgtttttaaag

SEQ ID NO: 100 - DNA, 30 bp, Artificial
  Synthetic
gttacagacgaaccctagttgggttgaagc

SEQ ID NO: 101 - DNA, 30 bp, Artificial
  Synthetic
gttgaaatcggaccaatgtgggattgaaac

SEQ ID NO: 102 - DNA, 29 bp, Artificial
  Synthetic
gttctaatcgaactatatagaattgaaat

SEQ ID NO: 103 - DNA, 30 bp, Artificial
  Synthetic
ctttacattccactctggtacgattctaat

SEQ ID NO: 104 - DNA, 29 bp, Artificial
  Synthetic
atttataactaacttagtgtaatttaaac

SEQ ID NO: 105 - DNA, 30 bp, Artificial
  Synthetic
gtttttagtctacctatgaggaattgaaac

SEQ ID NO: 106 - DNA, 30 bp, Artificial
  Synthetic
atttaaatacatcctatgttaaggttcaac

SEQ ID NO: 107 - DNA, 30 bp, Artificial
  Synthetic
ctttgaatctaaccatattggaatgtaaat

SEQ ID NO: 108 - DNA, 30 bp, Artificial
  Synthetic
attcaaaaacaacttttgttactgttaatc

SEQ ID NO: 109 - DNA, 29 bp, Artificial
  Synthetic
cctttaattgtaccttatggaattgaaac

SEQ ID NO: 110 - DNA, 30 bp, Artificial
  Synthetic
gtttaaattccacactggttcaatgctcac

SEQ ID NO: 111 - DNA, 37 bp, Artificial
  Synthetic
gtttaaatcctaaatccattaaaacaaggattgaaac

SEQ ID NO: 112 - DNA, 30 bp, Artificial
  Synthetic
ctcccaatcgcaccatactggaattgaaat

SEQ ID NO: 113 - DNA, 30 bp, Artificial
  Synthetic
atttcaaaacatcccatgttaaagttaaac

SEQ ID NO: 114 - DNA, 30 bp, Artificial
  Synthetic
gtttgtagccttccctttggggattgaaac

SEQ ID NO: 115 - DNA, 37 bp, Artificial
  Synthetic
gtcgaagagccacatccacgaaaacaaggattgaaac

SEQ ID NO: 116 - DNA, 30 bp, Artificial
  Synthetic
atttcaaaacatttcatgtttacggttaat

SEQ ID NO: 117 - DNA, 37 bp, Artificial
  Synthetic
ctttcagtcccctctaacggggcaatggccctgcaac

SEQ ID NO: 118 - DNA, 29 bp, Artificial
  Synthetic
gtttttatcttcctaagaggaatatgaac

SEQ ID NO: 119 - DNA, 29 bp, Artificial
  Synthetic
ctttataactaacatagttctattaatac

SEQ ID NO: 120 - DNA, 29 bp, Artificial
  Synthetic
gttcaaattcctctatggtcgatggtcac

SEQ ID NO: 121 - DNA, 29 bp, Artificial
  Synthetic
gtatcaatcgcaccttcgggtattgaaat

SEQ ID NO: 122 - DNA, 29 bp, Artificial
  Synthetic
ctttaaatcccacaattgggggctaaaac

SEQ ID NO: 123 - DNA, 30 bp, Artificial
  Synthetic
gtttcaattcccaacgggatggaattctac

SEQ ID NO: 124 - DNA, 29 bp, Artificial
  Synthetic
gttttatctgaactatgagggatgtaaac

SEQ ID NO: 125 - DNA, 29 bp, Artificial
  Synthetic
ctttccacacaattctgttctacggaaac

SEQ ID NO: 126 - DNA, 37 bp, Artificial
  Synthetic
gtttcaatccttgttgttctggatatagctctgaaag

SEQ ID NO: 127 - DNA, 27 bp, Artificial
  Synthetic
ggggaattaatagatacatagtgggat

SEQ ID NO: 128 - DNA, 29 bp, Artificial
  Synthetic
gtttctaattaaccgtgtggagttgaaag

SEQ ID NO: 129 - DNA, 37 bp, Artificial
  Synthetic
gttaaagagtaagttccattaaaacaaggattaaaac

SEQ ID NO: 130 - DNA, 37 bp, Artificial
  Synthetic
gtttcaatgcttgttattttgcaagttactcttttag

SEQ ID NO: 131 - DNA, 37 bp, Artificial
  Synthetic
gtttcagtgctctaccgtgagccgaaattgttgaaag

SEQ ID NO: 132 - DNA, 30 bp, Artificial
  Synthetic
gttttaataatacctatgagggattgaaac

SEQ ID NO: 133 - DNA, 30 bp, Artificial
  Synthetic
gtttcaatccttattgggttcgattaggac

SEQ ID NO: 134 - DNA, 36 bp, Artificial
  Synthetic
gtgctcaacgcctttcggcatcagaggtttttgcac

SEQ ID NO: 135 - DNA, 30 bp, Artificial
  Synthetic
gttaaaatcagaccttagagggattgaaac

SEQ ID NO: 136 - DNA, 36 bp, Artificial
  Synthetic
ccgcaccgatctccgatgccgaaaggcgttgagcac

SEQ ID NO: 137 - DNA, 37 bp, Artificial
  Synthetic
gttttaatctccgttggagcgacgttggggtgggaag

SEQ ID NO: 138 - DNA, 37 bp, Artificial
  Synthetic
gtcaaaatgacaaatccatcataacaaggattgaaac

SEQ ID NO: 139 - DNA, 35 bp, Artificial
  Synthetic
attgcaatttaaaaatccctattagggattgaaac

SEQ ID NO: 140 - DNA, 36 bp, Artificial
  Synthetic
ctgaatataactttgatgccgttaggcgttgagcac

SEQ ID NO: 141 - DNA, 30 bp, Artificial
  Synthetic
atttcaattccactaaggttcaattataac

SEQ ID NO: 142 - DNA, 34 bp, Artificial
  Synthetic
atttcaaaacatcacgaccaagaggatactaaaa

SEQ ID NO: 143 - DNA, 30 bp, Artificial
  Synthetic
gtttcaaaatccataggatcaaattgaaac

SEQ ID NO: 144 - DNA, 34 bp, Artificial
  Synthetic
tagcaacgcctaacggcataaagccacgaatcac

SEQ ID NO: 145 - DNA, 36 bp, Artificial
  Synthetic
gtgctcaacgcctttcggcatcattggaatcctcac

SEQ ID NO: 146 - DNA, 36 bp, Artificial
  Synthetic
gtttcaatcctctacgaggctaacgaggtttgcaac

SEQ ID NO: 147 - DNA, 37 bp, Artificial
  Synthetic
cttccagatgcatccaaatagaaactaggattgaaac

SEQ ID NO: 148 - DNA, 36 bp, Artificial
  Synthetic
gtgctcaacgccttccggcatcacagcgggcgtcac

SEQ ID NO: 149 - DNA, 36 bp, Artificial
  Synthetic
gtgttttaaccttagatgccataaggcgttgatcac

SEQ ID NO: 150 - DNA, 30 bp, Artificial
  Synthetic
ctttacattccactatgtttctattaattc

SEQ ID NO: 151 - DNA, 32 bp, Artificial
  Synthetic
gtcgccccccgcgcgggggcgtggattgaaac

SEQ ID NO: 152 - DNA, 33 bp, Artificial
  Synthetic
atttcaatccacgctctccatgaggagagcgac

SEQ ID NO: 153 - DNA, 37 bp, Artificial
  Synthetic
gtagcgcccggcctcaaagccgggcgaggattgaaac

SEQ ID NO: 154 - DNA, 32 bp, Artificial
  Synthetic
atttcaatccacgcacccgcaaagggtgcgac

SEQ ID NO: 155 - DNA, 33 bp, Artificial
  Synthetic
atttcaatccacgcgccccttgcggggcgcgac

SEQ ID NO: 156 - DNA, 32 bp, Artificial
  Synthetic
atttaaatccactagcctcagggaggctagac

SEQ ID NO: 157 - DNA, 33 bp, Artificial
  Synthetic
atttcaacccactccgcccgcgaggacggagac

SEQ ID NO: 158 - DNA, 32 bp, Artificial
  Synthetic
gtttcaacacacagccgcccgaaggcggctgc

SEQ ID NO: 159 - DNA, 32 bp, Artificial
  Synthetic
atttcaactcacagcctcacgaagaggctgac

SEQ ID NO: 160 - DNA, 33 bp, Artificial
  Synthetic
gtcacacctcgcatgaggtgtgtgagtagaaat

SEQ ID NO: 161 - DNA, 33 bp, Artificial
  Synthetic
gtttcaattcacgcagcccaagcgggctgcgac

SEQ ID NO: 162 - DNA, 32 bp, Artificial
  Synthetic
gtcactcccttcgcgggagtgtggattgaaat

SEQ ID NO: 163 - DNA, 32 bp, Artificial
  Synthetic
gtttcaatccgcaggctcgcgagagcctgatc

SEQ ID NO: 164 - DNA, 32 bp, Artificial
  Synthetic
gtttcaatccacacacccacgtagggtgtgac

SEQ ID NO: 165 - DNA, 32 bp, Artificial
  Synthetic
gtcgtaccttatataggtacgtggattgaaac

SEQ ID NO: 166 - DNA, 32 bp, Artificial
  Synthetic
atttcaattcacgcctctctgcgagaggctac

SEQ ID NO: 167 - DNA, 33 bp, Artificial
  Synthetic
atttcaatccactagctctaaacagagctagac

SEQ ID NO: 168 - DNA, 33 bp, Artificial
  Synthetic
atttcaatccccgccttccgtgaggaaggcgac

SEQ ID NO: 169 - DNA, 32 bp, Artificial
  Synthetic
gtttcaattcacgcacccgcgaggggtgcgac

SEQ ID NO: 170 - DNA, 33 bp, Artificial
  Synthetic
gtcgcaccccacacgggttgcgtggattgaaat

SEQ ID NO: 171 - DNA, 37 bp, Artificial
  Synthetic
gattcgcccgccagaaatggcgggcgcggattgaaac

SEQ ID NO: 172 - DNA, 32 bp, Artificial
  Synthetic
gtcacttcccgtgcgggagtgtggattgaaat

SEQ ID NO: 173 - DNA, 32 bp, Artificial
  Synthetic
gtcaccctccgtgcgagggtgtggattgaaat

SEQ ID NO: 174 - DNA, 32 bp, Artificial
  Synthetic
gtttcaattcacggtcgcgcgtgaggcgaccg

SEQ ID NO: 175 - DNA, 33 bp, Artificial
  Synthetic
gtcactccccgcatggggagtgcgggttgaaat

SEQ ID NO: 176 - DNA, 32 bp, Artificial
  Synthetic
gtttcaattcgcgctcccgcgtggggagcgac

SEQ ID NO: 177 - DNA, 33 bp, Artificial
  Synthetic
atttcaatccacagccccatcgcagagactgac

SEQ ID NO: 178 - DNA, 32 bp, Artificial
  Synthetic
atttcaatccacaccactgcatcagtggtgac

SEQ ID NO: 179 - DNA, 32 bp, Artificial
  Synthetic
atttcaactcacacgctcacgtggagcgtgac

SEQ ID NO: 180 - DNA, 33 bp, Artificial
  Synthetic
atttcaatccacgcacctcgtgtgaggtgcgac

SEQ ID NO: 181 - DNA, 32 bp, Artificial
  Synthetic
gtttcgatccacaggctcttacgagcctgacc

SEQ ID NO: 182 - DNA, 31 bp, Artificial
  Synthetic
cagccacctacgcgtggctgtgaattgaaac

SEQ ID NO: 183 - DNA, 33 bp, Artificial
  Synthetic
gtcgaggtctgcgaaggcctcgtggattgaaat

SEQ ID NO: 184 - DNA, 33 bp, Artificial
  Synthetic
atttctactcacacgcccctcgcggggcgtgac

SEQ ID NO: 185 - DNA, 33 bp, Artificial
  Synthetic
atttcaatccacactctctatgtagagagtgac

SEQ ID NO: 186 - DNA, 32 bp, Artificial
  Synthetic
ttttcaactcacgacctctcgtggaggtcgac

SEQ ID NO: 187 - DNA, 32 bp, Artificial
  Synthetic
atttcaattcatgcccctgcatgaggggcagc

SEQ ID NO: 188 - DNA, 30 bp, Artificial
  Synthetic
atttcaactcacgccctcacggagggcgac

SEQ ID NO: 189 - DNA, 32 bp, Artificial
  Synthetic
cttaaatccaccccgcccatgcgggcggggac

SEQ ID NO: 190 - DNA, 32 bp, Artificial
  Synthetic
gtcgttccccacgtgggaacgtggattgaaat

SEQ ID NO: 191 - DNA, 33 bp, Artificial
  Synthetic
gtttcaactcgcgcgccccggggggggtgcgac

SEQ ID NO: 192 - DNA, 33 bp, Artificial
  Synthetic
gtcgcactccgcaaggagtgcgtggattgaaac

SEQ ID NO: 193 - DNA, 37 bp, Artificial
  Synthetic
gtttcaatcagcccccgagggtgaggtcgggggagac

SEQ ID NO: 194 - DNA, 32 bp, Artificial
  Synthetic
ccagctacgtacgcgtagctgtgagttgaaac

SEQ ID NO: 195 - DNA, 32 bp, Artificial
  Synthetic
gatgccccccacacgggggcacgacttgaaac

SEQ ID NO: 196 - DNA, 32 bp, Artificial
  Synthetic
gtcgcactctatgtgagtgcgtggattgaaat

SEQ ID NO: 197 - DNA, 32 bp, Artificial
  Synthetic
gtttcgatccgcgctcccgcgtggggagcgac

SEQ ID NO: 198 - DNA, 37 bp, Artificial
  Synthetic
gtttcaatccgcatcccgaccggaggtcggaagtaac

SEQ ID NO: 199 - DNA, 30 bp, Artificial
  Synthetic
atttcaattccacaaaggtgcgattaaaag

SEQ ID NO: 200 - DNA, 30 bp, Artificial
  Synthetic
atttcaattgcaccatacaggaattaaaac

SEQ ID NO: 201 - DNA, 30 bp, Artificial
  Synthetic
gtttcaattccacattggttcgattaaaag

SEQ ID NO: 202 - DNA, 29 bp, Artificial
  Synthetic
cccttaatcgcaccgtatggaattgaaat

SEQ ID NO: 203 - DNA, 37 bp, Artificial
  Synthetic
gtagcaccccccctcacgggggggtgaggattgaaat

SEQ ID NO: 204 - DNA, 37 bp, Artificial
  Synthetic
gtcgcacccggccgcaaggccgggtgaggattgaaac

SEQ ID NO: 205 - DNA, 37 bp, Artificial
  Synthetic
gtttcaatcctcacccggcgcgctgaccgggtgcaac

SEQ ID NO: 206 - DNA, 37 bp, Artificial
  Synthetic
gtggcggtcgccctccggggcggccgaggatcgcaac

SEQ ID NO: 207 - DNA, 32 bp, Artificial
  Synthetic
gtttcaatccacgctcccgcgaggggagcgac

SEQ ID NO: 208 - DNA, 37 bp, Artificial
  Synthetic
gtttcaacccaccccccgcttcgtcagcgaggggaac

SEQ ID NO: 209 - DNA, 32 bp, Artificial
  Synthetic
gtttcaatccacgcgcccgcgtggggcgcgac

SEQ ID NO: 210 - DNA, 37 bp, Artificial
  Synthetic
gtttcaatcctcgcccgccccgaaggacgggcgctac

SEQ ID NO: 211 - DNA, 33 bp, Artificial
  Synthetic
gtttcaatccactccccccgagcgaggggagat

SEQ ID NO: 212 - DNA, 37 bp, Artificial
  Synthetic
gttgcgatcctcaccggccccggagggccggtgccac

SEQ ID NO: 213 - DNA, 33 bp, Artificial
  Synthetic
gtttcaatccactcccgtatcaccagggggaac

SEQ ID NO: 214 - DNA, 32 bp, Artificial
  Synthetic
gtcgcacccctcgcgggtgcgtggattgaaac

SEQ ID NO: 215 - DNA, 33 bp, Artificial
  Synthetic
gtttcaatccacgccccccgcggggggcgcgac

SEQ ID NO: 216 - DNA, 33 bp, Artificial
  Synthetic
gtttctatccgcacgccccgggcggggcgcgac

SEQ ID NO: 217 - DNA, 32 bp, Artificial
  Synthetic
gtcgcccccttcgcgggggcgtggattgaaac

SEQ ID NO: 218 - DNA, 37 bp, Artificial
  Synthetic
gtttcaatcctcacccggctttatagccgggtgcaac

SEQ ID NO: 219 - DNA, 33 bp, Artificial
  Synthetic
gtcgctccccgcaaggagggcgtggattgaaac

SEQ ID NO: 220 - DNA, 37 bp, Artificial
  Synthetic
gttgcgatcctcgcccggcgcgatggccgggcgcagc

SEQ ID NO: 221 - DNA, 32 bp, Artificial
  Synthetic
atttcaatccacgctcccgtgaggggagcgac

SEQ ID NO: 222 - DNA, 37 bp, Artificial
  Synthetic
gcagcgccgggcctccgcgcccggcgaggatcgcaac

SEQ ID NO: 223 - DNA, 37 bp, Artificial
  Synthetic
gtttcaatcctcaattaccttaaatagtaattgtcac

SEQ ID NO: 224 - DNA, 32 bp, Artificial
  Synthetic
gtcgccccccacacgggggcgtggattgaaac

SEQ ID NO: 225 - DNA, 35 bp, Artificial
  Synthetic
atcgcacctcctccgggccgggtgaggattgaaac

SEQ ID NO: 226 - DNA, 37 bp, Artificial
  Synthetic
gtttcaatccacgcccgttattgctaacgggcgaatc

SEQ ID NO: 227 - DNA, 32 bp, Artificial
  Synthetic
atttcaattcacactctcgtaaagagagtgac

SEQ ID NO: 228 - DNA, 32 bp, Artificial
  Synthetic
gtcgcgccccgcgtgggcgcgtggattgaaac

SEQ ID NO: 229 - DNA, 31 bp, Artificial
  Synthetic
gtcgcgcccccacgggcgcgtggattgaaac

SEQ ID NO: 230 - DNA, 33 bp, Artificial
  Synthetic
atttctatccacgccctccgcgaggagggcgac

SEQ ID NO: 231 - DNA, 37 bp, Artificial
  Synthetic
gtttcaatcctcgcccagcccgttggccgggcgctac

SEQ ID NO: 232 - DNA, 32 bp, Artificial
  Synthetic
gtcaccttccaaagggaggtgtggattgaaat

SEQ ID NO: 233 - DNA, 37 bp, Artificial
  Synthetic
gtttcaatcctcaccggccctttcgggccggtgctac

SEQ ID NO: 234 - DNA, 33 bp, Artificial
  Synthetic
gtcgccccctgcgagggaggcgtggattgaaac

SEQ ID NO: 235 - DNA, 33 bp, Artificial
  Synthetic
atttcaatccacgccccccgcgaggggggcgac

SEQ ID NO: 236 - DNA, 35 bp, Artificial
  Synthetic
gtttcaatccacgcccgtcatcctgacgggcgagc

SEQ ID NO: 237 - DNA, 33 bp, Artificial
  Synthetic
cgcccgcccctacacgcgggcgcggattgaaac

SEQ ID NO: 238 - DNA, 33 bp, Artificial
  Synthetic
gttgcactccactcggagtgcatgaattgaaat

SEQ ID NO: 239 - DNA, 31 bp, Artificial
  Synthetic
gtttcaatccacgcgcctgtgaaggcgcgac

SEQ ID NO: 240 - DNA, 32 bp, Artificial
  Synthetic
atttcaatccacaccaccgtgtagggggtgac

SEQ ID NO: 241 - DNA, 32 bp, Artificial
  Synthetic
gtcgctcccctcgtgggagcgtggattgaaat

SEQ ID NO: 242 - DNA, 34 bp, Artificial
  Synthetic
tgtagttcccggttattacttggtatgttataat

SEQ ID NO: 243 - DNA, 32 bp, Artificial
  Synthetic
atttcaatccactcacccatgaagggtgagac

SEQ ID NO: 244 - DNA, 33 bp, Artificial
  Synthetic
gtttcaatccacgcactccgtgcagagtgcgac

SEQ ID NO: 245 - DNA, 37 bp, Artificial
  Synthetic
gtttcaatccctatcgggtttttttatccgttgcgac

SEQ ID NO: 246 - DNA, 37 bp, Artificial
  Synthetic
gtttcaatccctaatagggattaagattaattgcaac

SEQ ID NO: 247 - DNA, 28 bp, Artificial
  Synthetic
ctcacaatccctcatggggatagataac

SEQ ID NO: 248 - DNA, 28 bp, Artificial
  Synthetic
gttccgaatccccatgaggggttatgag

SEQ ID NO: 249 - DNA, 28 bp, Artificial
  Synthetic
gtcctctctccccatgaggggtcaggag

SEQ ID NO: 250 - DNA, 24 bp, Artificial
  Synthetic
gtaacaacgacaagaaactaaaac

SEQ ID NO: 251 - DNA, 37 bp, Artificial
  Synthetic
gtttcaatccctatcgggttttcttatccgttgcgac

SEQ ID NO: 252 - DNA, 37 bp, Artificial
  Synthetic
gtggcaaaggcattgagcgcgactgagcgattgaaag

SEQ ID NO: 253 - DNA, 37 bp, Artificial
  Synthetic
gcgaaaatccccaataatccctttgagggattgaaac

SEQ ID NO: 254 - DNA, 37 bp, Artificial
  Synthetic
gttgcagcgcacttgaaaacccgacagggattgaaac

SEQ ID NO: 255 - DNA, 37 bp, Artificial
  Synthetic
gtttcaatctctaacgagttttctaaccgattgcgat

SEQ ID NO: 256 - DNA, 37 bp, Artificial
  Synthetic
gtttcagtccccttgcggggattaggtaagtggaaac

SEQ ID NO: 257 - DNA, 37 bp, Artificial
  Synthetic
gttggagaggcaattgtcgcgcttgagcgattgaaac

SEQ ID NO: 258 - DNA, 37 bp, Artificial
  Synthetic
gttgaaattacctttaatccctatgagggattgaaac

SEQ ID NO: 259 - DNA, 37 bp, Artificial
  Synthetic
gtcggaatggcgctaagcccgaatgagggattgaaac

SEQ ID NO: 260 - DNA, 37 bp, Artificial
  Synthetic
gtttcaatccctgaaagggtttcaggttcgttgaagc

SEQ ID NO: 261 - DNA, 37 bp, Artificial
  Synthetic
ctttcaatcccgcactcgggcttatccatcttctgac

SEQ ID NO: 262 - DNA, 37 bp, Artificial
  Synthetic
gttgcaatccctcaatcgggctttggcctcttctgac

SEQ ID NO: 263 - DNA, 37 bp, Artificial
  Synthetic
gtttcaatcccgttctgggttttctgggtgtcgcgac

SEQ ID NO: 264 - DNA, 37 bp, Artificial
  Synthetic
gtttcaatccgccgggcggatttttggcttttggaac

SEQ ID NO: 265 - DNA, 37 bp, Artificial
  Synthetic
gtttcaatcccgcgaacgggtttcatttatttgaaag

SEQ ID NO: 266 - DNA, 37 bp, Artificial
  Synthetic
gttgcaacttttattaatccctattagggattgaaac

SEQ ID NO: 267 - DNA, 37 bp, Artificial
  Synthetic
gtcagaagggccctaagcccgaacgcgggattgaaac

SEQ ID NO: 268 - DNA, 37 bp, Artificial
  Synthetic
gtttcaattaacataaatccctatcagggattgaaac

SEQ ID NO: 269 - DNA, 37 bp, Artificial
  Synthetic
gtttcaatcccattactaggattcattaaaaagaaac

SEQ ID NO: 270 - DNA, 37 bp, Artificial
  Synthetic
gtttaaattattattaatacctatcagggattgaaac

SEQ ID NO: 271 - DNA, 37 bp, Artificial
  Synthetic
gtcgcgacgcggtagaaacccagaacgggattgaaac

SEQ ID NO: 272 - DNA, 37 bp, Artificial
  Synthetic
gtttcaatccctaatagggagtaagtgaaattgcaat

SEQ ID NO: 273 - DNA, 37 bp, Artificial
  Synthetic
gttcaaatttagacttatctccataggagattgaaac

SEQ ID NO: 274 - DNA, 37 bp, Artificial
  Synthetic
gtttcaatctcaaccgagatgccaggcccctggcgac

SEQ ID NO: 275 - DNA, 37 bp, Artificial
  Synthetic
ctttccaaccactaaatcccgattacgggactgaaac

SEQ ID NO: 276 - DNA, 37 bp, Artificial
  Synthetic
ctttcagtccccttgtcatcggggaaagtagtgcaac

SEQ ID NO: 277 - DNA, 37 bp, Artificial
  Synthetic
gtttcagtcccgcaagcaggattattttaattgaaag

SEQ ID NO: 278 - DNA, 30 bp, Artificial
  Synthetic
gttaaaatagaaccttaataggattgaaag

SEQ ID NO: 279 - DNA, 28 bp, Artificial
  Synthetic
ctcataaccccttatagggattcgtaac

SEQ ID NO: 280 - DNA, 28 bp, Artificial
  Synthetic
ggaccatccccgcgggcgcggggccgac

SEQ ID NO: 281 - DNA, 29 bp, Artificial
  Synthetic
gtgttccccgtacccacggggctgaaccg

SEQ ID NO: 282 - DNA, 29 bp, Artificial
  Synthetic
gtaatccccacgcacgtggggatggaccg

SEQ ID NO: 283 - DNA, 28 bp, Artificial
  Synthetic
gtattccccacgtatgtgggggtgatcc

SEQ ID NO: 284 - DNA, 29 bp, Artificial
  Synthetic
attctccccgtattcgcggggatgatccc

SEQ ID NO: 285 - DNA, 29 bp, Artificial
  Synthetic
cggttcatccccacgggtgtggggaacgc

SEQ ID NO: 286 - DNA, 29 bp, Artificial
  Synthetic
ggtttccccgcccgcgcggggatggtccc

SEQ ID NO: 287 - DNA, 29 bp, Artificial
  Synthetic
gtcctccccgcccacgcgggggtgttccg

SEQ ID NO: 288 - DNA, 29 bp, Artificial
  Synthetic
gtgctctccgcgcgagcggaggtggtccg

SEQ ID NO: 289 - DNA, 29 bp, Artificial
  Synthetic
cttttctccgcgtatgcggaggtagttcc

SEQ ID NO: 290 - DNA, 28 bp, Artificial
  Synthetic
ggctcatccccgcgggtgcggggcggac

SEQ ID NO: 291 - DNA, 28 bp, Artificial
  Synthetic
ggatcatccccgcatacgcggggagcac

SEQ ID NO: 292 - DNA, 29 bp, Artificial
  Synthetic
gtccgctccgcgcaagcggaggtgagccg

SEQ ID NO: 293 - DNA, 29 bp, Artificial
  Synthetic
gggaccatccccgcgggcgcggggagcag

SEQ ID NO: 294 - DNA, 29 bp, Artificial
  Synthetic
cggttcatccccgcgcctgcggggaacgg

SEQ ID NO: 295 - DNA, 29 bp, Artificial
  Synthetic
cggtttatccccgtgcatacggggaacac

SEQ ID NO: 296 - DNA, 29 bp, Artificial
  Synthetic
gtgttccccacacacgtggggatgaaccg

SEQ ID NO: 297 - DNA, 29 bp, Artificial
  Synthetic
gggcctatccccgctcgcgcgggggaacc

SEQ ID NO: 298 - DNA, 28 bp, Artificial
  Synthetic
ggtcgatccgcgcgtacgcggggggcac

SEQ ID NO: 299 - DNA, 28 bp, Artificial
  Synthetic
ggatcacccccgcatacgcggggaacac

SEQ ID NO: 300 - DNA, 28 bp, Artificial
  Synthetic
ggttcctccgcgtgcgcggagatagacc

SEQ ID NO: 301 - DNA, 29 bp, Artificial
  Synthetic
ctattccccacgcatgtgggggtgaaccg

SEQ ID NO: 302 - DNA, 29 bp, Artificial
  Synthetic
ggaaatacctccgcgtgtgcggagaagag

SEQ ID NO: 303 - DNA, 29 bp, Artificial
  Synthetic
cggttcatccccacgcgcgtggggaacac

SEQ ID NO: 304 - DNA, 29 bp, Artificial
  Synthetic
cggttcacccccacgtgcgtggggacaac

SEQ ID NO: 305 - DNA, 28 bp, Artificial
  Synthetic
ggatcatccccgctcgcgcggggtttac

SEQ ID NO: 306 - DNA, 29 bp, Artificial
  Synthetic
cggttcatccccacgcgtgtggggaatgc

SEQ ID NO: 307 - DNA, 28 bp, Artificial
  Synthetic
ggaacacccccgcacgcgcggggacgac

SEQ ID NO: 308 - DNA, 29 bp, Artificial
  Synthetic
gttctccccacgcgcgtggggatggtccg

SEQ ID NO: 309 - DNA, 28 bp, Artificial
  Synthetic
cttttccccgcatacgcgggggtgatcc

SEQ ID NO: 310 - DNA, 28 bp, Artificial
  Synthetic
ggatcacccccgcatacgcgggacaaat

SEQ ID NO: 311 - DNA, 29 bp, Artificial
  Synthetic
cggttcatccccgcgcatgcggggaacac

SEQ ID NO: 312 - DNA, 29 bp, Artificial
  Synthetic
gtgctctccgcgcgagcggaggtgagccg

SEQ ID NO: 313 - DNA, 29 bp, Artificial
  Synthetic
ggaattacccccgctcgcgcggggaaaag

SEQ ID NO: 314 - DNA, 28 bp, Artificial
  Synthetic
tttctaaactgcctatacggcattgaag

SEQ ID NO: 315 - DNA, 28 bp, Artificial
  Synthetic
gttcatggcggcatacgccatttagaaa

SEQ ID NO: 316 - DNA, 28 bp, Artificial
  Synthetic
gtgaactgccgagtaggtagctgataat

SEQ ID NO: 317 - DNA, 28 bp, Artificial
  Synthetic
tttctgagctgcctactcggcagtgaac

SEQ ID NO: 318 - DNA, 28 bp, Artificial
  Synthetic
gttatccgccgcacaggtggcttagaaa

SEQ ID NO: 319 - DNA, 28 bp, Artificial
  Synthetic
tttctaagccatctacacgatggtgaac

SEQ ID NO: 320 - DNA, 28 bp, Artificial
  Synthetic
cttctcaatcgcctacgcggcgataaac

SEQ ID NO: 321 - DNA, 28 bp, Artificial
  Synthetic
tttctaagctgcctgtgcggcagtgaac

SEQ ID NO: 322 - DNA, 28 bp, Artificial
  Synthetic
gttaaccgccacgcaggcggcttagaaa

SEQ ID NO: 323 - DNA, 28 bp, Artificial
  Synthetic
gttcgtcatcgcatagatgatttagaaa

SEQ ID NO: 324 - DNA, 28 bp, Artificial
  Synthetic
gtttgccgccgtgcaggcggcttagaaa

SEQ ID NO: 325 - DNA, 28 bp, Artificial
  Synthetic
gctccccgccggataggcggctgagaga

SEQ ID NO: 326 - DNA, 28 bp, Artificial
  Synthetic
attctcagctgcctatgcggcaggtcac

SEQ ID NO: 327 - DNA, 28 bp, Artificial
  Synthetic
gctggccgccgcataggcggctgagaag

SEQ ID NO: 328 - DNA, 28 bp, Artificial
  Synthetic
cttctaagccgcccacgtggcggcgaac

SEQ ID NO: 329 - DNA, 24 bp, Artificial
  Synthetic
tcagctgcctattcggcggttcac

SEQ ID NO: 330 - DNA, 28 bp, Artificial
  Synthetic
cctcgctgccgcacaggcagctcagaaa

SEQ ID NO: 331 - DNA, 28 bp, Artificial
  Synthetic
gttatccgccgtgtaggcggtttagaag

SEQ ID NO: 332 - DNA, 28 bp, Artificial
  Synthetic
gtgaaccgccgtgtaggcggcttagaaa

SEQ ID NO: 333 - DNA, 28 bp, Artificial
  Synthetic
cttcaccaccgcacaggtggcttagaaa

SEQ ID NO: 334 - DNA, 28 bp, Artificial
  Synthetic
tttctaaaccatctatgcgatggcgaac

SEQ ID NO: 335 - DNA, 28 bp, Artificial
  Synthetic
gtgaactgccggataggcagctcagaaa

SEQ ID NO: 336 - DNA, 36 bp, Artificial
  Synthetic
gcttcaatgaggccgaggcgtgaagccccggaagac

SEQ ID NO: 337 - DNA, 36 bp, Artificial
  Synthetic
attgttccggtcttccaggccggacttcattgagac

SEQ ID NO: 338 - DNA, 37 bp, Artificial
  Synthetic
gcttcaatggggccggagcgaatatgctccggaaaat

SEQ ID NO: 339 - DNA, 36 bp, Artificial
  Synthetic
gtctcttccgcttatgcggaagactttcattgaggc

SEQ ID NO: 340 - DNA, 36 bp, Artificial
  Synthetic
cttgcatacgtcaaaacgtatgcacttcattgagga

SEQ ID NO: 341 - DNA, 36 bp, Artificial
  Synthetic
gcctcaacgaagggctcccctggaaaggggagcgat

SEQ ID NO: 342 - DNA, 36 bp, Artificial
  Synthetic
ccctcaatgaaaggccgtcccataaaggacggcgac

SEQ ID NO: 343 - DNA, 36 bp, Artificial
  Synthetic
aattccgcgaccgcgcggtcgcggccccattgaagc

SEQ ID NO: 344 - DNA, 36 bp, Artificial
  Synthetic
atctccgaagtctcggcttcggagcttcattgaggg

SEQ ID NO: 345 - DNA, 36 bp, Artificial
  Synthetic
gccgcaacggagcccgctcattacgagcgggatgac

SEQ ID NO: 346 - DNA, 36 bp, Artificial
  Synthetic
gccgcaatggagcccgcccctgaaggccgggagaac

SEQ ID NO: 347 - DNA, 36 bp, Artificial
  Synthetic
gagcctcaggcccaaaagcctgagcccaattgaagc

SEQ ID NO: 348 - DNA, 30 bp, Artificial
  Synthetic
gcctcaatgaaagtcccctcccgaaggagg

SEQ ID NO: 349 - DNA, 36 bp, Artificial
  Synthetic
gcctcggcttaactgccgaggcaacttcgttgaggc

SEQ ID NO: 350 - DNA, 36 bp, Artificial
  Synthetic
gcttcaatggagccatgtcttttcagacatggtaag

SEQ ID NO: 351 - DNA, 36 bp, Artificial
  Synthetic
gcttcaatgaggccccggcaaaatgcccggggaaac

SEQ ID NO: 352 - DNA, 36 bp, Artificial
  Synthetic
gcctcaatggtgtccggccaaaatgaccggattaat

SEQ ID NO: 353 - DNA, 36 bp, Artificial
  Synthetic
ctttgatgcagccacggcggttatcgccatggacac

SEQ ID NO: 354 - DNA, 37 bp, Artificial
  Synthetic
gtgtcgccggggcttcggccccggccttcgttgagcg

SEQ ID NO: 355 - DNA, 36 bp, Artificial
  Synthetic
gcctcaatgaagggcagcccccgaaagggctgcaac

SEQ ID NO: 356 - DNA, 34 bp, Artificial
  Synthetic
gccgcaatggtccccgctgatcagcggggaggac

SEQ ID NO: 357 - DNA, 36 bp, Artificial
  Synthetic
ctctccgtcgtcctcggacgacggcttcgttgaagc

SEQ ID NO: 358 - DNA, 36 bp, Artificial
  Synthetic
ctttccgctgatctcgatcagcggccccgttgaagc

SEQ ID NO: 359 - DNA, 36 bp, Artificial
  Synthetic
gcttcaatggagccgcagtctcacagccgcggaaac

SEQ ID NO: 360 - DNA, 28 bp, Artificial
  Synthetic
cctctacccccgcatatgcggggcaaac

SEQ ID NO: 361 - DNA, 28 bp, Artificial
  Synthetic
cctctacccccgcgtgcgcggggctgac

SEQ ID NO: 362 - DNA, 36 bp, Artificial
  Synthetic
gtcgcaatggagcctgacctttgaggtcaggaaaag

SEQ ID NO: 363 - DNA, 36 bp, Artificial
  Synthetic
gcttcaatgaggcccgggcgtttcggcccggaaggc

SEQ ID NO: 364 - DNA, 36 bp, Artificial
  Synthetic
gcttcaatggggccgccgacgatctcggcggaaaac

SEQ ID NO: 365 - DNA, 36 bp, Artificial
  Synthetic
gttttccggggcataatgccccggcctcattgaagc

SEQ ID NO: 366 - DNA, 36 bp, Artificial
  Synthetic
gcttcaatggggccgcggccagatggccgcggaaac

SEQ ID NO: 367 - DNA, 36 bp, Artificial
  Synthetic
gcctcaatggagcccggtcatgaagaccgggaatac

SEQ ID NO: 368 - DNA, 35 bp, Artificial
  Synthetic
cctcaatgaaagtccctacaaaaaggagaggaaat

SEQ ID NO: 369 - DNA, 35 bp, Artificial
  Synthetic
cttcaacggggccgcgctcattcgagcgcggatgg

SEQ ID NO: 370 - DNA, 36 bp, Artificial
  Synthetic
gtttccacggctgaaaagccgtggccccattgaagc

SEQ ID NO: 371 - DNA, 36 bp, Artificial
  Synthetic
gcctcaatgaagtgcagttcccgaaggaactgcaac

SEQ ID NO: 372 - DNA, 37 bp, Artificial
  Synthetic
gcttcaatggggcccggctttgtgagccgggagtggg

SEQ ID NO: 373 - DNA, 36 bp, Artificial
  Synthetic
gcttcaacggagcccaggcccgtagacctgggtgac

SEQ ID NO: 374 - DNA, 36 bp, Artificial
  Synthetic
gcttcaatgaggccgcggcggatggccggggaaaac

SEQ ID NO: 375 - DNA, 37 bp, Artificial
  Synthetic
gttgcatccaagcttcacagcttggctacgttgcagg

SEQ ID NO: 376 - DNA, 37 bp, Artificial
  Synthetic
gttttccacggctaaccagccgtggccccattgaagc

SEQ ID NO: 377 - DNA, 36 bp, Artificial
  Synthetic
gcttcaatggggccgcgggtgtgaacccgcggatag

SEQ ID NO: 378 - DNA, 36 bp, Artificial
  Synthetic
gcttcaatgaggccgcagctcttcgctgcggatcac

SEQ ID NO: 379 - DNA, 36 bp, Artificial
  Synthetic
ctttccgcgctcttcggagcgcggccccgttgaagc

SEQ ID NO: 380 - DNA, 35 bp, Artificial
  Synthetic
gtctcatccctcccgcgggcgggcttcgttgaagg

SEQ ID NO: 381 - DNA, 37 bp, Artificial
  Synthetic
gcctcaacgaagggtccgcgaccgaagtcgcggacgg

SEQ ID NO: 382 - DNA, 36 bp, Artificial
  Synthetic
attcctgagctattaagctcaggacttcattgagga

SEQ ID NO: 383 - DNA, 36 bp, Artificial
  Synthetic
gttttccgcagcgttacgctgcggccccattgaagc

SEQ ID NO: 384 - DNA, 36 bp, Artificial
  Synthetic
gtctccgcgcgcgaaagcgagcggcttcgttgaagc

SEQ ID NO: 385 - DNA, 36 bp, Artificial
  Synthetic
gtcgccccgctcttcgcgaccgggctccattgcggc

SEQ ID NO: 386 - DNA, 36 bp, Artificial
  Synthetic
gccgcaatggagcccagcttttggagctgggaaaag

SEQ ID NO: 387 - DNA, 36 bp, Artificial
  Synthetic
ccctcaatgaaagtcacctgttctcacaggtgagac

SEQ ID NO: 388 - DNA, 36 bp, Artificial
  Synthetic
acctcaatgaaaggctgcgaccgaagccgcagcgac

SEQ ID NO: 389 - DNA, 36 bp, Artificial
  Synthetic
gtcgccgcccctcagcgggcggcccttcatcgaggc

SEQ ID NO: 390 - DNA, 36 bp, Artificial
  Synthetic
gcttcaattgggcctcgacctcgcggtcgaggaaac

SEQ ID NO: 391 - DNA, 36 bp, Artificial
  Synthetic
attttagtacctaaacaaattacgtgactgtaaaac

SEQ ID NO: 392 - DNA, 36 bp, Artificial
  Synthetic
gttttagtactctctaaattttcgtaacaataaaac

SEQ ID NO: 393 - DNA, 36 bp, Artificial
  Synthetic
gtttcagtaccattcaatataacactactgtaaaac

SEQ ID NO: 394 - DNA, 35 bp, Artificial
  Synthetic
gtttgagagtgatgtaatcttatatagtgactaat

SEQ ID NO: 395 - DNA, 36 bp, Artificial
  Synthetic
gttttaccactatctggaattacatcactctcaaac

SEQ ID NO: 396 - DNA, 36 bp, Artificial
  Synthetic
gttttagaaggatgttaaatcaataaggttaaaccc

SEQ ID NO: 397 - DNA, 36 bp, Artificial
  Synthetic
gttgtgttaccctcgtaatttttgctatctaacaac

SEQ ID NO: 398 - DNA, 36 bp, Artificial
  Synthetic
gttttagagctgtgctgtttcgaatggttccaaaac

SEQ ID NO: 399 - DNA, 36 bp, Artificial
  Synthetic
gtttgagaatggtgtagtttcatatggtagtcaaac

SEQ ID NO: 400 - DNA, 36 bp, Artificial
  Synthetic
gttttattaccatcaaaatttacacaactctcaaac

SEQ ID NO: 401 - DNA, 36 bp, Artificial
  Synthetic
gttttggtaccctctcagtttttggtatagaaaaac

SEQ ID NO: 402 - DNA, 36 bp, Artificial
  Synthetic
gtttgagagtagtgttatcttataaaactctctcac

SEQ ID NO: 403 - DNA, 35 bp, Artificial
  Synthetic
gtttgagagttgtgttgttttagataggtctaaac

SEQ ID NO: 404 - DNA, 36 bp, Artificial
  Synthetic
gctcttgaactgattgattcgacatctacctgagac

SEQ ID NO: 405 - DNA, 36 bp, Artificial
  Synthetic
gtttgagagctttgttaattcagaaagatgtaaaac

SEQ ID NO: 406 - DNA, 36 bp, Artificial
  Synthetic
gtttgagagtagtgtcatatccaatccatctcaaac

SEQ ID NO: 407 - DNA, 36 bp, Artificial
  Synthetic
gtttgagacccctatgaaattacactgctctcaaac

SEQ ID NO: 408 - DNA, 32 bp, Artificial
  Synthetic
cttgtgtattcaaaataacaatactctcaaac

SEQ ID NO: 409 - DNA, 36 bp, Artificial
  Synthetic
gtttgagagtgttgtcaaataggagtcagaccaatc

SEQ ID NO: 410 - DNA, 36 bp, Artificial
  Synthetic
gttttagagttgtgttattttgaacggttacaaaac

SEQ ID NO: 411 - DNA, 34 bp, Artificial
  Synthetic
gttttagtactctgtaatttttcgctatgataaa

SEQ ID NO: 412 - DNA, 36 bp, Artificial
  Synthetic
ggatgaaaccccattaatctgacatacatctgaagc

SEQ ID NO: 413 - DNA, 36 bp, Artificial
  Synthetic
gttgtacagttacttaaatcttgagagtacaaaaac

SEQ ID NO: 414 - DNA, 36 bp, Artificial
  Synthetic
gttttgtagaattcaaaatttacatatctctcaaac

SEQ ID NO: 415 - DNA, 36 bp, Artificial
  Synthetic
gtttcagatgcctgtcagatcaatgactttgaccac

SEQ ID NO: 416 - DNA, 36 bp, Artificial
  Synthetic
gtttgattcccctacgaatttgcactgctctcaaac

SEQ ID NO: 417 - DNA, 36 bp, Artificial
  Synthetic
gtttgagaatgatgtaaaaatgtatggtactcaaac

SEQ ID NO: 418 - DNA, 36 bp, Artificial
  Synthetic
gttttactactctctaaaattacattggtctaaaac

SEQ ID NO: 419 - DNA, 36 bp, Artificial
  Synthetic
gtttcttacccatcataatttacaaggttctcaaac

SEQ ID NO: 420 - DNA, 36 bp, Artificial
  Synthetic
gttttacaactatctgaaattacatcactctcaaac

SEQ ID NO: 421 - DNA, 36 bp, Artificial
  Synthetic
gtttgagagttatgtaatttcatataggtctaaaac

SEQ ID NO: 422 - DNA, 36 bp, Artificial
  Synthetic
gtcttattctagcaaaaaatcatatggtaacaaaac

SEQ ID NO: 423 - DNA, 36 bp, Artificial
  Synthetic
ggttcttaactaattagaatgacgtacttctaaaac

SEQ ID NO: 424 - DNA, 36 bp, Artificial
  Synthetic
gtttgagagtaatgttattttagatagatataaaac

SEQ ID NO: 425 - DNA, 36 bp, Artificial
  Synthetic
gtttgattaccagtcagaatgtcactgctccaaaac

SEQ ID NO: 426 - DNA, 36 bp, Artificial
  Synthetic
gtttcagaaggatgttaaatcaataaggttaagatc

SEQ ID NO: 427 - DNA, 36 bp, Artificial
  Synthetic
gttttagtactctgtaattttaggtataagtgaaac

SEQ ID NO: 428 - DNA, 36 bp, Artificial
  Synthetic
gttttagactacataagaatgataccacaccaaaac

SEQ ID NO: 429 - DNA, 36 bp, Artificial
  Synthetic
gtttgagaactatgtaaattatgctggtagcaaaac

SEQ ID NO: 430 - DNA, 36 bp, Artificial
  Synthetic
gttttactaccttactgatttacaaggttctcaaac

SEQ ID NO: 431 - DNA, 36 bp, Artificial
  Synthetic
gttttagaaggttgttaaatcagtaagttgaaaaac

SEQ ID NO: 432 - DNA, 36 bp, Artificial
  Synthetic
gtcttggatgagtgtcagatcagtagttccgagtac

SEQ ID NO: 433 - DNA, 36 bp, Artificial
  Synthetic
gttcaactataccgaaaaacgggagagtaccgaaac

SEQ ID NO: 434 - DNA, 36 bp, Artificial
  Synthetic
gttaaagcccagtcttaaattacacaactctcaaac

SEQ ID NO: 435 - DNA, 36 bp, Artificial
  Synthetic
gttttagaagtatgttaaatcaatgaggtttagaac

SEQ ID NO: 436 - DNA, 36 bp, Artificial
  Synthetic
gttttactgtttctaaattctttaaggtacaaaaac

SEQ ID NO: 437 - DNA, 36 bp, Artificial
  Synthetic
gttattgcttacacaagtattgtacagtgctaaaac

SEQ ID NO: 438 - DNA, 36 bp, Artificial
  Synthetic
gtttttgtgctgtacaatttcttattagagtaaaac

SEQ ID NO: 439 - DNA, 36 bp, Artificial
  Synthetic
gttttagaagagtatcaaatcaatgagtagttcaac

SEQ ID NO: 440 - DNA, 36 bp, Artificial
  Synthetic
gttctgctaccatcgaaatttttgctaggctacaac

SEQ ID NO: 441 - DNA, 36 bp, Artificial
  Synthetic
gttttagaggtttacagaattacactactctcaaac

SEQ ID NO: 442 - DNA, 36 bp, Artificial
  Synthetic
gtttgagagtattgttaaatttgaattatcttcatc

SEQ ID NO: 443 - DNA, 36 bp, Artificial
  Synthetic
gttctaaacattattgatttgaagtacatctaaaac

SEQ ID NO: 444 - DNA, 36 bp, Artificial
  Synthetic
gttcataacatcattgatatgacatccagctgaaac

SEQ ID NO: 445 - DNA, 36 bp, Artificial
  Synthetic
gttttagaattgtgttattttagatagtaataaaac

SEQ ID NO: 446 - DNA, 36 bp, Artificial
  Synthetic
gatacagattcttggtaaatttgaataatgtgaatg

SEQ ID NO: 447 - DNA, 36 bp, Artificial
  Synthetic
gttcttaaccttattgatttgtcactcaactaaaac

SEQ ID NO: 448 - DNA, 36 bp, Artificial
  Synthetic
gttttacgataacttaattctttcaggtactaaaat

SEQ ID NO: 449 - DNA, 36 bp, Artificial
  Synthetic
gttttagaactatgttattttgaatggtaacaaaac

SEQ ID NO: 450 - DNA, 36 bp, Artificial
  Synthetic
gtttaaccaccatagtgatttacatcattctcaaac

SEQ ID NO: 451 - DNA, 36 bp, Artificial
  Synthetic
gttcttaaccctattgatttaccaagattctaaagc

SEQ ID NO: 452 - DNA, 36 bp, Artificial
  Synthetic
gttttagaaggttgtctattcaataaggtttaaccc

SEQ ID NO: 453 - DNA, 36 bp, Artificial
  Synthetic
gtttaaaagtaattaaagatagcactactcccaaac

SEQ ID NO: 454 - DNA, 36 bp, Artificial
  Synthetic
gtttgagagccttgttattttgtaattctcttaaac

SEQ ID NO: 455 - DNA, 36 bp, Artificial
  Synthetic
attttggtactctctcatcttttggtataaggaaac

SEQ ID NO: 456 - DNA, 36 bp, Artificial
  Synthetic
gttttagcactatgattatttagaaagaagtaaaac

SEQ ID NO: 457 - DNA, 36 bp, Artificial
  Synthetic
gttttactaccacttgggattacactactctcaaac

SEQ ID NO: 458 - DNA, 36 bp, Artificial
  Synthetic
gatctaaacctcattgatctaacaatcatctaaaac

SEQ ID NO: 459 - DNA, 36 bp, Artificial
  Synthetic
gttctactactacataattttacagggtaccataac

SEQ ID NO: 460 - DNA, 36 bp, Artificial
  Synthetic
gtttgagaaccttgtaattttgtaagggtgtaaaac

SEQ ID NO: 461 - DNA, 36 bp, Artificial
  Synthetic
gctcttaactatattgattcgacactcttctaaaac

SEQ ID NO: 462 - DNA, 36 bp, Artificial
  Synthetic
gtttgagaaccttgtaaattacgatatctgtcaagc

SEQ ID NO: 463 - DNA, 36 bp, Artificial
  Synthetic
gattagtcactatataagattacatcactctcaaac

SEQ ID NO: 464 - DNA, 36 bp, Artificial
  Synthetic
gttctaaacctgtttgatatgactactattcaagac

SEQ ID NO: 465 - DNA, 36 bp, Artificial
  Synthetic
gttttagatgagtggtaaatctagaaggtcaaaagc

SEQ ID NO: 466 - DNA, 36 bp, Artificial
  Synthetic
gttttagaatactcaaaatttacagttaactcaaac

SEQ ID NO: 467 - DNA, 36 bp, Artificial
  Synthetic
gtagtagaaccatctggaatgacatagttccaaaac

SEQ ID NO: 468 - DNA, 36 bp, Artificial
  Synthetic
gtttgaaatccttccgtattaacaagactctcaaac

SEQ ID NO: 469 - DNA, 36 bp, Artificial
  Synthetic
gttttactaccttatgaaattacactactctcaaac

SEQ ID NO: 470 - DNA, 36 bp, Artificial
  Synthetic
gtattctaatatctatattcaacatacctagcaaac

SEQ ID NO: 471 - DNA, 36 bp, Artificial
  Synthetic
gttttcgagctatataagacaacactgctctcaaac

SEQ ID NO: 472 - DNA, 36 bp, Artificial
  Synthetic
gcttgagagtattgtaaaaccagattcctctctaac

SEQ ID NO: 473 - DNA, 36 bp, Artificial
  Synthetic
gtttaagtactatatgaatttacactactctcaaac

SEQ ID NO: 474 - DNA, 36 bp, Artificial
  Synthetic
gttttatcacattaagaatttacaaagtactaaaac

SEQ ID NO: 475 - DNA, 35 bp, Artificial
  Synthetic
gtttgagaacattcaattcaacatgactctaaaac

SEQ ID NO: 476 - DNA, 36 bp, Artificial
  Synthetic
gttttagagctatgctgttttgaatggtcccaaaac

SEQ ID NO: 477 - DNA, 36 bp, Artificial
  Synthetic
gcttcaatactacattcttctaacaactactgaaac

SEQ ID NO: 478 - DNA, 37 bp, Artificial
  Synthetic
gcgacactttacaacaatatcgcttagctaatgaaac

SEQ ID NO: 479 - DNA, 37 bp, Artificial
  Synthetic
gtttcaattgtacaaacttacatccttcaactgaaac

SEQ ID NO: 480 - DNA, 37 bp, Artificial
  Synthetic
ccaataatccctcatctaaaaatccaaccactgaaac

SEQ ID NO: 481 - DNA, 37 bp, Artificial
  Synthetic
gcaacactttatagcaaatccgcttagcctgtgaaac

SEQ ID NO: 482 - DNA, 37 bp, Artificial
  Synthetic
gttgcaaattataccacaaaccaactcggtttgaaac

SEQ ID NO: 483 - DNA, 37 bp, Artificial
  Synthetic
gtttcagtgattgagtaattggcaaaggcagtgtagg

SEQ ID NO: 484 - DNA, 37 bp, Artificial
  Synthetic
gcagagatcataacgctacgagctatagcactgaaac

SEQ ID NO: 485 - DNA, 37 bp, Artificial
  Synthetic
gtttcagttgctgaattatttggtaaactactgttag

SEQ ID NO: 486 - DNA, 37 bp, Artificial
  Synthetic
ctaacaaattataactagacacaacttagtttgaaac

SEQ ID NO: 487 - DNA, 36 bp, Artificial
  Synthetic
gtttcagttgttgttgaaagaatgtacgattgaagc

SEQ ID NO: 488 - DNA, 37 bp, Artificial
  Synthetic
ccaacacatcataacctagtaagccagcaactgaaac

SEQ ID NO: 489 - DNA, 47 bp, Artificial
  Synthetic
gttgtatttaccaatgcaaagatactaattttaaagctaatcacaac

SEQ ID NO: 490 - DNA, 47 bp, Artificial
  Synthetic
gttgtgatttgcttgaaaacgactacctttgtggtatctaaaacaac

SEQ ID NO: 491 - DNA, 47 bp, Artificial
  Synthetic
gttgtgatttgctttcaaattagtatctttgaaccattgggaacagc

SEQ ID NO: 492 - DNA, 47 bp, Artificial
  Synthetic
cttgtgattactcacataaaaataagaatttgaaagcaattcacaac

SEQ ID NO: 493 - DNA, 44 bp, Artificial
  Synthetic
gttgtggtttgctttcaaattgaacattgatgatattaaccaac

SEQ ID NO: 494 - DNA, 46 bp, Artificial
  Synthetic
gctgggaattataagtaaaaatacaattttgaaagcaattcacaac

SEQ ID NO: 495 - DNA, 46 bp, Artificial
  Synthetic
actgggaatagctttcagatttagtatttttgttgcaacgcacagc

SEQ ID NO: 496 - DNA, 32 bp, Artificial
  Synthetic
caaaactacaaaatttgaaagcaattcacaac

SEQ ID NO: 497 - DNA, 47 bp, Artificial
  Synthetic
gttgtgaattgctttcaaattttagtatctttgtgatagatcacaac

SEQ ID NO: 498 - DNA, 36 bp, Artificial
  Synthetic
gttttagagctgtgttgtttcgaatggttccaaaac

SEQ ID NO: 499 - DNA, 36 bp, Artificial
  Synthetic
attttaccataaagaaatttaaaaagggactaaaac

SEQ ID NO: 500 - DNA, 36 bp, Artificial
  Synthetic
gtttttgtactctcaagatttaagtaactgtacaac

SEQ ID NO: 501 - DNA, 36 bp, Artificial
  Synthetic
gttttatgatgactcaattctttcaggtactaaaac

SEQ ID NO: 502 - DNA, 37 bp, Artificial
  Synthetic
gttgtagctccctctctcaccccggatagctacaatt

SEQ ID NO: 503 - DNA, 36 bp, Artificial
  Synthetic
gttgtagctccctttctcatttcgcagtgctacaat

SEQ ID NO: 504 - DNA, 47 bp, Artificial
  Synthetic
gctgtgactaatcacgaagataataatttttgaaagcaattcacaac

SEQ ID NO: 505 - DNA, 36 bp, Artificial
  Synthetic
gttgccgctggaccgcgatttctgaaatgctatgct

SEQ ID NO: 506 - DNA, 36 bp, Artificial
  Synthetic
attttagtaactggaagatttgagttagtgtaaaac

SEQ ID NO: 507 - DNA, 36 bp, Artificial
  Synthetic
gttttagtacctggagaaaataagttatcgtaaaac

SEQ ID NO: 508 - DNA, 36 bp, Artificial
  Synthetic
agcttatcagttcagaaatcgcggtccagccggaac

SEQ ID NO: 509 - DNA, 36 bp, Artificial
  Synthetic
gatctattttagctgaaaactgaaggaatcaatagc

SEQ ID NO: 510 - DNA, 36 bp, Artificial
  Synthetic
aatatcgtgttttctaatctttaatcaaaccacaac

SEQ ID NO: 511 - DNA, 47 bp, Artificial
  Synthetic
gttgtaaattgctttcaattttccgtaactttgtgattagttacaac

SEQ ID NO: 512 - DNA, 36 bp, Artificial
  Synthetic
atcatatcataccaagatttaaagaggaattatggc

SEQ ID NO: 513 - DNA, 36 bp, Artificial
  Synthetic
attgtagccgatggggaaccggggtccagccgcaac

SEQ ID NO: 514 - DNA, 36 bp, Artificial
  Synthetic
gttgtaacagattagaaaatgcggtcaagccgcaac

SEQ ID NO: 515 - DNA, 36 bp, Artificial
  Synthetic
agtgtatccgagcgacggttcccgacgaaccacagc

SEQ ID NO: 516 - DNA, 36 bp, Artificial
  Synthetic
gctggggttcagttctcaaaaaccctgatagacttc

SEQ ID NO: 517 - DNA, 36 bp, Artificial
  Synthetic
gccgtggcttccctaccgatttccccgtggtaggct

SEQ ID NO: 518 - DNA, 46 bp, Artificial
  Synthetic
gttgtgaattgctttcagaactttaattttagtgatgattcacagt

SEQ ID NO: 519 - DNA, 36 bp, Artificial
  Synthetic
gttccggccagtgcgcatatcccagtgatctagaat

SEQ ID NO: 520 - DNA, 36 bp, Artificial
  Synthetic
caagcttatcaagaagggtgaatgctaattcccagc

SEQ ID NO: 521 - DNA, 36 bp, Artificial
  Synthetic
agtttagctgttcagaattcggggtccagccgcaac

SEQ ID NO: 522 - DNA, 47 bp, Artificial
  Synthetic
gttgtgatttgctttcaaatttttatctttgacatattaaaaacagc

SEQ ID NO: 523 - DNA, 36 bp, Artificial
  Synthetic
gtcatagttcccctgagattatcgctgtggtataat

SEQ ID NO: 524 - DNA, 36 bp, Artificial
  Synthetic
gttttgccttgaatccaaaataaggcacagtacaat

SEQ ID NO: 525 - DNA, 36 bp, Artificial
  Synthetic
gttatagttcctagtaaattctcgatatgctataat

SEQ ID NO: 526 - DNA, 36 bp, Artificial
  Synthetic
gttccggtcagagctcaaatcccaatctgctaaact

SEQ ID NO: 527 - DNA, 36 bp, Artificial
  Synthetic
attttaccatataaacaattaataataggctaaaac

SEQ ID NO: 528 - DNA, 36 bp, Artificial
  Synthetic
aggttagcagatcgggaattgcgctctggctacaac

SEQ ID NO: 529 - DNA, 47 bp, Artificial
  Synthetic
gttgtgcagtatcacaaagatacagttaaatgtaagcaattcacaac

SEQ ID NO: 530 - DNA, 30 bp, Artificial
  Synthetic
gtttccatccctcatagtcacttctaaaac

SEQ ID NO: 531 - DNA, 37 bp, Artificial
  Synthetic
gtcacaatccttattttaatagaatattctttgcaat

SEQ ID NO: 532 - DNA, 36 bp, Artificial
  Synthetic
gtcagaaagaatgccccgattagaggggattgagac

SEQ ID NO: 533 - DNA, 32 bp, Artificial
  Synthetic
gttttcgtccccatgattggggatcttttttt

SEQ ID NO: 534 - DNA, 35 bp, Artificial
  Synthetic
gtttccgtccccttacggggattatggttcttaac

SEQ ID NO: 535 - DNA, 37 bp, Artificial
  Synthetic
gtcaaaaagtagattccactaaaacaaggattgaaac

SEQ ID NO: 536 - DNA, 33 bp, Artificial
  Synthetic
gtttcaatccttgttttgatagactgttgcaac

SEQ ID NO: 537 - DNA, 36 bp, Artificial
  Synthetic
gaaacacccatgccctgatttcaaagggattgagac

SEQ ID NO: 538 - DNA, 35 bp, Artificial
  Synthetic
gtcgtaatcccttctaaatcaggtctaattctttc

SEQ ID NO: 539 - DNA, 35 bp, Artificial
  Synthetic
gttttaatagcatcaccccgcaaggggacggaaac

SEQ ID NO: 540 - DNA, 36 bp, Artificial
  Synthetic
gttctcgtcccctccacggggttctcttatctcaat

SEQ ID NO: 541 - DNA, 36 bp, Artificial
  Synthetic
gtcttaatcccctagtgatggggctttgtttttaat

SEQ ID NO: 542 - DNA, 35 bp, Artificial
  Synthetic
gtttccgtccccttgcggggttctggtacttaaat

SEQ ID NO: 543 - DNA, 27 bp, Artificial
  Synthetic
gttgcatccccgaaatgggattaaaac

SEQ ID NO: 544 - DNA, 36 bp, Artificial
  Synthetic
gttctcgtcccctattactggggataatctaatacc

SEQ ID NO: 545 - DNA, 34 bp, Artificial
  Synthetic
atttgaaaaacactccccgtgaggggacggaaac

SEQ ID NO: 546 - DNA, 36 bp, Artificial
  Synthetic
attagaagatagagaaccccagtaggggacggaaac

SEQ ID NO: 547 - DNA, 36 bp, Artificial
  Synthetic
aatcagagaataccccgtataaaaggggacgagaac

SEQ ID NO: 548 - DNA, 35 bp, Artificial
  Synthetic
gtttatgaataagatccccgaaaggggacggaaac

SEQ ID NO: 549 - DNA, 32 bp, Artificial
  Synthetic
gttaccgtccccttgcgggggtaagttgtttc

SEQ ID NO: 550 - DNA, 36 bp, Artificial
  Synthetic
gtcttaatcccctttagatcgggtcagccttcaaac

SEQ ID NO: 551 - DNA, 35 bp, Artificial
  Synthetic
gaatgaagagacctgaactacaaagggattacgac

SEQ ID NO: 552 - DNA, 36 bp, Artificial
  Synthetic
gttctcgtctccttctagcggagataagttgttagc

SEQ ID NO: 553 - DNA, 36 bp, Artificial
  Synthetic
attgagatataagaaccccggggaggggacgaaaac

SEQ ID NO: 554 - DNA, 31 bp, Artificial
  Synthetic
aaaaaaataatcccctaatggggacggaaac

SEQ ID NO: 555 - DNA, 36 bp, Artificial
  Synthetic
attaaaaacgaaggacccctttaaggggacggaaac

SEQ ID NO: 556 - DNA, 35 bp, Artificial
  Synthetic
gttaaattaaataatcccctgatggggacggaaac

SEQ ID NO: 557 - DNA, 37 bp, Artificial
  Synthetic
gtcttaatccttgttttgctggaatatggtttctgac

SEQ ID NO: 558 - DNA, 33 bp, Artificial
  Synthetic
gtttcgatgctccatgttttgagctttttttat

SEQ ID NO: 559 - DNA, 31 bp, Artificial
  Synthetic
atattaatcgcaccatttaaggaattgaaac

SEQ ID NO: 560 - DNA, 36 bp, Artificial
  Synthetic
gtttccgtctcctctcggagttatattctctcttat

SEQ ID NO: 561 - DNA, 36 bp, Artificial
  Synthetic
gttaaaatcaagccctgaaataaaagggattgagac

SEQ ID NO: 562 - DNA, 34 bp, Artificial
  Synthetic
ttaaattaaatgatccccattaggggacggaaac

SEQ ID NO: 563 - DNA, 34 bp, Artificial
  Synthetic
gtttccgtcccctctttcggggatgttttttatt

SEQ ID NO: 564 - DNA, 33 bp, Artificial
  Synthetic
gtcgctcccttcacggggagcgtggattgaaat

SEQ ID NO: 565 - DNA, 32 bp, Artificial
  Synthetic
gttatcgtccccttgcggggataagctgcaat

SEQ ID NO: 566 - DNA, 32 bp, Artificial
  Synthetic
atttcaatccacgaggctgtataagcctcgac

SEQ ID NO: 567 - DNA, 31 bp, Artificial
  Synthetic
gaaccaacccatccccaagcggggacgaaaa

SEQ ID NO: 568 - DNA, 31 bp, Artificial
  Synthetic
gttgaatattgttcttattaaggattaacgc

SEQ ID NO: 569 - DNA, 25 bp, Artificial
  Synthetic
ttaaatacatcttatgttactgttc

SEQ ID NO: 570 - DNA, 31 bp, Artificial
  Synthetic
gttagatatcatacttattaaggattaacgc

SEQ ID NO: 571 - DNA, 30 bp, Artificial
  Synthetic
gttttcgtacagtatctgttgatatttatc

SEQ ID NO: 572 - DNA, 37 bp, Artificial
  Synthetic
gtcttaatccttgttgtaatggaatatacttaataat

SEQ ID NO: 573 - DNA, 30 bp, Artificial; identical to SEQ ID NO: 114
  Synthetic

SEQ ID NO: 574 - DNA, 30 bp, Artificial
  Synthetic
gtttttagcctacctataagggattgaaac

SEQ ID NO: 575 - DNA, 30 bp, Artificial
  Synthetic
gtttcaattcctcataggtacgttcaaaac

SEQ ID NO: 576 - DNA, 30 bp, Artificial
  Synthetic
gtttcaattctatgatagtccgattaaaag

SEQ ID NO: 577 - DNA, 30 bp, Artificial
  Synthetic
gtttaaatagaaacatactgtaatgtaaat

SEQ ID NO: 578 - DNA, 36 bp, Artificial
  Synthetic
attaagaaaataaaagccccgataggggacgaaaac

SEQ ID NO: 579 - DNA, 27 bp, Artificial
  Synthetic
gtattatccacacgaacaaggattgaa

SEQ ID NO: 580 - DNA, 37 bp, Artificial
  Synthetic
gtcttaatccttgttttagtggaagttagtctgtgag

SEQ ID NO: 581 - DNA, 29 bp, Artificial
  Synthetic
gtctccatcccacacggtgggcttagaac

SEQ ID NO: 582 - DNA, 28 bp, Artificial
  Synthetic
cttccatactaactagtacttcttaaac

SEQ ID NO: 583 - DNA, 28 bp, Artificial
  Synthetic
tttctaaactacctatgcggcagttaac

SEQ ID NO: 584 - DNA, 36 bp, Artificial
  Synthetic
gtcgcaatccccttacggggctaaatcgcttgcaac

SEQ ID NO: 585 - DNA, 27 bp, Artificial
  Synthetic
gttttcgcccccttacggggatggtac

SEQ ID NO: 586 - DNA, 25 bp, Artificial
  Synthetic
ctttcaattccttttgggatgcaac

SEQ ID NO: 587 - DNA, 37 bp, Artificial
  Synthetic
atttgaataacagaccttattaagaagggattaagac

SEQ ID NO: 588 - DNA, 36 bp, Artificial
  Synthetic
gtcgtcagacccaaaaccccgagaggggacggaaac

SEQ ID NO: 589 - DNA, 37 bp, Artificial
  Synthetic
gtcgaaagacattgccctgttccaaagggattgagac

SEQ ID NO: 590 - DNA, 37 bp, Artificial
  Synthetic
atttaaacaaatttgacccgatcaaggggattgaaac

SEQ ID NO: 591 - DNA, 36 bp, Artificial
  Synthetic
gtctgaatgattccctgatttagaagggattaagac

SEQ ID NO: 592 - DNA, 36 bp, Artificial
  Synthetic
gtttccgtctccttacggagtatcattcattcttat

SEQ ID NO: 593 - DNA, 36 bp, Artificial
  Synthetic
gtaagaatggaatgacctgcttgaggggattgagac

SEQ ID NO: 594 - DNA, 36 bp, Artificial
  Synthetic
atagaaataaaaaaatccccgtgaggggacggaaac

SEQ ID NO: 595 - DNA, 37 bp, Artificial
  Synthetic
gtttccgtcccctctcgaggtgactggggtgtcttac

SEQ ID NO: 596 - DNA, 36 bp, Artificial
  Synthetic
gtcgcaatcccttttaaatcaggtcaagtttcctac

SEQ ID NO: 597 - DNA, 37 bp, Artificial
  Synthetic
gtcttaatcccttcttaataaggtctctggtttaaat

SEQ ID NO: 598 - DNA, 36 bp, Artificial
  Synthetic
gttagaacgctgccctgaattaaaagggattgagac

SEQ ID NO: 599 - DNA, 36 bp, Artificial
  Synthetic
gtcacaatccttgttttagtggacagtttttgcaat

SEQ ID NO: 600 - DNA, 36 bp, Artificial
  Synthetic
attcggagcaagatccactaaaacaaggattgaaac

SEQ ID NO: 601 - DNA, 37 bp, Artificial
  Synthetic
gtctgaaaagacctaatccccgcaaggggactgaaac

SEQ ID NO: 602 - DNA, 33 bp, Artificial
  Synthetic
gttgcaacttacaccccacaaggggacggaaac

SEQ ID NO: 603 - DNA, 36 bp, Artificial
  Synthetic
attgagaaatgtagctccccgataggggacggaaac

SEQ ID NO: 604 - DNA, 35 bp, Artificial
  Synthetic
gtcgcaaacaaaaagccccgtaaggggattgcaac

SEQ ID NO: 605 - DNA, 36 bp, Artificial
  Synthetic
gatataaacctaattacctcgagaggggacggaaac

SEQ ID NO: 606 - DNA, 36 bp, Artificial
  Synthetic
gataattacctatctccgagttaaggagacgaaaac

SEQ ID NO: 607 - DNA, 37 bp, Artificial
  Synthetic
gtttcagtccccttccttacggggtaacggccatctc

SEQ ID NO: 608 - DNA, 35 bp, Artificial
  Synthetic
gtcgcaatcccttcgtaaatcaggtcaattctttc

SEQ ID NO: 609 - DNA, 36 bp, Artificial
  Synthetic
gttctcgtcccctattcttcggggtagttatcgatc

SEQ ID NO: 610 - DNA, 36 bp, Artificial
  Synthetic
gtctcaatcccttttagaacagggctaggttagaac

SEQ ID NO: 611 - DNA, 36 bp, Artificial
  Synthetic
gtcttaatccttattatactggaagtactctatgag

SEQ ID NO: 612 - DNA, 37 bp, Artificial
  Synthetic
gtcgcaatcccctgaaaacgggtctcgtccctgcaag

SEQ ID NO: 613 - DNA, 37 bp, Artificial
  Synthetic
gtccttacggacgctccctgactgaagggattaagac

SEQ ID NO: 614 - DNA, 37 bp, Artificial
  Synthetic
gtcgcaatcccttattcatcaggtctctgtttccaac

SEQ ID NO: 615 - DNA, 36 bp, Artificial
  Synthetic
attaaaaagaattgaactccgaaaggagacggaaac

SEQ ID NO: 616 - DNA, 36 bp, Artificial
  Synthetic
gtttccgtcccttacaggggttattcgttttttaat

SEQ ID NO: 617 - DNA, 30 bp, Artificial
  Synthetic
tacctatctacctcgagaggggccggaaac

SEQ ID NO: 618 - DNA, 33 bp, Artificial
  Synthetic
atttcaaccttatccccgcaaggggacggaaac

SEQ ID NO: 619 - DNA, 35 bp, Artificial
  Synthetic
gtttccgtcccctaatggggatcatttaatttaac

SEQ ID NO: 620 - DNA, 37 bp, Artificial
  Synthetic
gtcttaatccttgttttaatggaagatactctctgag

SEQ ID NO: 621 - DNA, 36 bp, Artificial
  Synthetic
cttcaaaaaaccaaaatccccgcgaggggattgaaa

SEQ ID NO: 622 - DNA, 36 bp, Artificial
  Synthetic
gtttccgtccccttatggggtttcttattttcaaat

SEQ ID NO: 623 - DNA, 25 bp, Artificial
  Synthetic
gtatcagaaaagaatgaattgaaag

SEQ ID NO: 624 - DNA, 31 bp, Artificial
  Synthetic
tgtttcaatccttcttaggtaagcccaaaac

SEQ ID NO: 625 - DNA, 36 bp, Artificial
  Synthetic
ccttacctattaggtcaaataggattagttggaaac

SEQ ID NO: 626 - DNA, 36 bp, Artificial
  Synthetic
gtcttaatccctttgatatcagggctcggttcggac

SEQ ID NO: 627 - DNA, 36 bp, Artificial
  Synthetic
gtctctatttattagagaaattaattgaatggaaac

SEQ ID NO: 628 - DNA, 36 bp, Artificial
  Synthetic
gtcccgctcgccgtgatgccggaaggcgttgagcac

SEQ ID NO: 629 - DNA, 35 bp, Artificial
  Synthetic
gtttccattctattagcttcctcctaagagaaaag

SEQ ID NO: 630 - DNA, 37 bp, Artificial
  Synthetic
ataagaataggtaatccagtaaaataaggattgaaac

SEQ ID NO: 631 - DNA, 36 bp, Artificial
  Synthetic
gtcttaatcccttagaaatcagggagcgtttcagac

SEQ ID NO: 632 - DNA, 36 bp, Artificial
  Synthetic
gtagcgactttcattagaaagaaaaggtgttgaaac

SEQ ID NO: 633 - DNA, 36 bp, Artificial
  Synthetic
ccccgcttccctctgggaagtggaatcaatggaaac

SEQ ID NO: 634 - DNA, 35 bp, Artificial
  Synthetic
gttcaaaccttccccgatttctaggggattaagac

SEQ ID NO: 635 - DNA, 32 bp, Artificial
  Synthetic
gttcaactacctttcatataaggaattaagac

SEQ ID NO: 636 - DNA, 36 bp, Artificial
  Synthetic
gtttcaatggggccacggcttttcagccatggatac

SEQ ID NO: 637 - DNA, 37 bp, Artificial
  Synthetic
gtctcgaaataactacctctcatggagagaggtttac

SEQ ID NO: 638 - DNA, 25 bp, Artificial
  Synthetic
gcagaatctacaaaagaattgaaac

SEQ ID NO: 639 - DNA, 35 bp, Artificial
  Synthetic
cttttaacttcttagcaagtttaattaatggaaac

SEQ ID NO: 640 - DNA, 36 bp, Artificial
  Synthetic
gtcgtaatcccttcaagtcaggtcattgattccaat

SEQ ID NO: 641 - DNA, 37 bp, Artificial
  Synthetic
gtttcaatccttgttttagtggatcttgctcacgaat

SEQ ID NO: 642 - DNA, 37 bp, Artificial
  Synthetic
gtcgagaagtagcttccactaaaacaaggattgaaac

SEQ ID NO: 643 - DNA, 35 bp, Artificial
  Synthetic
atttgagttacttccagtagaataaggattaagac

SEQ ID NO: 644 - DNA, 36 bp, Artificial
  Synthetic
gtcttaatcccttttgtatcagggcagcattttatc

SEQ ID NO: 645 - DNA, 35 bp, Artificial
  Synthetic
gtcgcaatactctgcgagtacgattatatcaaaag

SEQ ID NO: 646 - DNA, 37 bp, Artificial
  Synthetic
ctcactatacatacacagtccgtaaggctattaagac

SEQ ID NO: 647 - DNA, 35 bp, Artificial
  Synthetic
gtttccatacgtttaactttcaaagaagtttaaag

SEQ ID NO: 648 - DNA, 30 bp, Artificial
  Synthetic
gtttttagcttacctataaggaattgaaac

SEQ ID NO: 649 - DNA, 37 bp, Artificial
  Synthetic
ggccaaaaccaggccccgaaacgcaggggattaagac

SEQ ID NO: 650 - DNA, 32 bp, Artificial
  Synthetic
gtcgcactctacatgagtgcgtggattgaaat

SEQ ID NO: 651 - DNA, 32 bp, Artificial
  Synthetic
gtttcaatccacgctcccgcatagggagcgac

SEQ ID NO: 652 - DNA, 30 bp, Artificial
  Synthetic
gttgaacattaacatgagatgtatttaaat

SEQ ID NO: 653 - DNA, 26 bp, Artificial
  Synthetic
ataccttacctataaggaattgagac

SEQ ID NO: 654 - DNA, 30 bp, Artificial
  Synthetic
gtatcaatatctattaggtaaggtatctac

SEQ ID NO: 655 - DNA, 30 bp, Artificial
  Synthetic
atttcaatacatctaatgtttttatttatc

SEQ ID NO: 656 - DNA, 30 bp, Artificial
  Synthetic
gtttcaatccctcataggttcgattcaaac

SEQ ID NO: 657 - DNA, 29 bp, Artificial
  Synthetic
tattaggtcaaataggattagttggaaac

SEQ ID NO: 658 - DNA, 28 bp, Artificial
  Synthetic
tttctaagctgcctgtgcggcagtgaac

SEQ ID NO: 659 - DNA, 37 bp, Artificial
  Synthetic
gtatctatacatattcagtcagtaagactatcaagac

SEQ ID NO: 660 - DNA, 36 bp, Artificial
  Synthetic
tcccccactcgctaggggacgcgatcgattggaaac

SEQ ID NO: 661 - DNA, 36 bp, Artificial
  Synthetic
gtcttaatcccttctttatcaggtctgttattcaat

SEQ ID NO: 662 - DNA, 37 bp, Artificial
  Synthetic
gattcaataccagccctgaattcaaagggattaagac

SEQ ID NO: 663 - DNA, 37 bp, Artificial
  Synthetic
ctttcaataccctacgacgggtcgtaccagttgcaac

SEQ ID NO: 664 - DNA, 36 bp, Artificial
  Synthetic
gttggaaacatgccctgatttaaaagggattaagac

SEQ ID NO: 665 - DNA, 36 bp, Artificial
  Synthetic
gtcttaatccccgattagtggggcaagtcttccgac

SEQ ID NO: 666 - DNA, 36 bp, Artificial
  Synthetic
ctcgcattgatgacccgttctaaaggggattgagac

SEQ ID NO: 667 - DNA, 36 bp, Artificial
  Synthetic
ctcgcgccgactgggttaagtggcttagttggaaac

SEQ ID NO: 668 - DNA, 37 bp, Artificial
  Synthetic
ggcggcgccccctccccgatgagaaggggattgaaac

SEQ ID NO: 669 - DNA, 37 bp, Artificial
  Synthetic
cttgcgccgatcctttgcgatcggattagttggaaac

SEQ ID NO: 670 - DNA, 37 bp, Artificial
  Synthetic
gtcttaatcccttcttaatcaggtcgtttgtttggac

SEQ ID NO: 671 - DNA, 35 bp, Artificial
  Synthetic
atgtagatgtattccagtataataaggattaagac

SEQ ID NO: 672 - DNA, 36 bp, Artificial
  Synthetic
gtcaccactcgctggtgacaccaattgattggaaac

SEQ ID NO: 673 - DNA, 37 bp, Artificial
  Synthetic
gaagaaagacataccctgaagaagaagggattacgac

SEQ ID NO: 674 - DNA, 36 bp, Artificial
  Synthetic
gtctcaatcccctctaatcggggcattcgttctgac

SEQ ID NO: 675 - DNA, 36 bp, Artificial
  Synthetic
ctccccactcgctggggaaattaattgaatggaaac

SEQ ID NO: 676 - DNA, 36 bp, Artificial
  Synthetic
gtttccattcattacgcttcacgaattagaagcaag

SEQ ID NO: 677 - DNA, 36 bp, Artificial
  Synthetic
ctcagaaacccccgcccgctaatagggctttgtgac

SEQ ID NO: 678 - DNA, 30 bp, Artificial
  Synthetic
gtttttagcctacctataaggaattgaaac

SEQ ID NO: 679 - DNA, 37 bp, Artificial; identical to SEQ ID NO: 641
  Synthetic

SEQ ID NO: 680 - DNA, 37 bp, Artificial
  Synthetic
gtttctattcgctgagtcgcgagacgttcagcggaac

SEQ ID NO: 681 - DNA, 37 bp, Artificial
  Synthetic
gtcagaaagcaccgagcgccataaggtgcattaagac

SEQ ID NO: 682 - DNA, 35 bp, Artificial
  Synthetic
gtttaaaatgactcgccccgtaaggggacggaaac

SEQ ID NO: 683 - DNA, 36 bp, Artificial
  Synthetic
gtttagaaggacactaaccccgaaggggattgaaac

SEQ ID NO: 684 - DNA, 37 bp, Artificial
  Synthetic
ctcatagagtatcttccattaaaacaaggattaagac

SEQ ID NO: 685 - DNA, 36 bp, Artificial
  Synthetic
gttacgcttacacttaccccgcaaggggatggaaac

SEQ ID NO: 686 - DNA, 35 bp, Artificial
  Synthetic
gtttccatccccgtgaggggtaaatgatttaaaac

SEQ ID NO: 687 - DNA, 25 bp, Artificial
  Synthetic
gatgaatcccaaaagggattgaaag

SEQ ID NO: 688 - DNA, 35 bp, Artificial
  Synthetic
gttgcaacagtacttaccgtttacggtattgaaac

SEQ ID NO: 689 - DNA, 35 bp, Artificial
  Synthetic
gtattagtactgcccggtttagacgggattaagac

SEQ ID NO: 690 - DNA, 37 bp, Artificial
  Synthetic
gtttcaatccgcttcgaggttcgcggtggtttcagac

SEQ ID NO: 691 - DNA, 33 bp, Artificial
  Synthetic
gtttcaattcccgcaatgcgggtaagatacatc

SEQ ID NO: 692 - DNA, 36 bp, Artificial
  Synthetic
attgcatgcaatccataccgcttgcggtattgaaac

SEQ ID NO: 693 - DNA, 33 bp, Artificial
  Synthetic
gctacaattcgcgccccttgcggggactgtaac

SEQ ID NO: 694 - DNA, 33 bp, Artificial
  Synthetic
gtctctttctgtatggagagagtggattgaaat

SEQ ID NO: 695 - DNA, 33 bp, Artificial
  Synthetic
gatgcagcccgctcccctcacggggacggcaac

SEQ ID NO: 696 - DNA, 33 bp, Artificial
  Synthetic
atttcaatccacacagccttcgcaggctgtgac

SEQ ID NO: 697 - DNA, 37 bp, Artificial
  Synthetic
gtgtcaatgcccttactcgggctattcccatttctac

SEQ ID NO: 698 - DNA, 32 bp, Artificial
  Synthetic
atttcaatccacgaggcaacataagcctcgac

SEQ ID NO: 699 - DNA, 36 bp, Artificial
  Synthetic
gtcttaatccccttaaatcgggtcatgttgttaaat

SEQ ID NO: 700 - DNA, 30 bp, Artificial
  Synthetic
gtaagtaccttacctataaggaatggaaac

SEQ ID NO: 701 - DNA, 30 bp, Artificial
  Synthetic
gtttttaccctacctatgaggaattgaaac

SEQ ID NO: 702 - DNA, 29 bp, Artificial
  Synthetic
gtcggacattacctatgaggaattgatac

SEQ ID NO: 703 - DNA, 30 bp, Artificial
  Synthetic
gttgcgatccctccaggggtgatcagcgac

SEQ ID NO: 704 - DNA, 30 bp, Artificial
  Synthetic
gttgcgacccctcttaggggcgatgaggac

SEQ ID NO: 705 - DNA, 30 bp, Artificial
  Synthetic
atttaaatacatctcatgttaatgttcaac

SEQ ID NO: 706 - DNA, 37 bp, Artificial
  Synthetic
gtttcaatccttattttactggattctcttcttttat

SEQ ID NO: 707 - DNA, 37 bp, Artificial
  Synthetic
gtccggcagcatgtgccctcaatcaagggattaagac

SEQ ID NO: 708 - DNA, 37 bp, Artificial; identical to SEQ ID NO: 641
  Synthetic

SEQ ID NO: 709 - DNA, 36 bp, Artificial
  Synthetic
gtctgaaccaccccctcaccgcgaggtgattgaaac

SEQ ID NO: 710 - DNA, 37 bp, Artificial
  Synthetic
gttggaaaccaagtaccgcgatatagcggatggaaac

SEQ ID NO: 711 - DNA, 37 bp, Artificial
  Synthetic
gtacgagtgcatcatccattaaaacaaggattaagac

SEQ ID NO: 712 - DNA, 35 bp, Artificial
  Synthetic
gtttccgtccccttgcggggaaatggtagttaaac

SEQ ID NO: 713 - DNA, 37 bp, Artificial
  Synthetic
gtgtcaatgccctcaacgggcttctactcatttctac

SEQ ID NO: 714 - DNA, 37 bp, Artificial
  Synthetic
gtctcaatccccttctcatcggggaggcggcgccgcc

SEQ ID NO: 715 - DNA, 37 bp, Artificial
  Synthetic
gttgcaccacatacaaagctgcgtgcggcattgaaac

SEQ ID NO: 716 - DNA, 37 bp, Artificial
  Synthetic
gtttcaatccttgttttagtggaagctacttctcgac

SEQ ID NO: 717 - DNA, 37 bp, Artificial
  Synthetic
atttgattagaatttccccgatctaggggattgaaac

SEQ ID NO: 718 - DNA, 37 bp, Artificial
  Synthetic
gtttccatacgccttatggcgtgaccttgtttcggac

SEQ ID NO: 719 - DNA, 36 bp, Artificial
  Synthetic
gtcttaatcccctacaaagcggggctcctttctgac

SEQ ID NO: 720 - DNA, 37 bp, Artificial
  Synthetic
gtcttaatccttgttgtagtggacaatggtctcgtac

SEQ ID NO: 721 - DNA, 35 bp, Artificial
  Synthetic
gtcttaatcccttttaaatcagggctggttcagac

SEQ ID NO: 722 - DNA, 37 bp, Artificial
  Synthetic
gtattgaaggttatccatttataataaggtttaaaac

SEQ ID NO: 723 - DNA, 36 bp, Artificial
  Synthetic
gtaccgtgacattaagtccttctcttgtttcataac

SEQ ID NO: 724 - DNA, 36 bp, Artificial; identical to SEQ ID NO: 723
  Synthetic

SEQ ID NO: 725 - DNA, 35 bp, Artificial
  Synthetic
atagatatagacagaagcttttaatgtgatgggac

SEQ ID NO: 726 - DNA, 37 bp, Artificial
  Synthetic
gtttcaatccgcttcctctagcggcatccctcaatag

SEQ ID NO: 727 - DNA, 36 bp, Artificial
  Synthetic
cttgaagactaaaggaaggaattaatgtcacggtac

SEQ ID NO: 728 - DNA, 37 bp, Artificial
  Synthetic
gttggtgcatcagcccggaattatgatgttttggtac

SEQ ID NO: 729 - DNA, 36 bp, Artificial
  Synthetic
gtaccgtgacatcaaaaattcctgccgcctttcaac

SEQ ID NO: 730 - DNA, 36 bp, Artificial
  Synthetic
gttccgtgacattaagggtttatcggctggggcaat

SEQ ID NO: 731 - DNA, 30 bp, Artificial
  Synthetic
gttaaaatcagaccattgtgggattgaaag

SEQ ID NO: 732 - DNA, 37 bp, Artificial
  Synthetic
gttgcagaagtcgatctagtaaaacaaggattgaaac

SEQ ID NO: 733 - DNA, 38 bp, Artificial
  Synthetic
gttggaaacagagacctgatgaataagggattgcgact

SEQ ID NO: 734 - DNA, 30 bp, Artificial
  Synthetic
ctttcaatcccacaatggtctgattttaac

SEQ ID NO: 735 - DNA, 37 bp, Artificial
  Synthetic
gtttcaatccttgttttgctggaagctgtttttcaac

SEQ ID NO: 736 - DNA, 33 bp, Artificial
  Synthetic
gaaaggaactaatcctcggaaggggattgaaac

SEQ ID NO: 737 - DNA, 37 bp, Artificial
  Synthetic
ctgataaaattacccttgatgaataaaggattgcgac

SEQ ID NO: 738 - DNA, 33 bp, Artificial
  Synthetic
caatgcattgcctattgataatgctatcgatcc

SEQ ID NO: 739 - DNA, 33 bp, Artificial; identical to SEQ ID NO: 738
  Synthetic

SEQ ID NO: 740 - DNA, 33 bp, Artificial
  Synthetic
caatgcattaccctaaagtaatgctactggcac

SEQ ID NO: 741 - DNA, 33 bp, Artificial; identical to SEQ ID NO: 740
  Synthetic

SEQ ID NO: 742 - DNA, 29 bp, Artificial
  Synthetic
gtatttcccgcgtgcgcgggggtgagcgg

SEQ ID NO: 743 - DNA, 29 bp, Artificial
  Synthetic
gtatttcccgcgtgcgcgggggtgagcgg

SEQ ID NO: 744 - DNA, 29 bp, Artificial
  Synthetic
gtgcgccccgcgtgagcgggggttgaagg

SEQ ID NO: 745 - DNA, 29 bp, Artificial
  Synthetic
gtgcgccccgcgtgagcgggggttgaagg

SEQ ID NO: 746 - DNA, 37 bp, Artificial
  Synthetic
gttgtaataagctgtaaagtgttgcggctttggaaac

SEQ ID NO: 747 - DNA, 37 bp, Artificial; identical to SEQ ID NO: 746
  Synthetic

SEQ ID NO: 748 - DNA, 28 bp, Artificial
  Synthetic
cgataacccccgctcgcgcggggcagac

SEQ ID NO: 749 - DNA, 28 bp, Artificial
  Synthetic
cgataacccccgctcgcgcggggcagac

SEQ ID NO: 750 - DNA, 23 bp, Artificial
  Synthetic
cttctaaatcacctatgcggtga

SEQ ID NO: 751 - DNA, 23 bp, Artificial
  Synthetic
cttctaaatcacctatgcggtga

SEQ ID NO: 752 - DNA, 23 bp, Artificial
  Synthetic
ctttaacccccgtaggtacgggg

SEQ ID NO: 753 - DNA, 23 bp, Artificial
  Synthetic
ctttaacccccgcaggtacgggg

SEQ ID NO: 754 - DNA, 36 bp, Artificial
  Synthetic
gttccaaccacgcccccacagggacgtgctttccac

SEQ ID NO: 755 - DNA, 36 bp, Artificial; identical to SEQ ID NO: 754
  Synthetic

SEQ ID NO: 756 - DNA, 36 bp, Artificial
  Synthetic
gctcaagccaccctgtaattgggtggttactagaac

SEQ ID NO: 757 - DNA, 36 bp, Artificial; identical to SEQ ID NO: 756
  Synthetic

SEQ ID NO: 758 - DNA, 36 bp, Artificial
  Synthetic
ggtggaagaactgccccaaggggtagtggttggaac

SEQ ID NO: 759 - DNA, 36 bp, Artificial; identical to SEQ ID NO: 758
  Synthetic

SEQ ID NO: 760 - DNA, 28 bp, Artificial
  Synthetic
cggttatccgcgcccgcgtaggcgcgac

SEQ ID NO: 761 - DNA, 29 bp, Artificial
  Synthetic
gtattccccccgcatgcgggggttatcgg

SEQ ID NO: 762 - DNA, 29 bp, Artificial
  Synthetic
gtcacgcccctgcgggcgtgggagaaccg

SEQ ID NO: 763 - DNA, 28 bp, Artificial
  Synthetic
gtgagtcgccgtgtaggcggttgaaagt

SEQ ID NO: 764 - DNA, 27 bp, Artificial
  Synthetic
gttttcccccgcgcacgcggggacgac

SEQ ID NO: 765 - DNA, 28 bp, Artificial
  Synthetic
gtgtgccgccgtgtaggtggcttgaagt

SEQ ID NO: 766 - DNA, 31 bp, Artificial
  Synthetic
ctgtctcctccgcgcacgcgcgggtgaaccg

SEQ ID NO: 767 - DNA, 26 bp, Artificial
  Synthetic
gccgtcgtttgaggcggcacgacaac

SEQ ID NO: 768 - DNA, 28 bp, Artificial
  Synthetic
ggttcacccccgcgtacgcggggaatac

SEQ ID NO: 769 - DNA, 27 bp, Artificial
  Synthetic
acccgctggctggaagtattccccccg

SEQ ID NO: 770 - DNA, 29 bp, Artificial
  Synthetic
ccgataacccccgcacacaggggaaacac

SEQ ID NO: 771 - DNA, 29 bp, Artificial
  Synthetic
gtctctcccgtgcgcgggagggttaacgg

SEQ ID NO: 772 - DNA, 29 bp, Artificial
  Synthetic
ccgataacccccgcatgcggggggaatac

SEQ ID NO: 773 - DNA, 29 bp, Artificial
  Synthetic
ccactcacccgtgcatgcacgggaaatac

SEQ ID NO: 774 - DNA, 25 bp, Artificial
  Synthetic
gtatgctccgcaggcgcggagatgt

SEQ ID NO: 775 - DNA, 24 bp, Artificial
  Synthetic
gatgatctactatagaattgaaag

SEQ ID NO: 776 - DNA, 40 bp, Artificial
  Synthetic
cggcctcgcaacacgcagaccctactggtctggttgcgag

SEQ ID NO: 777 - DNA, 36 bp, Artificial
  Synthetic
ccagtaagggtagctcagcaccctataagctgggac

SEQ ID NO: 778 - DNA, 24 bp, Artificial
  Synthetic
ctttcaactccatgtaatggattc

SEQ ID NO: 779 - DNA, 30 bp, Artificial
  Synthetic
gtttcaattctcctagagtcttattgcaac

SEQ ID NO: 780 - DNA, 35 bp, Artificial
  Synthetic
gttgcaacagtgcttaccgtttacggtattgaaac

SEQ ID NO: 781 - DNA, 35 bp, Artificial; identical to SEQ ID NO: 780
  Synthetic

SEQ ID NO: 782 - DNA, 29 bp, Artificial
  Synthetic
cttctaaacctacataggatatttcaaac

SEQ ID NO: 783 - DNA, 29 bp, Artificial
  Synthetic
cttctaaacctacataggatatttcaaac

SEQ ID NO: 784 - DNA, 30 bp, Artificial
  Synthetic
gttctcacaacctatagttataatccaaac

SEQ ID NO: 785 - DNA, 29 bp, Artificial
  Synthetic
cttctaaacctacataggatatttcaaac

SEQ ID NO: 786 - DNA, 29 bp, Artificial
  Synthetic
cttctaaacctacataggatatttcaaac

SEQ ID NO: 787 - DNA, 30 bp, Artificial
  Synthetic
gtttagttgctacctttgagggattgaaac

SEQ ID NO: 788 - DNA, 30 bp, Artificial
  Synthetic
gtttttactctacctataagggattgaaac

SEQ ID NO: 789 - DNA, 35 bp, Artificial; identical to SEQ ID NO: 780
  Synthetic

SEQ ID NO: 790 - DNA, 30 bp, Artificial
  Synthetic
gtttcaaagccagatagctaggatgggaag

SEQ ID NO: 791 - DNA, 35 bp, Artificial; identical to SEQ ID NO: 780
  Synthetic

SEQ ID NO: 792 - DNA, 35 bp, Artificial
  Synthetic
gtttcaataccgtaaacggtaagcactgttgcaac

SEQ ID NO: 793 - DNA, 35 bp, Artificial; identical to SEQ ID NO: 780
  Synthetic

SEQ ID NO: 794 - DNA, 29 bp, Artificial
  Synthetic
cttctaaacctacataggatatttcaaac

SEQ ID NO: 795 - DNA, 30 bp, Artificial
  Synthetic
cttttaacccctcacagaaggtatctgcac

SEQ ID NO: 796 - DNA, 36 bp, Artificial
  Synthetic
agtttcaataccgtaaacggtaagcactgttgcaac

SEQ ID NO: 797 - DNA, 35 bp, Artificial; identical to SEQ ID NO: 780
  Synthetic

SEQ ID NO: 798 - DNA, 35 bp, Artificial; identical to SEQ ID NO: 792
  Synthetic

SEQ ID NO: 799 - DNA, 35 bp, Artificial; identical to SEQ ID NO: 792
  Synthetic

SEQ ID NO: 800 - DNA, 37 bp, Artificial
  Synthetic
ctttttgtatcctccaacggatcgaataggttgcaac

SEQ ID NO: 801 - DNA, 35 bp, Artificial; identical to SEQ ID NO: 780
  Synthetic

SEQ ID NO: 802 - DNA, 35 bp, Artificial; identical to SEQ ID NO: 780
  Synthetic

SEQ ID NO: 803 - DNA, 23 bp, Artificial
  Synthetic
gtttccacctataaggggttgta

SEQ ID NO: 804 - DNA, 36 bp, Artificial; identical to SEQ ID NO: 520
  Synthetic

SEQ ID NO: 805 - DNA, 30 bp, Artificial
  Synthetic
gttctaaacttccactacattgtttaaaac

SEQ ID NO: 806 - DNA, 29 bp, Artificial
  Synthetic
gttttataccttatttgaatagttaaaag

SEQ ID NO: 807 - DNA, 30 bp, Artificial; identical to SEQ ID NO: 805
  Synthetic

SEQ ID NO: 808 - DNA, 30 bp, Artificial
  Synthetic
gttttaaacaatgtagtggatgtttagaac

SEQ ID NO: 809 - DNA, 30 bp, Artificial
  Synthetic
gttctaaacttctacgtatagtatctaaac

SEQ ID NO: 810 - DNA, 30 bp, Artificial; identical to SEQ ID NO: 808
  Synthetic

SEQ ID NO: 811 - DNA, 30 bp, Artificial
  Synthetic
gttttaaataatacaatgaaagtttagaac

SEQ ID NO: 812 - DNA, 30 bp, Artificial
  Synthetic
gttttaaataaagtattggaagtttagaac

SEQ ID NO: 813 - DNA, 30 bp, Artificial; identical to SEQ ID NO: 808
  Synthetic

SEQ ID NO: 814 - DNA, 30 bp, Artificial
  Synthetic
gttttaaataaagtattgtaagtttagaac

SEQ ID NO: 815 - DNA, 30 bp, Artificial; identical to SEQ ID NO: 805
  Synthetic

SEQ ID NO: 816 - DNA, 30 bp, Artificial; identical to SEQ ID NO: 805
  Synthetic

SEQ ID NO: 817 - DNA, 30 bp, Artificial
  Synthetic
gttctaaacatccactacattgtttaaaac

SEQ ID NO: 818 - DNA, 30 bp, Artificial
  Synthetic
gtatggatatactacatagaagtttagaac

SEQ ID NO: 819 - DNA, 30 bp, Artificial
  Synthetic
gttctaaacatccactacattatttaaaac

SEQ ID NO: 820 - DNA, 30 bp, Artificial
  Synthetic
gttctaaactttcattgtattatttaaaac

SEQ ID NO: 821 - DNA, 29 bp, Artificial
  Synthetic
ttctaaacatccactacattgtttaaaac

SEQ ID NO: 822 - DNA, 30 bp, Artificial
  Synthetic
gttttaaacaatgtattggaagtttagaac

SEQ ID NO: 823 - DNA, 30 bp, Artificial
  Synthetic
gttctaaaccttcattacattgtttaaaac

SEQ ID NO: 824 - DNA, 30 bp, Artificial
  Synthetic
gttctaaacttccaatactttatttaaaac

SEQ ID NO: 825 - DNA, 30 bp, Artificial; identical to SEQ ID NO: 805
  Synthetic

SEQ ID NO: 826 - DNA, 30 bp, Artificial; identical to SEQ ID NO: 808
  Synthetic

SEQ ID NO: 827 - DNA, 30 bp, Artificial
  Synthetic
gttctaaacttccaatacattgtttaaaac

SEQ ID NO: 828 - DNA, 30 bp, Artificial; identical to SEQ ID NO: 817
  Synthetic

SEQ ID NO: 829 - DNA, 30 bp, Artificial; identical to SEQ ID NO: 812
  Synthetic

SEQ ID NO: 830 - DNA, 36 bp, Artificial
  Synthetic
atctacaatagtagaaattatatggcttttatagcc

SEQ ID NO: 831 - DNA, 36 bp, Artificial
  Synthetic
atctacaatagtagaaattatttaagccttatagcc

SEQ ID NO: 832 - DNA, 36 bp, Artificial
  Synthetic
atctacaatagtagaaattatctttatgttctagac

SEQ ID NO: 833 - DNA, 36 bp, Artificial
  Synthetic
atctgcaaaagcagaaattttcttgtgttactcaac

SEQ ID NO: 834 - DNA, 36 bp, Artificial
  Synthetic
gtctataagacgtatttaatttctacttttgtagat

SEQ ID NO: 835 - DNA, 36 bp, Artificial
  Synthetic
atctacaatagtagaaattattagggattactaacc

SEQ ID NO: 836 - DNA, 36 bp, Artificial
  Synthetic
atctaccgaagtagaaattgttagttactatttgac

SEQ ID NO: 837 - DNA, 33 bp, Artificial
  Synthetic
tctagccacatattaatttctactgttgtagat

SEQ ID NO: 838 - DNA, 36 bp, Artificial
  Synthetic
atctacactagtagaaattatttcttattatttaac

SEQ ID NO: 839 - DNA, 36 bp, Artificial
  Synthetic
atctacaatagtagaaattttctttaacctctaggc

SEQ ID NO: 840 - DNA, 36 bp, Artificial
  Synthetic
atctacaatagtagaaattacattggcttaatagcc

SEQ ID NO: 841 - DNA, 36 bp, Artificial
  Synthetic
ctctaaaacctatataaaatttctacttttgtagat

SEQ ID NO: 842 - DNA, 35 bp, Artificial
  Synthetic
tctaacggacttttaaaatttctacttttgtagat

SEQ ID NO: 843 - DNA, 36 bp, Artificial
  Synthetic
gttgatactgtgagcgaaatgtctactaaggtagat

SEQ ID NO: 844 - DNA, 35 bp, Artificial
  Synthetic
tctgacagtatatgttaatttctactctcgtagat

SEQ ID NO: 845 - DNA, 35 bp, Artificial
  Synthetic
ctttataagaacctaagaatttctactgttgtaga

SEQ ID NO: 846 - DNA, 36 bp, Artificial
  Synthetic
gtctaaagaccgaagtaaatttctactcttgtagat

SEQ ID NO: 847 - DNA, 36 bp, Artificial
  Synthetic
gtttaataaccttaaataatttctacttttgtagat

SEQ ID NO: 848 - DNA, 35 bp, Artificial
  Synthetic
tctaatccatatattagatttctactttcgtagat

SEQ ID NO: 849 - DNA, 35 bp, Artificial
  Synthetic
cttaatagctgatataaatttctactgttgtagat

SEQ ID NO: 850 - DNA, 36 bp, Artificial
  Synthetic
gcctaatacctctataaaatttctacttttgtagat

SEQ ID NO: 851 - DNA, 34 bp, Artificial
  Synthetic
ttaatattattaaagaatttctactgttgtagat

SEQ ID NO: 852 - DNA, 34 bp, Artificial
  Synthetic
atctacactagtagaaattctgaatgagttttag

SEQ ID NO: 853 - DNA, 35 bp, Artificial
  Synthetic
tttaaaaataatatttaatttctactgttgtagat

SEQ ID NO: 854 - DNA, 36 bp, Artificial
  Synthetic
atctacaatagtagaaattaaaaagggtatctagac

SEQ ID NO: 855 - DNA, 35 bp, Artificial
  Synthetic
gtctaaagcactattaaatttctactattgtagat

SEQ ID NO: 856 - DNA, 36 bp, Artificial
  Synthetic
gttataaggaacaaataaatgcctgctgttgcaggc

SEQ ID NO: 857 - DNA, 36 bp, Artificial; identical to SEQ ID NO: 18
  Synthetic

SEQ ID NO: 858 - DNA, 36 bp, Artificial
  Synthetic
gtcggatcgctgagcgagcgatctgagaagtggcac

SEQ ID NO: 859 - DNA, 36 bp, Artificial
  Synthetic
atcgcagtcgccgcaattccggggggcgtgaggcgg

SEQ ID NO: 860 - DNA, 35 bp, Artificial
  Synthetic
tctagacatacaggtggaaaggtgagagtaaagac

SEQ ID NO: 861 - DNA, 25 bp, Artificial
  Synthetic
gtctttactctcacctttccacctg

SEQ ID NO: 862 - DNA, 37 bp, Artificial
  Synthetic
atttgaaggtatctccgataagtaaaacgcatcaaag

SEQ ID NO: 863 - DNA, 25 bp, Artificial
  Synthetic
ttttacataccccctctcatgggat

SEQ ID NO: 864 - DNA, 32 bp, Artificial
  Synthetic
gttgcaacttacgcataggtgtaaaatacgag

SEQ ID NO: 865 - DNA, 32 bp, Artificial
  Synthetic
gttgcaatctgtatgctcaggtatactgcgag

SEQ ID NO: 866 - DNA, 31 bp, Artificial
  Synthetic
gttttgaatgaactatgtagaatgtgaattt

SEQ ID NO: 867 - DNA, 32 bp, Artificial
  Synthetic
gttgcaataaacgtatgtgcatgaggtgtgag

SEQ ID NO: 868 - DNA, 32 bp, Artificial
  Synthetic
gatatatcttgtatgcatatgtaggttgtgag

SEQ ID NO: 869 - DNA, 30 bp, Artificial
  Synthetic
gtttaagaataacaatagttgtatttaaat

SEQ ID NO: 870 - DNA, 30 bp, Artificial
  Synthetic
gttaaaacataacaatagatgtattgaaat

SEQ ID NO: 871 - DNA, 30 bp, Artificial
  Synthetic
gcttaaacatatcctttgttgattctcaac

SEQ ID NO: 872 - DNA, 29 bp, Artificial
  Synthetic
gttttatattaactatgtggaatgtaaat

SEQ ID NO: 873 - DNA, 30 bp, Artificial
  Synthetic
atttgattagaactatattggaatgtaaat

SEQ ID NO: 874 - DNA, 30 bp, Artificial
  Synthetic
gggatcttcctcgtgtgcgtgggaagaaca

SEQ ID NO: 875 - DNA, 29 bp, Artificial
  Synthetic
gtctgccccgcgtgtgcatgggtggttcc

SEQ ID NO: 876 - DNA, 29 bp, Artificial
  Synthetic
gttttatattaactatgtgatatgtaaat

SEQ ID NO: 877 - DNA, 37 bp, Artificial
  Synthetic
gttccaattaatcttaagccctattagggattgaaac

SEQ ID NO: 878 - DNA, 29 bp, Artificial
  Synthetic
gggctcatccccgcgcatacggggaaaac

SEQ ID NO: 879 - DNA, 29 bp, Artificial
  Synthetic
gtttacacactacatagtcattataaaac

SEQ ID NO: 880 - DNA, 29 bp, Artificial
  Synthetic
gtcggccccgcgcacgcggggatgagccc

SEQ ID NO: 881 - DNA, 29 bp, Artificial
  Synthetic
cggtcagcccctgcgcgcccgggccggac

SEQ ID NO: 882 - DNA, 28 bp, Artificial
  Synthetic
ggtcgctccccacgcgtacggaccggac

SEQ ID NO: 883 - DNA, 37 bp, Artificial
  Synthetic
gtttcaatcccattgctaggattcattaataagaaac

SEQ ID NO: 884 - DNA, 37 bp, Artificial
  Synthetic
gttgcagtgcccagctcaggggcttgaagactgagac

SEQ ID NO: 885 - DNA, 36 bp, Artificial
  Synthetic
ggtgcggcagccggtagaactgcggcttccgacacc

SEQ ID NO: 886 - DNA, 36 bp, Artificial
  Synthetic
cctacaatacctaagaaatccgtcctaagttgacgg

SEQ ID NO: 887 - DNA, 25 bp, Artificial
  Synthetic
gtgtcgtaggaagacacattttcta

SEQ ID NO: 888 - DNA, 25 bp, Artificial
  Synthetic
gtttcaataaccattccagctatgg

SEQ ID NO: 889 - DNA, 38 bp, Artificial
  Synthetic
ctttcagcccacctattcgtgggaggctgatcgcaaca

SEQ ID NO: 890 - DNA, 37 bp, Artificial
  Synthetic
ctttcaacccacccagtacctggagggttgttgtcac

SEQ ID NO: 891 - DNA, 37 bp, Artificial
  Synthetic
ctttcaatccctagagaaggtattttcttatttcaac

SEQ ID NO: 892 - DNA, 37 bp, Artificial
  Synthetic
gttgcaaccgtccttccagcaatgggagggttgaaag

SEQ ID NO: 893 - DNA, 37 bp, Artificial
  Synthetic
ctttcaatcgcccccttgccgcaaacctggatgaaac

SEQ ID NO: 894 - DNA, 37 bp, Artificial
  Synthetic
ctttcaacccacccctagccgggatggttgttgaaac

SEQ ID NO: 895 - DNA, 37 bp, Artificial
  Synthetic
ctttcaacccgcctcaagctgggaggggtattgaaac

SEQ ID NO: 896 - DNA, 37 bp, Artificial; identical to SEQ ID NO: 891
  Synthetic

SEQ ID NO: 897 - DNA, 38 bp, Artificial
  Synthetic
agtttcatcaaccctcctgatgtgggatgggttgaaag

SEQ ID NO: 898 - DNA, 37 bp, Artificial
  Synthetic
gtttcaacaaccctcacggtaggcggtgagttgaaag

SEQ ID NO: 899 - DNA, 37 bp, Artificial
  Synthetic
gtcacaacaaccctccaggtactaggcgggttgaaag

SEQ ID NO: 900 - DNA, 37 bp, Artificial
  Synthetic
gtcgcaatcgccctcccagaatagggtgggttgaaag

SEQ ID NO: 901 - DNA, 36 bp, Artificial
  Synthetic
gatatagaccaccccaatatcgaaggggactaaaac

SEQ ID NO: 902 - DNA, 36 bp, Artificial
  Synthetic
gttttagtccccttcgtttttggggttatctatatc

SEQ ID NO: 903 - DNA, 36 bp, Artificial
  Synthetic
gttgtagttccctttcattttgggatcattcacaac

SEQ ID NO: 904 - DNA, 35 bp, Artificial
  Synthetic
gttaatgaaaaaagcccgacatagcgggcaatcac

SEQ ID NO: 905 - DNA, 34 bp, Artificial
  Synthetic
gtaagactacctcaaaacgaaagaggactaaaac

SEQ ID NO: 906 - DNA, 37 bp, Artificial
  Synthetic
gttattgcccattatctcgggctcttctcatttctac

SEQ ID NO: 907 - DNA, 31 bp, Artificial
  Synthetic
cttattgctcactatatcgagctgtattcac

SEQ ID NO: 908 - DNA, 24 bp, Artificial
  Synthetic
ttcgatattggggtagtctatatc

SEQ ID NO: 909 - DNA, 37 bp, Artificial
  Synthetic
gtctcaggcggtcgtcagaaagaccgtcatgcagaac

SEQ ID NO: 910 - DNA, 37 bp, Artificial
  Synthetic
ggttcagtccgccgtcgtcttggcggtgatgtgaggc

SEQ ID NO: 911 - DNA, 36 bp, Artificial
  Synthetic
gttaaaagagaatagcccgacatagtgggcaataac

SEQ ID NO: 912 - DNA, 36 bp, Artificial
  Synthetic
gagaaacatcacccccaaatggagggggactgcacc

SEQ ID NO: 913 - DNA, 36 bp, Artificial
  Synthetic
gttgtgtttacccttcaaaataagggcagttacaac

SEQ ID NO: 914 - DNA, 36 bp, Artificial
  Synthetic
gttgtagatacccttcaaaataagggctttcccaac

SEQ ID NO: 915 - DNA, 36 bp, Artificial
  Synthetic
gttgtgattacccttcaaaatgtgatatttcccaac

SEQ ID NO: 916 - DNA, 36 bp, Artificial
  Synthetic
gttgtcatacccatccaaacgataggcttctacaac

SEQ ID NO: 917 - DNA, 36 bp, Artificial
  Synthetic
gttgtatcagcctttagttggaaaggtagacacaac

SEQ ID NO: 918 - DNA, 36 bp, Artificial
  Synthetic
gttgtttttacctttcaaaaagaaggcagatgcaac

SEQ ID NO: 919 - DNA, 36 bp, Artificial
  Synthetic
gttgtattaacctttagtttgaaaggcaaaaacaac

SEQ ID NO: 920 - DNA, 36 bp, Artificial
  Synthetic
gttgtgtgtacccttcgaatagagggtagatccaac

SEQ ID NO: 921 - DNA, 36 bp, Artificial
  Synthetic
gttgtctccacccttctaactaagggtattcccaac

SEQ ID NO: 922 - DNA, 36 bp, Artificial
  Synthetic
gttgggactgctctcattttgaagggtattcacaac

SEQ ID NO: 923 - DNA, 37 bp, Artificial
  Synthetic
gttggtactgtacacgtttttggtgtgcaatcacaac

SEQ ID NO: 924 - DNA, 36 bp, Artificial
  Synthetic
gttgtagttgctctcggtttggagggtataaacaac

SEQ ID NO: 925 - DNA, 36 bp, Artificial
  Synthetic
gctgttgaagccttcattttgaagggtaattacagc

SEQ ID NO: 926 - DNA, 30 bp, Artificial
  Synthetic
gactaaatccaagtagattggaattttaac

SEQ ID NO: 927 - DNA, 36 bp, Artificial
  Synthetic
gttttagactagtgcaaattcgcaccagtgtagtag

SEQ ID NO: 928 - DNA, 36 bp, Artificial
  Synthetic
gttttaagaccttgtaaatttacaatcctttcaatc

SEQ ID NO: 929 - DNA, 36 bp, Artificial
  Synthetic
gaactacacccgtgcaaaaatgcaggggtctaaaac

SEQ ID NO: 930 - DNA, 90 bp, Artificial
  Synthetic
aucuaaaauuauaaauguaccaaauaauuaaugcucuguaaucauuuaaaaguauuuugaacggaccucuguuugacacgucugaauaac

SEQ ID NO: 931 - DNA, 88 bp, Artificial
  Synthetic
ucagaaugcaucccaacauucuauacacugaaaucauagaaaaucacguuuguggcccgaccaacugcuucggcaugucggguuuuuu

SEQ ID NO: 932 - DNA, 129 bp, Artificial
  Synthetic
uuaauuacauucuuuuaacaacgaagucgccuucgggcgagcugaaaucaauuugauuaaauauuagauccggcuacugaggucuuugaccuuauccggauuaacgaagagccuccgaggaggcuuuuu

SEQ ID NO: 933 - DNA, 111 bp, Artificial
  Synthetic
uuagagaucauaacgcuaugagcuauaggaaaucaccuucgggugagcugaaauccccuaaagcuaagauugaauccggccacuaucuauuaguagauauccggauauucu

SEQ ID NO: 934 - DNA, 100 bp, Artificial
  Synthetic
uaaauuagaaaucaucuaaauuucgauacccugaaaucaacaaaauuaaagauugaaucguuuuucuaugcucgucuuaauagcgagcauauaacgauuu

SEQ ID NO: 935 - DNA, 108 bp, Artificial
  Synthetic
uuguuagaauguucccgcaacacuuuauagcaaauccguucgaugccuugaaaucaucaaaaagauauaauagacccgcccacuguauuguacauggcgggacuuuuu

SEQ ID NO: 936 - DNA, 99 bp, Artificial
  Synthetic
guuuuacuucuuucuaaauaaacauaguuaaguuaaaacaagcuuaaagcgucaauguaauauuuuauuaacacccuacugugucagugggguuuuuuu

SEQ ID NO: 937 - DNA, 154 bp, Artificial
  Synthetic
auuucaaaauauucccccuuuacauuuuucaaaagaaaauguacgcuaagaguguuacuacucuguaacauuacauugguacguuaaaauaagcuuaaagcguaaaaguuggcccuaugaggucuccgccaucgacuucgucgguggcuuuuuu

SEQ ID NO: 938 - DNA, 104 bp, Artificial
  Synthetic
aacuacguuggaacuauucgaaacaacacagccaaaagauuuuucuuuugaguuaaaauaugguuauccauaaucaguuaugcgcaccgauucggugcuuuuuu

SEQ ID NO: 939 - DNA, 90 bp, Artificial
  Synthetic
auuguuaguauucaaaauaacauagcaaguuaaaauaaggcuuuguccguuaucaacuuuuaauuaaguagcgcuguuucggcgcuuuuu

SEQ ID NO: 940 - DNA, 89 bp, Artificial
  Synthetic
guuggaaccauucaaaacagcauagcaaguuaaaauaaggcuaguccguuaucaacuugaaaaaguggcaccgagucggugcuuuuuuu

SEQ ID NO: 941 - DNA, 96 bp, Artificial
  Synthetic
uugugguuugaaaccauucgaaacaacacagcgaguuaaaauaaggcuuaguccguacucaacuugaaaagguggcaccgauucgguguuuuuuuu

SEQ ID NO: 942 - DNA, 102 bp, Artificial
  Synthetic
guuggaaucauucgaaacaacacagcaaguuaaaauaaggcagugauuuuuaauccaguccguacacaacuugaaaaagugcgcaccgauucggugcuuuuu

SEQ ID NO: 943 - DNA, 120 bp, Artificial
  Synthetic
cgucuugauuaccagucaggacagcacugcgagucaaaauacggcuuugccaaacuugccucccuucggaggcgucucguaggagacaauuugaagccccuuuaggggcuucauuuuucu

SEQ ID NO: 944 - DNA, 89 bp, Artificial
  Synthetic
guuuuacuauuucuagauucuuuaagaucuacaaaaauaaggauuuauuccgaauuuaccaccuauuuuaauuaauaggugguuuuuuu

SEQ ID NO: 945 - DNA, 146 bp, Artificial
  Synthetic
uguugagacgacauccucaacaacuugaauugauugaucugacaucuacgaguugagaucaaacaaagcuucagcugaguuucaauuucugagcccauguugggccauacauaugccacccgagugcaaaucggguggcuuuuuuu

SEQ ID NO: 946 - DNA, 144 bp, Artificial
  Synthetic
ggauuguuuggucgcaauccaugaucaaggucauugaccugacaggcauaaauugaaauaaagcaagguuucgaccaagcuucagaagguuuuauaccuggccuuauggcugugaggcucccgauaaugucgggagccucuuuu

SEQ ID NO: 947 - DNA, 167 bp, Artificial
  Synthetic
uugggauugaucaucccaaacaucauuggguucuaccucauugaucugacacacagcauugaaguaaagcaagauuaauuucaagcuuaauuuucuucacauuuuaugugcagaagggcuuaugcccacaauacauaaaaaguccgcauucacuugcggacuuuuau

SEQ ID NO: 948 - DNA, 149 bp, Artificial
  Synthetic
caugguuagcuaccauacaagcaagaauuguuuagcuaacuauucuugcuaggaagaacccauuaaucugacauacaggguuaaaguaacgcaagggcuucagcccaagcuucaugaacuuuuaaaaaguuggccuuauggccuuuuuu

SEQ ID NO: 949 - DNA, 116 bp, Artificial
  Synthetic
uaaucucaguuuaauaccuauaugagauuacaucaugaguucaaauaaaaguuuacucaaaucgcccgaaagagcccacauugguggacuaaacaaaucuucggauuuguuuuuuu

SEQ ID NO: 950 - DNA, 129 bp, Artificial
  Synthetic
auaaguaauccaauuaguuuuggagguuuacagaauuacacuacgaguucaaauacaaauuuauuuacaaugccuucgggccacccgacguaggguaucaucucaauucuucugaauugggauuuuuuu

SEQ ID NO: 951 - DNA, 120 bp, Artificial
  Synthetic
gaaauugucuuauaccaguaagauaauuuacauaguaaguucaaacaagcuuuuagcgaaauuaccgcuuugcggauucacauugugugaaguuaacucucgaaagagaguuuuuucuuu

SEQ ID NO: 952 - DNA, 121 bp, Artificial
  Synthetic
auaucauuaucauugauuuacaaggugaguucaaacaaggauuuauccguaauugauugcucgcauugugcgacauuuucuuauguaaaucgugaagucggacuuucgacuucuuuuuuuu

SEQ ID NO: 953 - DNA, 106 bp, Artificial
  Synthetic
guugacuaccauaugagauuacacuacacgguucaaauaaagaauuuuucuaaucgcccaaugggcccauauugauauggaugaaacucgcuuagcgaguuuuuuu

SEQ ID NO: 954 - DNA, 115 bp, Artificial
  Synthetic
auuuaagauccaucuuaaauuacacaacgaguucaaauaagaauucaucaaaaucgucccuuuugggaccgcucauuguggagcaucaaggcuuaacaugguuaagccuuuuuuu

SEQ ID NO: 955 - DNA, 84 bp, Artificial
  Synthetic
guacuuauaccuaaaauuacagaaucuacugaaacaagacaauaugucguguuuaucccaucaauuuauuggugggauuuuuuu

SEQ ID NO: 956 - DNA, 92 bp, Artificial
  Synthetic
ugaucauaaucuagcaaaaguuuauaugaucuaacaaaacaaggguuuaucccggaaucaaguuccaaguauaugcuuggagcuuuuucuuu

SEQ ID NO: 957 - DNA, 109 bp, Artificial
  Synthetic
uguaagggacgccuuacacaguuacuuaaaucuugcagaagcuacaaagauaaggcuucaugccgaaaucaacacccugucauuuuauggcaggguguuuucguuauuu

SEQ ID NO: 958 - DNA, 130 bp, Artificial
  Synthetic
uguagucgacggacuaccguguuugacgaaacacgucuuuaauaauuuuacugauaagaaauuauugagaaucuacaaaaauaaggcaucuugccgaauuuaccgcccuacauauguagggcgguuuuuu

SEQ ID NO: 959 - DNA, 95 bp, Artificial
  Synthetic
uguagagaaaauuuuauagucacguaaauuuuucagaucuacuaaaacaaggcuuuaugccgaaaucaggagcaccgacgggugcuccuuuuuuu

SEQ ID NO: 960 - DNA, 110 bp, Artificial
  Synthetic
uguauuucgaaauacagauguacaguuaagaauacauaagaaugauacaucacuaaaaaaaggcuuuaugccguaacuacuacuuauuuucaaaauaaguaguuuuuuuu

SEQ ID NO: 961 - DNA, 72 bp, Artificial
  Synthetic
cuaguaagaaauugucgcacaaaaauaagacgcauuaugcugucgaauuuccccaccuagugggguuuuuuu

SEQ ID NO: 962 - DNA, 92 bp, Artificial
  Synthetic
auuauugcuuacacaauuauugucgugcuaaaauaaggcgcuguuaaugcagcugccgcauccgccagagcauuuaugcucuggcuuuuuuu

SEQ ID NO: 963 - DNA, 115 bp, Artificial
  Synthetic
uauauauuacuuacuuaacaaaauaauuguacgauuccaaaauaaggcgcuuauguaagaugcaauaaugcacuuauauaagcugccguaaacgccgagguaacucgguuuuuuu

SEQ ID NO: 964 - DNA, 98 bp, Artificial
  Synthetic
aguacaaauuaauuauuguuuacccaaauauuguacauccuaaaucaaggcgcuuaauugcugccguaauugcugaaagcguagcuuucaguuuuuuu

SEQ ID NO: 965 - DNA, 92 bp, Artificial
  Synthetic
cgucgucgcugcgcgaaauggcugaguaggcagcggcuauaauaagggguguggaggcauccugcgaaguucuacucuacggaguaucuucu

SEQ ID NO: 966 - DNA, 73 bp, Artificial
  Synthetic
aagaaauuuaaaaagggacuaaaauaaagaguuugcgggacucugcgggguuacaauccccuaaaaccgcuuu

SEQ ID NO: 967 - DNA, 96 bp, Artificial
  Synthetic
uaugggaaaucggaagggaagccacggcaaggugguuucauagaaaucacugaaggauuacccucgucacagaaauguggcggggggauuccuauu

SEQ ID NO: 968 - DNA, 125 bp, Artificial
  Synthetic
uaagaucauaucacagcaaugaucuuaggguuacuaugauaagggcuuucuacuuuagggguagagaugucccgcggcguuggggaucgccuauugcccuuaaagggcacuccccauuuuaauuu

SEQ ID NO: 969 - DNA, 107 bp, Artificial
  Synthetic
uuaaucaggaacuagguauagcauaucgagaguuuaacuaguuacuauaacaaggcauuaagccguaaaguauccccuauguucauuugaaccuagggguaucuuuu

SEQ ID NO: 970 - DNA, 100 bp, Artificial
  Synthetic
auggcauaucggagccugaauuguugcuauaauaaggugcuggguuuagcccagaccgccaaguuaaccccggcauuuauugcugggguaucuuguuuuu

SEQ ID NO: 971 - DNA, 103 bp, Artificial
  Synthetic
cgauugugguuauccggggugagagccguugcugcaauaaggaggggucgcaagaccccguccguacccaaaagccuggcagggaaaccugucaggcuuuuuu

SEQ ID NO: 972 - DNA, 111 bp, Artificial
  Synthetic
acauauugucgcacugcgaaaugagaaccguugcuacaauaaggccgucugaaaagaugugccgcaacgcucugccccuuaaagcuucugcuuuaaggggcaucguuuauu

SEQ ID NO: 973 - DNA, 110 bp, Artificial
  Synthetic
gcauauuguugcacugcgaaaugagagacguugcuacaauaaggcuucugaaaagaaugaccguaacgcucugccccuugugauucuuaauugcaaggggcaucguuuuu

SEQ ID NO: 974 - DNA, 99 bp, Artificial
  Synthetic
cuaagagaauuauaucauaccaagugauaauuagguuauuacaauaagguaagaaaccuaaaagcucuaaucccauucuucggaaugggauuaucuuuu

SEQ ID NO: 975 - DNA, 105 bp, Artificial
  Synthetic
gcgagggauaucauaccacaucaaggcuugcgagguugcuaugauaaggcaacaggccgcaaagcacugacccgcauuccaaugaaugcgggucaucuacuuuuu

SEQ ID NO: 976 - DNA, 123 bp, Artificial
  Synthetic
aucacagggugccauuaccagagaugguagcacgguggaacggaccggcaccuacaggacaagugaucauacacgugacagccgccucccccgccccaguggccaaggggaggcggcuuuucu

SEQ ID NO: 977 - DNA, 105 bp, Artificial
  Synthetic
gacuuuccagcagaucgggaauugcgcuuugcuacuaacaagcugaauccguuaggaguaaaugcaccaaaugagagggccggcuuuugccggcccuuugcuuuu

SEQ ID NO: 978 - DNA, 87 bp, Artificial
  Synthetic
cgucuagcaaggaacgcggcguggccucuccguuaacaaggcacaugccaccagaucgaggcgggcuccgguccgccucuuugcuuu

SEQ ID NO: 979 - DNA, 90 bp, Artificial
  Synthetic
cacucaguucacugggauaugcgcucugaccgcuaacaagcugaaagaugcaccaaauggaaagccccgcaugcggggcuuucgucuuuu

SEQ ID NO: 980 - DNA, 109 bp, Artificial
  Synthetic
gccauuaauaauugauugcaauaacacucuggugauugaggagccuaugguuaacaaguggguuuccugcacaaaucuaagagcugccuccgggcggcucuuuugcuuu

SEQ ID NO: 981 - DNA, 93 bp, Artificial
  Synthetic
uggaaauucgauggggaucggggguccagccguuaacauguucccuucggggagcacgaaaugcggggcgggccacgguccgccccuuuuuuu

SEQ ID NO: 982 - DNA, 83 bp, Artificial
  Synthetic
guucagaauucgcggucgagccguuaacaagcucgaagaagcaccacauuaaaacgcguccugcggggcgcguuuucuuuuuu

SEQ ID NO: 983 - DNA, 125 bp, Artificial
  Synthetic
uagcaaaucgagaggcggucgcuuuucgcaagcaaauugaccccuugugcgggcucggcaucccaaggucagcugccgguuauuaucgaaaaggcccaccgcaagcagcgcgugggccuuuuuuu

SEQ ID NO: 984 - DNA, 107 bp, Artificial
  Synthetic
uucugucccauuuguugugauuugcuuuugcacagcauccuuuggacaacuuguucuuugaggauauuaaaaccaaccuaucuguuuaagauagucaauaucuuuuu

SEQ ID NO: 985 - DNA, 158 bp, Artificial
  Synthetic
uuaauguguaaauauaaaaaaauuuaucgaaaaauacaauaguauuaaaaaauuauauguauauuugucaacacaaauuugaaagcaaaucacaauaaggauuauuccguugugaaaacauuuggaagggggaguauuuauacuccucguucuuuuuu

SEQ ID NO: 986 - DNA, 94 bp, Artificial
  Synthetic
cguugauuaaacaaaucaauuuuuacaucuuaucacagcaaggcuauaugccgaaggauguaauccuauacucccgcuucggugggaguuuuuu

SEQ ID NO: 987 - DNA, 112 bp, Artificial
  Synthetic
auguuuuauauauuugcagcaugauuaauauuucuaaucuuuaaucuuaucacaauaaggcuauaugccguagaugaaaaucuuuaguccugcuucggugggacuuuuuuuu

SEQ ID NO: 988 - DNA, 121 bp, Artificial
  Synthetic
guuuguuuuaucagaaauaaguuguauauuugcacucagauacacagugaagacuuuucacaacaaggcuauaagccgaagauuuucuuguacccugcggucaaccacagggucuuuuuuu

SEQ ID NO: 989 - DNA, 80 bp, Artificial
  Synthetic
guuauaucuuaacaaaaaccagcgauuaucucuaauaagacuuaagucgcaaaaugcucccuauuuugggagcuuuuuuu

SEQ ID NO: 990 - DNA, 75 bp, Artificial
  Synthetic
gagacaggcuaccuagcaagaccccuucguggggucgcauucuucacccccucgcagcagcgaggggguucguuu

SEQ ID NO: 991 - DNA, 109 bp, Artificial
  Synthetic
acaaaacaucugaacaucacuuuaacucccaacggauucaagacaaaauuugaaaugcaaaccgauuuuccugacugccagccagucacaccgguaacaaaagcauuuu

SEQ ID NO: 992 - DNA, 100 bp, Artificial
  Synthetic
guuguaacaggguaggguuuuuugaggggucuuaaaaucaagaacuguuacaacaguuccauucuagggcccaucuucggacgggccucagccuuuuuuu

SEQ ID NO: 993 - DNA, 151 bp, Artificial
  Synthetic
gucuaaaggacagaauuuuucaacgggugugccaauggccacuuuccagguggcaaagcccguugaacuucucaaaaagaacgcucgcucaguguucugaccuuucgagcgccuguucagggcgaaaacccugggaggcgcucgaagcaua

SEQ ID NO: 994 - DNA, 159 bp, Artificial
  Synthetic
gucuagaggacagaauuuuucaacgggugugccaauggccacuuuccagguggcaaagcccguugagcuucucaaaaagaacgcucgcucaguguucugaccuuucgagcgccuguucagggcgaaaacccugggaggcgcucgaaucauaggugggac

SEQ ID NO: 995 - DNA, 107 bp, Artificial
  Synthetic
cuggacgaugucucuuuuauuucuuuuuucuuggaucugaguacgagcacccacauuggacauuucgcauggugggugcucguacuauagguaaaacaaaccuuuuu

SEQ ID NO: 996 - DNA, 75 bp, Artificial
  Synthetic
gcuggagacguuuuuugaaacggcgagugcugcggauagcgaguuucucuuggggaggcgcucgcggccacuuuu

SEQ ID NO: 997 - DNA, 94 bp, Artificial
  Synthetic
accauauccagguguugauuguaaacacuuagcgggugaaauuauauauguuuguaauaucuucacuauccaaaguuaucucugguuuugguuu

SEQ ID NO: 998 - DNA, 87 bp, Artificial
  Synthetic
auuuguuuauugaaagaagccuagacguuaggguucgcgugcauguuaggcuccagcagguaccuccguuauaaccuaaccucguuu

SEQ ID NO: 999 - DNA, 80 bp, Artificial
  Synthetic
agucccagcgacuaugucguauggacgaagcgcuuauuuaucggagauagcuccgggugcaaacucggagcuguuuuuuu

SEQ ID NO: 1000 - DNA, 75 bp, Artificial
  Synthetic
ccagcgacuaugucguauggguaaagcgcuuauuuaucggagaguucuggaugcaaacccagagcuguuuuuuua

SEQ ID NO: 1001 - DNA, 122 bp, Artificial
  Synthetic
gagacgcaauaccuugcgaucagagcggcgcgauguauacucgcagcgagacaggcuaccuagcaagaccccuucguggggucgcauucuucacccccucgcagcagcgaggggguucguuu

SEQ ID NO: 1002 - DNA, 104 bp, Artificial
  Synthetic
aauaauaauaaaaaacgaugcccccugaggaagucccaggaggcagagcguugcgagaaauuuaucucuuauuguaacaacguuuuucauuucgcagugccauu

SEQ ID NO: 1003 - DNA, 79 bp, Artificial
  Synthetic
ggauucaagacaaaauuugaaaugcaaaccgauuuuccugacugccagccagucacaccgguaacaaaagcauuuuuuu

SEQ ID NO: 1004 - DNA, 84 bp, Artificial
  Synthetic
gggucacuaauuugaagcaaaucacaauaaggacuauuccguugugaaaacauuuaggauggggcauuugccucguccuuuuuu

SEQ ID NO: 1005 - DNA, 128 bp, Artificial
  Synthetic
ugaucaaggucauugaccugacaggcauaaauugaaauaaagcaagguuucgaccaagcuucagaagguuuuauaccuggccuuauggcugugaggcucccgauaaugucgggagccucuuuucauuu

SEQ ID NO: 1006 - DNA, 136 bp, Artificial
  Synthetic
uggcuuuaucaagaauggugagaccaguccgaugguggaugaggaucugcucuuuuucauuacgaaauccauaaaaaggaaugacaucaugcaauucaacauauccauagcuguauugcaugaugucauuccuuuu

SEQ ID NO: 1007 - DNA, 129 bp, Artificial
  Synthetic
ggugucuagguccuugaccugauaggcauauauugaaauaaagcaagacccuugggucaagcuucagaagguuuuagaccuggccuuauggccauggggcucccggcagcgccgggagccccucuuucu

SEQ ID NO: 1008 - DNA, 90 bp, Artificial
  Synthetic
uauaaucucauaagaaauuuaaaaagggacuaaaauaaagaguuugcgggacucugcgggguuacaauccccuaaaaccgcuuuuaaaau

SEQ ID NO: 1009 - DNA, 82 bp, Artificial
  Synthetic
auuugaaagcaacucacaauaaggauuauuccguugugaaaacaucuaaaggagccccuauaguaauauagggguuguuuuu

SEQ ID NO: 1010 - DNA, 87 bp, Artificial
  Synthetic
uagaacugaaccucaguaagcauuggcucguuuccaauguugauugcuccgccggugcuccuuauuuuuaagggcgccggcuuucuu

SEQ ID NO: 1011 - DNA, 83 bp, Artificial
  Synthetic
uacaauuuugaagcaauucacaauaaggauuauuccguugugaaaacauuuaaagcggccucuaaagggucgcuuuuuuauuu

SEQ ID NO: 1012 - DNA, 85 bp, Artificial
  Synthetic
guagauucaaaauuuacauucaaguucaaauaaagauuuuaucuaaauugccuucgggccuggaucggugucgguccauuuuuuu

SEQ ID NO: 1013 - DNA, 123 bp, Artificial
  Synthetic
ggcaauucgguagggaaaccacgguaacagaauuaccguaagguuuuuucugugaaggaucaucccucgcuugggcaaccaggcgggggaaauuccucguucgggccaaucagcccuucauuu

SEQ ID NO: 1014 - DNA, 109 bp, Artificial
  Synthetic
aguuuuggucuguucaaaauaacacaacgaguuaaaauaaagcuuaugcuuaaaugcugguuauccaguugucauuuagaugacgugcaucacuucggugaugcuuuuu

SEQ ID NO: 1015 - DNA, 126 bp, Artificial
  Synthetic
caucuaaacauuauugauuuaacauacuguguugagaucaaacggagcuuuuuagcgauguuuauccuuuugagccauuauauggccauacauauuauaaaaagugccccuaaaagggcacuuuuu

SEQ ID NO: 1016 - DNA, 104 bp, Artificial
  Synthetic
cuaguggaguuauacaaaacaacauuagcgaaguuaaaauaagguuuaauccguaaucaacaaguuuuaucuugcgcgcgccguuucggcgcuuuuuuuguuuu

SEQ ID NO: 1017 - DNA, 88 bp, Artificial
  Synthetic
uacguggagucauacaaagccacauagcaaguuaaaauaagguuuuauccguaaucaaucaugggcgcuguuucggcgccuauuuuuu

SEQ ID NO: 1018 - DNA, 103 bp, Artificial
  Synthetic
ccuguuuaacaucauugauuuaacauccugguuuaaagucaaacaaggcaguaaugccaaguucuuacuuaugacucacguaugugagcauacauauaaaaaa

SEQ ID NO: 1019 - DNA, 139 bp, Artificial
  Synthetic
auguuugaccuuauugauuuaacauccuguguuaaaaucaagcaaagcgcuuugcgcggaguuucaacuuuugacccauuauaugggcauuacauaagcgaaaggaaccauucuucaguuggagaaugguuccuuuuuu

SEQ ID NO: 1020 - DNA, 102 bp, Artificial
  Synthetic
cggauagacaucauuguuuugaacuacugguuuaaagucaaacaagguagcaauaccaaguucuuacuuaugacccauuuauaugggcauacauacaauaaa

SEQ ID NO: 1021 - DNA, 81 bp, Artificial
  Synthetic
guuuuauugauaggaaaaugauugagaaucuacaaagauaaggcuuuaugccgaaaugacugccuuuauggcaguuuuuuu

SEQ ID NO: 1022 - DNA, 124 bp, Artificial
  Synthetic
uugaacugauugauucgacaucuacacguugagaucaaacaaagcuucggcugaguuucaauuuuugagcccauguugggccauacauaccgccauccgggaagauaccggguggcauuuuuuu

SEQ ID NO: 1023 - DNA, 124 bp, Artificial; identical to SEQ ID NO: 1022
  Synthetic

SEQ ID NO: 1024 - DNA, 109 bp, Artificial
  Synthetic
cguuuugaacugauugauucgacaucuacacguugagaucaaacaaagccucggcugaguuucaauuucugagcccauguugggccauacauaccgccauccgggaaaa

SEQ ID NO: 1025 - DNA, 124 bp, Artificial; identical to SEQ ID NO: 1022
  Synthetic

SEQ ID NO: 1026 - DNA, 124 bp, Artificial; identical to SEQ ID NO: 1022
  Synthetic

SEQ ID NO: 1027 - DNA, 124 bp, Artificial; identical to SEQ ID NO: 1022
  Synthetic

SEQ ID NO: 1028 - DNA, 124 bp, Artificial; identical to SEQ ID NO: 1022
  Synthetic

SEQ ID NO: 1029 - DNA, 121 bp, Artificial
  Synthetic
uugaacugauugauucgacaucuacacguugagaucaaacaaagccucggcugaguuucaauuucugagcccauguugggccauacauaccgccauccgggaaaacccggaugguuuuuuu

SEQ ID NO: 1030 - DNA, 121 bp, Artificial
  Synthetic
uugaacugauugauucgacaucuacacguugagaucaaacaaagcuucggcugaguuucaauuuuugagcccauguugggccauacauacgccauccgggaaaacucggguggcuuuuuuu

SEQ ID NO: 1031 - DNA, 109 bp, Artificial; identical to SEQ ID NO: 1024
  Synthetic

SEQ ID NO: 1032 - DNA, 121 bp, Artificial
  Synthetic
uugaacugauugauucgacaucuacacguugagaucaaacaaagcuucggcugaguuucaauuuuugagcccauguugggccauacauacgccauccgggaaaacccguguggcuuuuuuu

SEQ ID NO: 1033 - DNA, 121 bp, Artificial
  Synthetic
uugaacugauugauucgacaucuacacguugagaucaaacaaagcuucggcugaguuucaauuuuugagcccauguugggccauacauacgccauccgggaaaacccggguggcuuuuuuu

SEQ ID NO: 1034 - DNA, 124 bp, Artificial; identical to SEQ ID NO: 1022
  Synthetic

SEQ ID NO: 1035 - DNA, 124 bp, Artificial; identical to SEQ ID NO: 1022
  Synthetic

SEQ ID NO: 1036 - DNA, 102 bp, Artificial
  Synthetic
guuuuaagaccuaucgagaacaacacacuuaaguucaaauaaaaauuuauucuauugaccagaaauggcagcccgcugugggcauuuuaacuucauuuuuuu

SEQ ID NO: 1037 - DNA, 98 bp, Artificial
  Synthetic
aaucuugaaucuauugaucugacauacagcgucagaauaaaacaaggccaucggccaaguuuacagcgcucuuuuugagaguggggcuuaugcccaua

SEQ ID NO: 1038 - DNA, 67 bp, Artificial
  Synthetic
uaucuuaacuuuauugaaaugacacucuguuaaaaucaaacaaggcuucggccaaguuuucccuuuu

SEQ ID NO: 1039 - DNA, 113 bp, Artificial
  Synthetic
cuaucuaaacuuauugauuuaacacucugaguuaaaaucaaacaaggcguaagccaaguuucaucuugugagucccuauggaccauaaaggcgcuauugaucuucgugaucaa

SEQ ID NO: 1040 - DNA, 112 bp, Artificial
  Synthetic
acucuuaaccuuauugaucuaacaaucauaauuuaaaaucaagcaaugcguuuuaguacgcagaguuucaacacuugucccgagcuaucgagggacuuuuuuuauguaaaaa

SEQ ID NO: 1041 - DNA, 113 bp, Artificial
  Synthetic
aucgaaauacucauugauuugauacucugaguuaaaaucaaacaaggucuucggaccaaguuuucuauucuaagccucuuaugaggccuuuuuuuaugccuucaaaccaaaaa

SEQ ID NO: 1042 - DNA, 113 bp, Artificial; identical to SEQ ID NO: 1041
  Synthetic

SEQ ID NO: 1043 - DNA, 96 bp, Artificial
  Synthetic
guuaaaccuuauugaauagacaaccugauuuaaaaucaagcaaagcucuucggaguggaguuuauacauuugucccgcguauugcgggcuuuuuuu

SEQ ID NO: 1044 - DNA, 109 bp, Artificial
  Synthetic
acguuugaccuuauugaauagacaaccugauuuaaaaucaagcaaagcucuucggagcggaguuuauacauuugucccguguauugcgggcuuuuuuuaugcaacaaaa

SEQ ID NO: 1045 - DNA, 97 bp, Artificial
  Synthetic
acguuagaccuuauugaauagacaaccugauuuaaaaucaagcaaagcucuucggaguggaguuuauacauuugucccgcguauugcgggcuuuuuu

SEQ ID NO: 1046 - DNA, 97 bp, Artificial
  Synthetic
ugggaugucauugucugaaucaaugauugaaaaauauaacgcucaccgugaucacuugaagcuacucaaacagcucgaggauaagauugacgauccg

SEQ ID NO: 1047 - DNA, 90 bp, Artificial
  Synthetic
guuuuacuauuucuagauucuuuaagaucuacaaaaauaaggauuuauuccgaauuuaccaccuauuuuaauuaauaggugguuuuuuuu

SEQ ID NO: 1048 - DNA, 102 bp, Artificial
  Synthetic
gguucguaaguaaauagaaagacauacuuagauuaaaauaaacaauaauguuaaauggucguaaggcugcucccgcgugggggcuuuuuuuuugccuuuuuu

SEQ ID NO: 1049 - DNA, 106 bp, Artificial
  Synthetic
acauucgagacuacugaucuaacacucauagucaagaucaaacgagugguuuuccacgaguuauuacuuuugagccauguuauucuggccauacauauaauaaaaa

SEQ ID NO: 1050 - DNA, 106 bp, Artificial; identical to SEQ ID NO: 1049
  Synthetic

SEQ ID NO: 1051 - DNA, 168 bp, Artificial
  Synthetic
guucuuaacauuauucaaaugacauacugaguugaaaucaaacaaggauuuauuccaaguuuuauauuuugaaccucuuuuugaggucagaccucuuucuuaauugaaagagguuuuuuuguugcaauuuuuaacaauauuuguaauaguagugguagaaguuuguuu

SEQ ID NO: 1052 - DNA, 125 bp, Artificial
  Synthetic
uucauuaaccuuauugauuugacauacugaguuaaaauaaaacaaugcguaagcaaaguuuacacuuauuagccauuuauaguuggcaauacauaaagaaagaccuaggucauaugaccuagguc

SEQ ID NO: 1053 - DNA, 106 bp, Artificial
  Synthetic
accgauaaacucauugauuugauacucuggguuaaaaucaaacgaaaccuucggguuaaguuuuuacuucugagccucguaugaggccuuuuuuuaugccuucauu

SEQ ID NO: 1054 - DNA, 90 bp, Artificial; identical to SEQ ID NO: 1047
  Synthetic

SEQ ID NO: 1055 - DNA, 112 bp, Artificial
  Synthetic
acacuaaacuuuauugaucuaacaaccagauuuaaaaucaagcaaugcaucuuuugaugcaaaguuucaauacuugucccgagcuaucgagggacuuuuuuuaugcaaaaaa

SEQ ID NO: 1056 - DNA, 92 bp, Artificial
  Synthetic
gaaauacucauugauuugauacucugaguuaaaaucaaacaaggucuuuggaccaaguuuucuauucugagccucuuaugaggccuuuuuuu

SEQ ID NO: 1057 - DNA, 138 bp, Artificial
  Synthetic
ccgcuugaucuuacugaauugucacuaagaguuaaagucagacaaggugguuuaccaccaaguuuuaucuuaugagcccguuaucgagccauacauaucuuguuagaguccuuugcugaaugcgucagcgaaggacuc

SEQ ID NO: 1058 - DNA, 112 bp, Artificial
  Synthetic
acucuaaacuuuauugaucuaacaaccagauuuaaaaucaagcaaugcaucuuuugaugcaaaguuucaacacuugucccgagcuaucgagggacuuuuuuuauacaaaaaa

SEQ ID NO: 1059 - DNA, 112 bp, Artificial; identical to SEQ ID NO: 1058
  Synthetic

SEQ ID NO: 1060 - DNA, 83 bp, Artificial
  Synthetic
guuuuguugauaggaaaaugauugagaaucuacaaagauaaggcuuuaugccgaaauuacugccuuuuuggcaguuuuuuuuu

SEQ ID NO: 1061 - DNA, 141 bp, Artificial
  Synthetic
ggauuaacauuauugguuuaacgucuuguuuuaaaaucaaacaaaacacuuuaguguugaguuuuaacuuuugacccacuuuauuagugggcaguacauaaaaaaggaaucacucuuauaaugaggagugauuccuuuuuu

SEQ ID NO: 1062 - DNA, 115 bp, Artificial
  Synthetic
acauuucaauuuacugauuuaacaaccuuauuuuuaaaaucaagcaaggcuuucgggccgaguuuucacauguguaccgcuuauagcggucuuuuuuuuugcaguaaauaaaaaa

SEQ ID NO: 1063 - DNA, 100 bp, Artificial
  Synthetic
acauuucaauuuacugauuuaacaaccuuauuuuuaaaaucaagcaaggcuuucgggccgaguuuucacauguguaccgcuuauagcggucuuuuuuuuu

SEQ ID NO: 1064 - DNA, 112 bp, Artificial
  Synthetic
caucuaaacuuuauugaucuaacaaccagauuuaaaaucaagcaaugcaucuuuugaugcaaaguuucaauauuugucccacguuaucgagggacuuuuuuuauacaaaaaa

SEQ ID NO: 1065 - DNA, 94 bp, Artificial
  Synthetic
cauuuuaaucucauuagaaugacguacugaguuaaaaucaaacaaagccuuaugcugaguuuuaucuuuugagcccuuaagggccauacauaua

SEQ ID NO: 1066 - DNA, 82 bp, Artificial
  Synthetic
guuuuauugauaggaaaaugauugagaaucugcaaagauaaggcuuuaugccgaaauuacugccucuuaggcaguuuuuuuu

SEQ ID NO: 1067 - DNA, 92 bp, Artificial
  Synthetic
gcucuuaaguaauugagaugacuuacugaguuaaaauaaacauuaaaguuaaaugaccuucuggucggcucccgcgugggggcuauuuuuuu

SEQ ID NO: 1068 - DNA, 104 bp, Artificial
  Synthetic
aggaauaacaucauugauuuaacauccagaguuaaagucaaacaaugcauucugcaaaguuauuucuucugacccauuuauuaaugggcaauacauauuaaaaa

SEQ ID NO: 1069 - DNA, 104 bp, Artificial; identical to SEQ ID NO: 1068
  Synthetic

SEQ ID NO: 1070 - DNA, 140 bp, Artificial
  Synthetic
cuguuugaccuuauagauuuaccacucagaguugaaaucaaacaagaugcuuuagcaucaaguuucccauugugagcccguuaucggugccauacauauuaaaacgaccaagccaccuuuucgugacuuggucguuuuuu

SEQ ID NO: 1071 - DNA, 90 bp, Artificial
  Synthetic
guuuuacuguuucuaaaucuuugaagaucuacaaaaauaaggauuuauuccgaauuuaccaccuauuuuaaaaaauaggugguuuuuuuu

SEQ ID NO: 1072 - DNA, 96 bp, Artificial
  Synthetic
guuuuacuguuacuaaauucuugagaaccuacuaaaauaaggauuuauuccgaauuuaccaccuauuuuuuaauaggugguuuuuuuuuacaaaaa

SEQ ID NO: 1073 - DNA, 96 bp, Artificial
  Synthetic
uuguucaaacuacaugaaaugacguacugaguuaaaaucaacauuuauguuaaauagucucaauugacuggcucccgcgugggggcuauuuuuuuu

SEQ ID NO: 1074 - DNA, 136 bp, Artificial
  Synthetic
cacaucgaauucauugauuugauacucugaguuaaaaucaagcaaggcgcaagccaaguuuaaucuucugaguccuguaugggaccauacauaaagagcguuauugauuucauuaucaauaacgcucuauuuuauu

SEQ ID NO: 1075 - DNA, 140 bp, Artificial
  Synthetic
auguuugaccuuauugauuuaacauccuguguuaaaaucaagcaaugcgcuuugcgcggaguuuccacuuuugacccauuauaugggcauuacauaagcuaaaggaaccauucuucgauuuggagaaugguuccuuuuuu

SEQ ID NO: 1076 - DNA, 103 bp, Artificial
  Synthetic
auguuugaccuuauugauuuaacauccuguguuaaaaucaagcaaugcgcuuugcgcggaguuuccacuuuugacccauuauaugggcauuacauaagcuaaa

SEQ ID NO: 1077 - DNA, 110 bp, Artificial
  Synthetic
auuauaacaucuauguauuugaaauguauuaaugcaucaaaaucuuagaucacaauuucuauaauauacguaaaacagcaaauuuuacuuuauaagaauuuuuauauuuu

SEQ ID NO: 1078 - DNA, 109 bp, Artificial; identical to SEQ ID NO: 1024
  Synthetic

SEQ ID NO: 1079 - DNA, 108 bp, Artificial
  Synthetic
cguuuugaacugauugauucgacaucuacacguugagaucaaacaaagcuucggcugaguuucaauuuuugagcccauguugggccauacauacgccauccgggaaaa

SEQ ID NO: 1080 - DNA, 69 bp, Artificial
  Synthetic
acuuaauaaccucauugauuugauauucagaguuaaaaucaaacaaggcuuaugccaaguuuucucuuu

SEQ ID NO: 1081 - DNA, 140 bp, Artificial
  Synthetic
auguuugaccuuauugauuuaacauccuguguuaaaaucaagcaaugcgcuuugcgcggaguuuccacuuuugacccauuauaugggcauuacauaagcgaaaggaaccauucuucgauuuggagaaugguuccuuuuuu

SEQ ID NO: 1082 - DNA, 117 bp, Artificial
  Synthetic
guucuaaaccuucuugauaugacuacuagcgucaagaucaaacaaggcauuuugccgaguuucaccuuuugaguccguauucggaccauuacauauguaucaagaugucgcccauuu

SEQ ID NO: 1083 - DNA, 109 bp, Artificial
  Synthetic
acacuugaccuuauugauuuaauguccuuuauuugaaaucaaacaacaccauucauggugaaguuuucacaccuguccauccuauauauauggacaccuacauaaaaaa

SEQ ID NO: 1084 - DNA, 117 bp, Artificial; identical to SEQ ID NO: 1082
  Synthetic

SEQ ID NO: 1085 - DNA, 103 bp, Artificial
  Synthetic
guucuaaaccuucuugauaugacuacuagcgucaagaucaaacaaggcauuuugccgaguuucaccuuuugaguccguauucggaccauuacauauguaucaa

SEQ ID NO: 1086 - DNA, 106 bp, Artificial
  Synthetic
acaaucucaacauuucaauuuacugauuuaacaaccugauuuaaaaucaagcaaggucuucggaccgaguuuucacauuuguaccgcuuauagcggucuuuuuuuu

SEQ ID NO: 1087 - DNA, 93 bp, Artificial
  Synthetic
auugaaagauucauugauuugauacucugaguuaaaaucaaacaaaccuucgggugaguuuuuacuucugagccucguauggggccuuuuuuu

SEQ ID NO: 1088 - DNA, 109 bp, Artificial
  Synthetic
cuacuaaauauuauugauuugaaguacagaguuaaaaucaaacaagugcuucagcacaaguuucuacuuuugagucaguuaucugaccaauacauaaaagggaauuaaa

SEQ ID NO: 1089 - DNA, 110 bp, Artificial
  Synthetic
caacuugaauugauugaucugacaucuacgaguugagaucaaacaaagcuucagcugaguuucaauuucugagcccauguugggccauacauaugccacccgagugcaaa

SEQ ID NO: 1090 - DNA, 110 bp, Artificial; identical to SEQ ID NO: 1089
  Synthetic

SEQ ID NO: 1091 - DNA, 143 bp, Artificial
  Synthetic
acuuuugacccuauugaucugacauacagcguuaaaaucaagcaaggcuuucgagccaaguuuaaaucuuuggguucgcuauucggaccguacauaguaaaagggcgucaucgaguucaaauucgauggcgcccuuuaguuuu

SEQ ID NO: 1092 - DNA, 102 bp, Artificial
  Synthetic
acuuuugacccuauugaucugacauacagcguuaaaaucaagcaaggcuuucgagccaaguuuaaaucuuuggguucgcuauucggaccguacauaguaaaa

SEQ ID NO: 1093 - DNA, 103 bp, Artificial
  Synthetic
acguaaaacaucauugauaugacauccagaaguugaaaucaagcaagacuuaaagucaagcuucuacuuuugauucacguuuuguggacaguacauauugaca

SEQ ID NO: 1094 - DNA, 95 bp, Artificial
  Synthetic
accgaaauacucauugauuugauacucugaguuaaaaucaaacaaggucuuuugaccaaguuuucuauucugagccucuuaugaggccuuuuuuu

SEQ ID NO: 1095 - DNA, 119 bp, Artificial
  Synthetic
gguuuugaccucauugauuuaacauucugaguugaaaucaaacaaggcuuuuauagcugaguuuuucuuuuugauccauauauauggacuauacauaaaaaggagucaucugacuccuu

SEQ ID NO: 1096 - DNA, 99 bp, Artificial
  Synthetic
auuuuaaucucauuagaaugacguacugaguuaaaaucaaacaaggucuugugccaaguuuuaucuucugagcccuuaagggccauacauauaagaaaa

SEQ ID NO: 1097 - DNA, 99 bp, Artificial; identical to SEQ ID NO: 1096
  Synthetic

SEQ ID NO: 1098 - DNA, 128 bp, Artificial
  Synthetic
acguuuaaccuauuugaucugacauucaagguuaaaaucaaacaaggcuucggccaaguuuuauucuuagggcucgcuaucgagccauacauaaaaacacacccguuucggcggguguguuuucuuuu

SEQ ID NO: 1099 - DNA, 129 bp, Artificial
  Synthetic
acgcuugacucuauugauuugucacugaaaguuaaaaucaagcaaggccuugugccaaguuuuacuuuuugagcccgguaucgagccauacauagucggcccuuccagucauguggaagggccguuuuu

SEQ ID NO: 1100 - DNA, 86 bp, Artificial
  Synthetic
guuuuacuauuacuaaauucuuaaggaucuacaaaaauaaggauuuauuccgaauuuaccaccuacuuguuaggugguuuuuuuuu

SEQ ID NO: 1101 - DNA, 167 bp, Artificial
  Synthetic
auguucuaaguaauugagaugacuuagagaauuaaaaucaacauuaaaguaaaucgaucauuuugauugggcccaauggggccuauuuuuuugcucugaauuuuuagucgauauguuuaauugauuuaaauaauccgguuggugauucauaaauaaaaucaguuuuu

SEQ ID NO: 1102 - DNA, 143 bp, Artificial
  Synthetic
cugcuugaccuucuagauuuaccacucaguguuaaaaucaaacaagauacugcgguaucaaguuuuccuuuuugagcucguuaucgagccaauacauaauuaaacgaccaacucacuggcaguccaaugaguuggucauuuuu

SEQ ID NO: 1103 - DNA, 121 bp, Artificial; identical to SEQ ID NO: 1029
  Synthetic

SEQ ID NO: 1104 - DNA, 140 bp, Artificial
  Synthetic
gguuuaaccucauugaucugauacacaauauugaaguaacgcaaggcuuuaugccaagcuuaauuacucaucaagaguggccuuauggccguacauaaaaucccguguuguuacaaaacagcacgggauuucuuuuuauu

SEQ ID NO: 1105 - DNA, 145 bp, Artificial
  Synthetic
guauagaaaccauugaucugacauacaaaguugaaguaacgcaaggcgucagccaagcuuauugacucuuuacgaguggcccuauggccguacauaaaaucccguguugccuuugcaacacgggauuuaucuuuauucauccuuu

SEQ ID NO: 1106 - DNA, 91 bp, Artificial
  Synthetic
uuguuaguauucaaaauaacauagcaaguuaaaauaaggcuuuguccguuaucaacuuuuaauuaaguagcgcuguuucggcgcuuuuuuu

SEQ ID NO: 1107 - DNA, 94 bp, Artificial
  Synthetic
auuauugcuuacacaauuauugucgugcuaaaauaaggcgcuguuaaugcagcugccgcauccgccagagcauuuaugcucuggcuuuuuauuu

SEQ ID NO: 1108 - DNA, 89 bp, Artificial
  Synthetic
auugucgcacugcgaaaugagaaccguugcuacaauaaggccgucugaaaagaugugccgcaacgcucugccccuuaaagcuucugcuu

SEQ ID NO: 1109 - DNA, 143 bp, Artificial
  Synthetic
ggguucuaccucauugaucugacacacagcauugaaguaaagcaagauuaauuucaagcuuaauuuucuucacauuuuaugugcagaagggcuuaugcccacaauacauaaaaaguccgcauucacuugcggacuuuuauuuu

SEQ ID NO: 1110 - DNA, 143 bp, Artificial
  Synthetic
uauuuuaacuauauugauuugacgcucugaguuaaaaucaaacaaagcuucggcugaguuuuaucuuuugaggccgcuaucggcccauacauauuuuauucaagaaaagcuauugacgguuaucaaaccaccaauagcuuuuu

SEQ ID NO: 1111 - DNA, 140 bp, Artificial
  Synthetic
acuuuuaaccuuauugauuuaacauccugauuugaaaucaaacaaugcacuuugugcggaguuuauccauuugucccagcuaucugggcaauacauagugaaagaggaaccgacccuuaaugggacggcuccucuuuuuu

SEQ ID NO: 1112 - DNA, 140 bp, Artificial; identical to SEQ ID NO: 1111
  Synthetic

SEQ ID NO: 1113 - DNA, 149 bp, Artificial
  Synthetic
uauuuuaacuauauugauuugacgcucuggguuaaaaucaaacaaagcuuuaugcugaguuuuaucuuuugaguccgcuaucggaccauacauaauuuuauuaagaaaagcuauugaagguuauuaaaccgacaauagcuuuucuuuuu

SEQ ID NO: 1114 - DNA, 137 bp, Artificial
  Synthetic
acaauugaauuuacuucuuuaacauccuaauuugaaaucaagcaaugcacuucggugcggaguuuacacauuugucgucuuuauaggcgcguacauaaagaaaagaggaacguuccuuaauggaaguuccucuuuuu

SEQ ID NO: 1115 - DNA, 79 bp, Artificial
  Synthetic
uacuaauuuuaaaguauucacaauaaggauuuuuccguugugagaacauuagguugccucguccuuauaacggggcuuu

SEQ ID NO: 1116 - DNA, 74 bp, Artificial
  Synthetic
agauuuuuaagcaagucacaauaaggauuauuccguugugaaaacaaacagagcggggcaacucgcucuuuuuu

SEQ ID NO: 1117 - DNA, 78 bp, Artificial
  Synthetic
auaccuaaaauuacagaaucuacuaaaacaaggcaaaaugccguguuuaucucgucaacuuguuggcgagauuuuuuu

SEQ ID NO: 1118 - DNA, 95 bp, Artificial
  Synthetic
cuuguacuuauaccuaaaauuacagaaucuacugaaacaagacaauaugucguguuuaucccaucaauuuauuggugggauuuuuuuauguuuuu

SEQ ID NO: 1119 - DNA, 119 bp, Artificial
  Synthetic
uuguuggaacuauucgaaacagcacagcgcguuaaaauaaggcagugauuuuuuaauccaguccguauucagcuugaaaaagugagcaccgauucggugcuuuuuuuuaugucauuguu

SEQ ID NO: 1120 - DNA, 119 bp, Artificial
  Synthetic
uuguuggagcuauucgaaacaacacagcgaguuaaaauaaggcuuuguccguacacaacuuguaaaaguggcacccgauucgggugcguuuuuuugugcccuuacaaauaauuucaaca

SEQ ID NO: 1121 - DNA, 102 bp, Artificial
  Synthetic
cuugcacaguuacuuaaaucuugcugagccuacaaagauaaggcuucaugccgaaaucaagcacccccguuuuuaacgaggggugcuuuucguuauguaaaa

SEQ ID NO: 1122 - DNA, 110 bp, Artificial
  Synthetic
cuuacacaguuacuuaaaucuugcagaagcuacaaagauaaggcuucaugccgaaaucaacacccuaucuauuauaagauaggguguuuucguuuuauagaggagauuuu

SEQ ID NO: 1123 - DNA, 119 bp, Artificial
  Synthetic
uuguuggaacuauucgaaacaacacagcaaguuaaaauaaggcuuuguccguacacaacuugaaaaagugcgcaccgauucggugcuuuuuauuuuauuauacuaacucauuguacucc

SEQ ID NO: 1124 - DNA, 119 bp, Artificial
  Synthetic
uuguuggaaccauucaaaacagcauagcaaaguuaaaauaaggcuaguccguuaucaacuugaaaaaguggcaccgagucggugcuuuuuuugauacuucuauucuacucugacugcaa

SEQ ID NO: 1125 - DNA, 85 bp, Artificial
  Synthetic
auucacauuauucaaguuuacuacagauucuguauuaagauacaaaaucguaagacuggcugccuuaagcagcuuuuuuuauuuu

SEQ ID NO: 1126 - DNA, 105 bp, Artificial
  Synthetic
cuuacacaguuacuuaaaucuugcagaagcuacaaagauaaggcuucaugccgaaaucaacacccugucuaugacgggguguuuucguguuguaaaggaguuuuu

SEQ ID NO: 1127 - DNA, 119 bp, Artificial
  Synthetic
auguuggaaucauucgaaacaacacagcaaguuaaaauaaggcuuuguccguacucaacuuaaaaagugcgcaccgauucggugcuuuuuuauuucaaucaagaacacaaaaaaacagc

SEQ ID NO: 1128 - DNA, 119 bp, Artificial
  Synthetic
uuguuggaaccauucaaaacaacguagcaaguuaaaauaaggcuaguccguuaucaacuugaaaaaguggcaccgagucggugcuuuuuguaucuuuauuuuaccuugucaauaaacua

SEQ ID NO: 1129 - DNA, 119 bp, Artificial
  Synthetic
ccuauggaacuauucaauacagcauagcaaaguuaaaauaaggcuuuguccguaaucaaccugaaaaggggagcaccgaaucggugcuuuuuuacuuucuaccaacucuaccaauacuu

SEQ ID NO: 1130 - DNA, 99 bp, Artificial
  Synthetic
cuuacacaguuacuuaaaucuugcagaagcuacaaagauaaggcuucaugccgaauucaacacccugucauuuauggcgggguguuuucgauuuguaaa

SEQ ID NO: 1131 - DNA, 109 bp, Artificial
  Synthetic
cuugcacgguuacuuaaaucuugcagagccuacaaagauaaggcuucaugccgaauucaagcaccccauguuuacauggggugcuuuucguuguguaaaaaggagagua

SEQ ID NO: 1132 - DNA, 106 bp, Artificial
  Synthetic
cuuacacaguuacuuaaaucuugcagaagcuacaaagauaaggcuucaugccgaaaucaacacccugucuaugacgggguguuuucguguuguaaaggaguuuuua

SEQ ID NO: 1133 - DNA, 119 bp, Artificial
  Synthetic
uuguuggaacuauucgaaacaacacagugaguuaaaauaaggcuuuguccguacacaacuuauaaaaguggcacccgauucggaugcauuuuuuauuugugauucaaauagauauaaca

SEQ ID NO: 1134 - DNA, 119 bp, Artificial
  Synthetic
ucguuggaacuauucgaaacaacacagcaaaguuaaaauaaggcuuuguccguacacaacuugaaaaagugcgcaccgauucggugcuuuuuuguuuuacuauacuaauuuauucuacu

SEQ ID NO: 1135 - DNA, 119 bp, Artificial
  Synthetic
guguuggaaucauucgaaacaacacagcaaguuaaaauaaggcagugauuuuuaauccaguccguacacaacuugaaaaagugcgcaccgauucggugcuuuuuuauuugcuuuacugu

SEQ ID NO: 1136 - DNA, 100 bp, Artificial
  Synthetic
cuugcacaguuacuuaaaucuugcagagccuacaaagauaaggcuuuaugccgaaaucaagcaccccguuuauacgaggugcuuuucguuuuaaauaaaa

SEQ ID NO: 1137 - DNA, 99 bp, Artificial
  Synthetic
cuuacacaguuacuuaaaucuugcagaagcuacaaagauaaggcuucaugccgaauucaacacccugucauuuauggcgggguguuuucguuuuguaaa

SEQ ID NO: 1138 - DNA, 119 bp, Artificial
  Synthetic
gguuugaaaccauucgaaacaauacagcaaaguuaaaauaaggcuuaguccguauucaacuugagaaaguggcaccgauucggugcuuuuuugcugcacaaagagucccuuggaauuau

SEQ ID NO: 1139 - DNA, 119 bp, Artificial
  Synthetic
uuguuggaacuauucgaaacaacacagcaaguuaaaauaaggcuuuguccguacacaacuugaaaaagugcgcaccgauucggugcuuuuuuaauuacuacaggugcuuuuuuaauuac

SEQ ID NO: 1140 - DNA, 100 bp, Artificial
  Synthetic
cuuacacaguuacuuaaaucuugcagaagcuacaaagauaaggcuucaugccgaaaucaacacccugucauuuuauggcaggguguuuucguuauuuaaa

SEQ ID NO: 1141 - DNA, 119 bp, Artificial
  Synthetic
gguuugaaaccauucgaaacaacacagcgaguuaaaauaaggcuuaguccguacucaacuugaaaagguggcaccgauucgguguuuuuuuuauacacaaagagucccuuggaaucauc

SEQ ID NO: 1142 - DNA, 102 bp, Artificial
  Synthetic
cuuacacaguuacuuaaaucuugcagaagcuacaaagauaaggcuucaugccgaaaucaacacccugucauuuuauggcaggguguuuucguuauuuaaaaa

SEQ ID NO: 1143 - DNA, 79 bp, Artificial
  Synthetic
cucacugggauaugcgcucuggacgcuaacaagccgagagaugcggcgccaaauggaaagccccgcagugcggggcuuu

SEQ ID NO: 1144 - DNA, 108 bp, Artificial
  Synthetic
uuauagccgcugccuacucagccauuucgcgcagcgacgacgauuauauguuagauuuuuuuaaaaaauacuaaaauuuggagcgaagcuuaugauugaacggauuuu

SEQ ID NO: 1145 - DNA, 135 bp, Artificial
  Synthetic
accaaggttgttctaataaaatattgaaaatattgtttattagttcctctttaagtttctttaaatagtcggtattttgttatgaattatgatatcttactattgttttatttgatgaaaaattttcttaaaaca

SEQ ID NO: 1146 - protein, 328 aa, Pseudomonas aeruginosa
GSFTMDDISPSELKTILHSKRANLYYLQHCRVLVNGGRVEYVTDEGRHSHYWNIPIANTTSLLLGTGTSITQAAMRELARAGVLVGFCGGGGTPLFSANEVDVEVSWLTPQSEYRPTEYLQRWVGFWFDEEKRLVAARHFQRARLERIRHSWLEDRVLRDAGFAVDATALAVAVEDSARALEQAPNHEHLLTEEARLSKRLFKLAAQATRYGEFVRAKRGSGGDPANRFLDHGNYLAYGLAATATWVLGIPHGLAVLHGKTRRGGLVFDVADLIKDSLILPQAFLSAMRGDEEQDFRQACLDNLSRAQALDFMIDTLKDVAQRSTVSA

SEQ ID NO: 1147 - protein, 326 aa, Thermotoga maritima
RELYFQGMESVYLFSSGTLKRKANTICLETESGRKYIPVENVMDIKVFGEVDLNKRFLEFLSQKRIPIHFFNREGYYVGTFYPREYLNSGFLILKQAEHYINQEKRMLIAREIVSRSFQNMVDFLKKRKVRADSLTRYKKKAEEASNVSELMGIEGNAREEYYSMIDSLVSDERFRIEKRTRRPPKNFANTLISFGNSLLYTTVLSLIYQTHLDPRIGYLHETNFRRFSLNLDIAELFKPAVVDRLFLNLVNTRQINEKHFDEISEGLMLNDEGKSLFVKNYEQALRETVFHKKLNRYVSMRSLIKMELHKLEKHLIGEQVFGSEE

SEQ ID NO: 1148 - protein, 315 aa, Aquifex aeolicus
GRVYYINSHGTLSRHENTLRFENAEVKKDIPVEDVEEIFVFAELSLNTKLLNFLASKGIPLHFFNYYGYYTGTFYPRESSVSGHLLIKQVEHYLDAQKRLYLAKSFVIGSILNLEYVYKISADTYLNKVKETNSIPELMSVEAEFRKLCYKKLEEVTGWELEKRTKRPPQNPLNALISFGNSLTYAKVLGEIYKTQLNPTVSYLHEPSTKRFSLSLDVAEVFKPIFVDNLIIRLIQENKIDKTHFSTELNMTFLNEIGRKVFLKAFNELLETTIFYPKLNRKVSHRTLIKLELYKLIKHLLEEEVYLPLNYGGLK

SEQ ID NO: 1149 - protein, 101 aa, Sulfolobus solfataricus
GAMLYLIFYDITDDNLRNRVAEFLKKKGLDRIQYSVFMGDLNSSRLKDVEAGLKIIGNRKKLQEDERFFILIVPITENQFRERIVIGYSGSEREEKSNVVW

SEQ ID NO: 1150 - protein, 101 aa, Sulfolobus solfataricus
MAMLYLIFYDITDDNLRNRVAEFLKKKGLDRIQYSVFMGDLNSSRLKDVEAGLKIIGNRKKLQEDERFFILIVPITENQFRERIVIGYSGSEREEKSNVVW

SEQ ID NO: 1151 - protein, 91 aa, Sulfolobus solfataricus
FQGMKLLVVYDVSDDSKRNKLANNLKKLGLERIQRSAFEGDMDSQRMKDLVRVVKLIVDTNTDIVHIIPLGIRDWERRIVIGREGLEEWLV

SEQ ID NO: 1152 - protein, 103 aa, Escherichia coli
MSMLVVVTENVPPRLRGRLAIWLLEVRAGVYVGDVSAKIREMIWEQIAGLAEEGNVVMAWATNTETGFEFQTFGLNRRTPVDLDGLRLVSFLPVGSSENLYFQ

SEQ ID NO: 1153 - protein, 305 aa, Escherichia coli
MTWLPLNPIPLKDRVSMIFLQYGQIDVIDGAFVLIDKTGIRTHIPVGSVACIMLEPGTRVSHAAVRLAAQVGTLLVWVGEAGVRVYASGQPGGARSDKLLYQAKLALDEDLRLKVVRKMFELRFGEPAPARRSVEQLRGIEGSRVRATYALLAKQYGVTWNGRRYDPKDWEKGDTINQCISAATSCLYGVTEAAILAAGYAPAIGFVHTGKPLSFVYDIADIIKFDTVVPKAFEIARRNPGEPDREVRLACRDIFRSSKTLAKLIPLIEDVLAAGEIQPPAPPEDAQPVAIPLPVSLGDAGHRSS

SEQ ID NO: 1154 - protein, 964 aa, Thermobifida fusca
MGSSHHHHHHSSGLVPRGSHMPEHDSTDDKHGIPPLDLRFWAKERGLRGKTYPLVCHSLDAAAAALVLWNEYLSPGLRDTIASSMETDEEHAGHCIAFWAGLHDIGKLTREFQQQIAIDLSAYPGEELSGEQRSHAAATGKWLPFALPSLGYPNGGLVTGLVAQMLGGHHGTFHPHPSFQSRNPLAEFGFSSPHWEKQRHALLHAVFDATGRPTPPDMLDGPTASVVCGLVILADWLVSQEDFLLERLTSLPADGSASALRAHFETSLRRIPSLLDAAGLRPITVPPATFTESFPHLSKPNGLQASLAKHLPCLCTGPGLVLITAPMGEGKTEAAYHVADLLGKATGRPGRFLALPTMATADQMHTRLKEYARYRVENTDLPRSSTLALLHSMAWLNPDYAPADLPGVSKVLSNLGHRDPFAATDWLMGRKRGLLAPWAVGTIDQALMAVLRAKHNALRLFGLAGKVVVVDEAHAVDPYMQVLLEQLLRWLGTLDVPVVLLSATLHHSIANSLVKAYLEGARGRRWNRSEPQPVSEVSYPGWLHVDARIGKVTRSSDVDPLPIATTPRKPLEVRLVDVPVKEGALNRSTVLAKELTPLVKQGGCAAIICTTVAEAQGVYDLLSQWFATLGEDAPDLYLLHSRFPNRQRTEITATIVDLFGKEGAQSGRRPTRGAVLVATQVVEQSLDLDVDLMISDLAPVSLLLQRAGRCWRHEHLGIINRPQWAKQPELVVLTPEQNGDADRAPWFPRSWTSVYPLALLQRTYTLLRRRNGAPVQIPEDVQQLVDDVYDDDSLAEDLEADMERMGEELAQRGLARNAVIPDPDDAEDNLNGLTEFSFDVDEHVLATRFGAGSVRVLCYYVDTAGNRWLDPECTVEFPEQGTGREGRFTMADCRDLVARTIPVRMGPWASQLTEDNHPPEAWRESFYLRDLVLIPQRVTDEGAVLPTETGGREWLLDPCKGLIF

SEQ ID NO: 1155 - protein, 964 aa, Thermobifida fusca; identical to SEQ ID NO: 1154

SEQ ID NO: 1156 - protein, 964 aa, Thermobifida fusca; identical to SEQ ID NO: 1154

SEQ ID NO: 1157 - protein, 964 aa, Thermobifida fusca; identical to SEQ ID NO: 1154

SEQ ID NO: 1158 - protein, 244 aa, Methanocaldococcus jannaschii
MILGEIMEVLAFKNQSLIDHVNDMVKYWERIKYRYLKTIKRALEALNIKLDIEKVDEFMKILIKLHDIGKASKIYQRAIINDQEKLMGFRHELVSAYYTYHILLKKFGDKNLAFIGALTVMLHHEPIIMGQIRNLKKKELTAEVVLDKLKKFDGMIEDFEDLIKKLIGYSIGDIIKNDSNKDDIIRFVIEMSVRARHTPNSEKLRFIVGTLLLPLVMCDYKGAESREGKAPKFAEVLEVESYVI

SEQ ID NO: 1159 - protein, 265 aa, Thermus thermophilus
MSHHHHHHSMAALALWAKSGNPFHPLLAHMLDTAAVALAVLRMEPPRTRALYAEDWGLPEEGALAWAAALVGLHDLGKASPVFQAGWEEGKERVQRAGLPFGELLDWVAHGVFTELFLRRLLKEKGLPERAANDLAAALGAHHGFPANAEEKSRARRHLRTEDPLWKEARRWLLEEVFRRLGAPLPPSQGNGEARPEAVLRVMALASFADWVASDPSLFPYGRDPRRGDYLKEALRLAQEALNRLGWPAFAKAQRREFGELFPYI

SEQ ID NO: 1160 - protein, 206 aa, Sulfolobus solfataricus
YFQGMITEFLLKKKLEEHLSHVKEENTIYVTDLVRCPRRVRYESEYKELAISQVYAPSAILGDILHLGLESVLKGNFNAETEVETLREINVGGKVYKIKGRADAIIRNDNGKSIVIEIKTSRSDKGLPLIHHKMQLQIYLWLFSAEKGILVYITPDRIAEYEINEPLDEATIVRLAEDTIMLQNSPRFNWECKYCIFSVICPAKLT

SEQ ID NO: 1161 - protein, 235 aa, Mannheimia succiniciproducens
MSLANRIRLHIWGDYACFTRPEMKVERVSYDVITPSAARGILSAIHWKPAINWVIDKIYVLKPIRFESVRRNELGAKISESKVSGAMKRKSVADLYTVIEDDRQQRAATVLKDVAYVIEAHAVMTSKAGVDENTTKHIEMFKRRALKGQCFQQPCMGVREFPAHFALIDDNDPLPLSQLSESEFNRDLGWMLHDIDFEHGNTPHFFRAELKNGVIDVPPFYAEEVKREGHHHHHH

SEQ ID NO: 1162 - protein, 226 aa, Xanthomonas oryzae pv. Oryzae
GHMSYGVRLHVWGERALFTRPEMKVERVSYDIITPSAARGILEAIHWKPAIRWVVDSIQVLKPICFESIRRNEVGSKLSAASISKAIKAGRTDELVKYVEEDRQQRAATVLREVGYIIAAHFEMTDKAGPDDNVGKHLDIFNRRARRGQCFQAPCLGTREFPASFALLGDDDTPPASDPALSGERDLGWMLHDIDFADGMTPRFFRARMVDGLVAVPPPQDGGVRA

SEQ ID NO: 1163 - protein, 244 aa, Streptococcus pyogenes serotype
GHMYRSRDFYVRVSGQRALFTNPATKGGSERSSYSVPTRQALNGIVDAIYYKPTFTNIVTEVKVINQIQTELQGVRALLHDYSADLSYVSYLSDVVYLIKFHFVWNEDRKDLNSDRLPAKHEAIMERSIRKGGRRDVFLGTRECLGLVDDISQEEYETTVSYYNGVNIDLGIMFHSFAYPKDKKTPLKSYFTKTVMKNGVITFKAQSECDIVNTLSSYAFKAPEEIKSVNDECMEYDAMEKGEN

SEQ ID NO: 1164 - protein, 191 aa, pseudomonas aeruginosa
GSFTMDHYLDIRLRPDPEFPPAQLMCVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARTLDLPFVTLRSQSTGQHFRLFIRHGPLQVTAEEGGFTCYGLSKGGFVPWF

SEQ ID NO: 1165 - protein, 211 aa, Thermus thermophilus
MWLTKLVLNPASRAARRDLANPYEMHRTLSKAVSRALEEGRERLLWRLEPARGLEPPVVLVQTLTEPDWSVLDEGYAQVFPPKPFHPALKPGQRLRFRLRANPAKRLAATGKRVALKTPAEKVAWLERRLEEGGFRLLEGERGPWVQILQDTFLEVRRKKDGEEAGKLLQVQAVLFEGRLEVVDPERALATLRRGVGPGKALGLGLLSVAP

SEQ ID NO: 1166 - protein, 273 aa, Pyrococcus furiosus
MAHHHHHHGSRFLIRLVPEDKDRAFKVPYNHQYYLQGLIYNAIKSSNPKLATYLHEVKGPKLFTYSLFMAEKREHPKGLPYFLGYKKGFFYFSTCVPEIAEALVNGLLMNPEVRLWDERFYLHEIKVLREPKKFNGSTFVTLSPIAVTVVRKGKSYDVPPMEKEFYSIIKDDLQDKYVMAYGDKPPSEFEMEVLIAKPKRFRIKPGIYQTAWHLVFRAYGNDDLLKVGYEVGFGEKNSLGFGMVKVEGNKTTKEAEEQEKITFNSREELKTGV

SEQ ID NO: 1167 - protein, 328 aa, Sulfolobus solfataricus
MHHHHHHMISGSVRFLVNLESLNGVESIGNLTKHRTAPVVLKTSTGYLVRYVPVISGEALAHAYQASLVDIAKKEGLPVGSLSSQYEFIKFSTDEALKIEGIKEPKDYNDARRFEVEVMLKDVIADVGGFMYAGGAPVRRTSRIKLGYMIPALRGDEIPAQLEAQFHVRFSNKPVSGSQAIFNVEVSSALYTFSFELDEDLIAVPSTFGEKVKGEEELERQKAKRVKSAIKALYSLLSGNFGGKRSRFLPSMKLMSLVVTKTDFPFMPEPAHDDDYIKTTIMRLGKAKGVLNGNLAKAYVINNEGIEVGEGVTVLSTVEDLVVKLEEE

SEQ ID NO: 1168 - protein, 351 aa, Methanopyrus kandleri
MVGIGGTITLVGEIRLRTGTRIGTSEEEIEIGGLDNPVIRDPVSGYPYVPGSSLKGRARALFELAWMKSREIEPDVFFGAHHNERHECGFVRREVYEEAKEYLREDPPWLENGTCPVCRIFGSAGDGIGFSDPGRLEDERRGLGYDPYGRYRDPNDAQELSGVVDVKKEARVAFRDAHPTTYTVNDVFERAGEPTEVKHENAINRVSGEANPRSMERVPKGSRFGLEVVYRVEDGEELESDLKYLMSSLKLVEDQGIGHSTSRGYGRVEFRIAALCARSTGWYLDPGAGEGFPEEEDKDEAADEVTYLSDLEAERYEIVIRARDLEDRAYLRPEEWVERLDEVVGELPWGR

SEQ ID NO: 1169 - protein, 506 aa, thermus thermophilus
GAGSMGSLEKFNLIDEPWIPVLKGGRVVEVGIGEALLRAHEFARIETPSPLEEAVLHRLLLAVLHRALSGPRCPEDVLDWWRKGGFPQDPIRDYLNRFRDRFFLFHPEAPFLQVADLPEENPLPWSKLLPELASGNNPTLFDHTTEENLPKATYAQAARALLVHQAFAPGGLLRRYGVGSAKDAPVARPALFLPTGQNLLETLLLNLVPYTPEDDAPIWEVPPLRLGDLEGARTKWPLTGRTRVYTWPARGVRLLDEGDGVRFMGYGPGVEPLEATHRDPMVAQRLDAKGNLLVLRLSEERSFWRDFSAMLPRQGGKVAATLEHAENLQGELEDEGLEGRITLRVLGQVSDQAKVLDIRREVYPLPSGLLTPKAEENLEKALKMAEELGQGLKHLAQEVAKAVVGERDRGHGRSPYLEELTKLANSLPLERLYWHALDGAFPRFFARVEEEASLDLWREALRGAALEAWKATRRFLGTGARHLKALAQGEQEFGRLLGELGEEVRT

SEQ ID NO: 1170 - protein, 1101 aa, Actinomyces naeslundii
MWYASLMSAHHLRVGIDVGTHSVGLATLRVDDHGTPIELLSALSHIHDSGVGKEGKKDHDTRKKLSGIARRARRLLHHRRTQLQQLDEVLRDLGFPIPTPGEFLDLNEQTDPYRVWRVRARLVEEKLPEELRGPAISMAVRHIARHRGWRNPYSKVESLLSPAEESPFMKALRERILATTGEVLDDGITPGQAMAQVALTHNISMRGPEGILGKLHQSDNANEIRKICARQGVSPDVCKQLLRAVFKADSPRGSAVSRVAPDPLPGQGSFRRAPKCDPEFQRFRIISIVANLRISETKGENRPLTADERRHVVTFLTEDSQADLTWVDVAEKLGVHRRDLRGTAVHTDDGERSAARPPIDATDRIMRQTKISSLKTWWEEADSEQRGAMIRYLYEDPTDSECAEIIAELPEEDQAKLDSLHLPAGRAAYSRESLTALSDHMLATTDDLHEARKRLFGVDDSWAPPAEAINAPVGNPSVDRTLKIVGRYLSAVESMWGTPEVIHVEHVRDGFTSERMADERDKANRRRYNDNQEAMKKIQRDYGKEGYISRGDIVRLDALELQGCACLYCGTTIGYHTCQLDHIVPQAGPGSNNRRGNLVAVCERCNRSKSNTPFAVWAQKCGIPHVGVKEAIGRVRGWRKQTPNTSSEDLTRLKKEVIARLRRTQEDPEIDERSMESVAWMANELHHRIAAAYPETTVMVYRGSITAAARKAAGIDSRINLIGEKGRKDRIDRRHHAVDASVVALMEASVAKTLAERSSLRGEQRLTGKEQTWKQYTGSTVGAREHFEMWRGHMLHLTELFNERLAEDKVYVTQNIRLRLSDGNAHTVNPSKLVSHRLGDGLTVQQIDRACTPALWCALTREKDFDEKNGLPAREDRAIRVHGHEIKSSDYIQVFSKRKKTDSDRDETPFGAIAVRGGFVEIGPSIHHARIYRVEGKKPVYAMLRVFTHDLLSQRHGDLFSAVIPPQSISMRCAEPKLRKAITTGNATYLGWVVVGDELEINVDSFTKYAIGRFLEDFPNTTRWRICGYDTNSKLTLKPIVLAAEGLENPSSAVNEIVELKGWRVAINVLTKVHPTVVRRDALGRPRYSSRSNLPTSWTIE

SEQ ID NO: 1171 - protein, 1372 aa, Streptococcus pyogenes
GSGHMDKKYSIGLAIGTNSVGWAVITDEYKVPSKKFKVLGNTDRHSIKKNLIGALLFDSGETAEATRLKRTARRRYTRRKNRILYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIEEFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1172 - protein, 1372 aa, Streptococcus pyogenes
GAASMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1173 - protein, 693 aa, Pyrococcus furiosus
MRGSHHHHHHGMASMTGGQQMGRDLYDDDDKDHPFTSVKDPTLLRIKIVPVQPFIANSRKQLDLWASSHLLSMLMYKALEVIVDKFGPEHVIYPSLRDQPFFLKFYLGENIGDEILVANLPNKALAIVSGKEAEKIEEEIKKRIRDFLLQLYREAVDWAVENGVVKVDRSEKDSMLKEAYLKIVREYFTVSITWVSLSEKEDIYQVTENAGLSDEDVKKWLKFAEKKENSRVLERIAIYPLLVKILDSLGERKVTEERFEKSEQLKGWKCHVCGENLAIFGDMYDHDNLKSLWLDEEPLCPMCLIKRYYPVWIRSKTGQKIRFESVVDVALLYKNWRKIFDEKYGKDLVSKAREVSEDFVKDNMLVDSDLYYSSTWESGLSKKLKNKKEIDEEKVKEVVDFLNAAYKEIGNPPKYYAILVMDGDDMGKVISGEVLGEISTRIHPNIRDYVEIPEAKYYSTPQVHVAISQALANFSIREVRSVVKDEGLLIYAGGDDVLAILPVDKALEVAYKIRKEFGKSFENGSLLPGWKLSAGILIVHYKHPLYDALEKARDLLNNKAKNVPGKDTLAIGLLKRSGSYYISLVGWELIRVFYNSELRKKLLEEKGGVGKRFIYHVLREVDTWPKVGIDEMLKFEVIRHIRGRNKEETKELREKIYGEIKDLLEHVRGNNEVEKVRGLFTFLKIITDAEVFP

SEQ ID NO: 1174 - protein, 686 aa, Pyrococcus furiosus
MADTRFPDHAIWTHLDLTSALSVKDPTLLRIKIVPVQPFIANSRKQLDLWASSHLLSMLMYKALEVIVDKFGPEHVIYPSLRDQPFFLKFYLGENIGDEILVANLPNKALAIVSGKEAEKIEEEIKKRIRDFLLQLYREAVDWAVENGVVKVDRSEKDSMLKEAYLKIVREYFTVSITWVSLSEKEDIYQVTENAGLSDEDVKKWLKFAEKKENSRVLERIAIYPLLVKILDSLGERKVTEERFEKSEQLKGWKCHVCGENLAIFGDMYDHDNLKSLWLDEEPLCPMCLIKRYYPVWIRSKTGQKIRFESVVDVALLYKNWRKIFDEKYGKDLVSKAREVSEDFVKDNMLVDSDLYYSSTWESGLSKKLKNKKEIDEEKVKEVVDFLNAAYKEIGNPPKYYAILVMDGDDMGKVISGEVLGEISTRIHPNIRDYVEIPEAKYYSTPQVHVAISQALANFSIREVRSVVKDEGLLIYAGGDDVLAILPVDKALEVAYKIRKEFGKSFENGSLLPGWKLSAGILIVHYKHPLYDALEKARDLLNNKAKNVPGKDTLAIGLLKRSGSYYISLVGWELIRVFYNSELRKKLLEEKGGVGKRFIYHVLREVDTWPKVGIDEMLKFEVIRHIRGRNKEETKELREKIYGEIKDLLEHVRGNNEVEKVRGLFTFLKIITDAEVFPLEHHHHHH

SEQ ID NO: 1175 - protein, 169 aa, Thermus thermophilus
MSPGERFLDWLKRLQGQKAWTAARAAFRRSLAFPPGAYPRAMPYVEPFLAKGDWRQEEREAHYLVAALYALKDGDHQVGRTLARALWEKAQGSASVEKRFLALLEADRDQIAFRLRQAVALVEGGIDFARLLDDLLRWFSPERHVQARWAREYYGAGASEEEKKKEVEA

SEQ ID NO: 1176 - protein, 114 aa, Thermus thermophilus
MRTRSQVWAQKAYEKVREAAKGEGRGEYRDMALKLPVLVRQAGLSQALAFVDSRGKEAHKALGNDLAQVLGYRDLRELAEAAREAELLQYLRLTREVLAAAEWFKRFAQALIEE

SEQ ID NO: 1177 - protein, 155 aa, Archaeoglobus fulgidus
MDIREIEQERASFAFKVVSDIKDKYSQNKKVQGKYSSYAEKAPTIILNNGLGATLAFFLSKLEKPIDDVDYKSINPESFGNAENIAYAFLYKHLSTWLAEGNGKDSAFSGLTNGEDPLKYIMEKTAIDVAISTEEALSILNWIKKFAKAMLEEEL

SEQ ID NO: 1178 - protein, 1302 aa, Francisella tularensis
SNASIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFQDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMANSKTGTELDYLISPVADVNGNFFDSRQAPKNMPQDADANGAYHIGLKGLMLLGRIKNNQEGKKLNLVIKNEEYFEFVQNRNN

SEQ ID NO: 1179 - protein, 1137 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDILEHHHHHH

SEQ ID NO: 1180 - protein, 820 aa, Unknown
  Deltaproteobacteria bacterium
MEKRINKIRKKLSADNATKPVSRSGPMKTLLVRVMTDDLKKRLEKRRKKPEVMPQVISNNAANNLRMLLDDYTKMKEAILQVYWQEFKDDHVGLMCKFAQPASGKFGQRALDFYSIHVTKESTHPVKPLAQIAGNRYASGPVGKALSDACMGTIASFLSKYQDIIIEHQKVVKGNQKRLESLRELAGKENLEYPSVTLPPQPHTKEGVDAYNEVIARVRMWVNLNLWQKLKLSRDDAKPLLRLKGFPSFPVVERRENEVDWWNTINEVKKLIDAKRDMGRVFWSGVTAEKRNTILEGYNYLPNENDHKKREGSLENPKKPAKRQFGDLLLYLEKKYAGDWGKVFDEAWERIDKKIAGLTSHIEREEARNAEDAQSKAVLTDWLRAKASFVLERLKEMDEKEFYACEIQLQKWYGDLRGNPFAVEAENRVVDISGFSIGSDGHSIQYRNLLAWKYLENGKREFYLLMNYGKKGRIRFTDGTDIKKSGKWQGLLYGGGKAKVIDLTFDPDDEQLIILPLAFGTRQGREFIWNDLLSLETGLIKLANGRVIEKTIYNKKIGRDEPALFVALTFERREVVDPSNIKPVNLIGVARGENIPAVIALTDPEGCPLPEFKDSSGGPTDILRIGEGYKEKQRAIQAAKEVEQRRAGGYSRKFASKSRNLADDMVRNSARDLFYHAVTHDAVLVFANLSRGFGRQGKRTFMTERQYTKMEDWLTAKLAYEGLTSKTYLSKTLAQYTSKTCSNCGFRFSHRPVQEQFVCLDCGHEVHAAEQAALNIARSWLFLNSNSTEFKSYKSGKQPFVGAWQAFYKRRLKEVWKPNA

SEQ ID NO: 1181 - protein, 1160 aa, Leptotrichia buccalis
SMKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIANYIAAFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 1182 - protein, 1440 aa, Unknown
  Lachnospiraceae bacterium
SNAMKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 1183 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 1184 - protein, 1232 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIANKFAHNQLPKKEFWDYCQEKYGKIEKEKTYAEYFAEVFKKEKEALIKLEHHHHHH

SEQ ID NO: 1185 - DNA, 35 bp, Artificial
  Synthetic
actgtttcuccattgtttttaaaccatgtcaattg

SEQ ID NO: 1186 - DNA, 34 bp, Artificial
  Synthetic
actgtttcuccattggtcgcatcacagcaaatag

SEQ ID NO: 1187 - DNA, 32 bp, Artificial
  Synthetic
actgtttcuccatccgtgttccccgcgtgtgc

SEQ ID NO: 1188 - DNA, 35 bp, Artificial; identical to SEQ ID NO: 1185
  Synthetic

SEQ ID NO: 1189 - DNA, 35 bp, Artificial
  Synthetic
acggccaguataaccaataagccgctgataatccc

SEQ ID NO: 1190 - DNA, 28 bp, Artificial
  Synthetic
acccaugagcaccatcatcgaccaggac

SEQ ID NO: 1191 - DNA, 33 bp, Artificial
  Synthetic
atggguatgtatatctccttcttaaagttaaac

SEQ ID NO: 1192 - DNA, 23 bp, Artificial
  Synthetic
accgccguggcgttagtcgattt

SEQ ID NO: 1193 - DNA, 22 bp, Artificial
  Synthetic
acggcggucaacctcatggacg

SEQ ID NO: 1194 - DNA, 38 bp, Artificial
  Synthetic
gtgttccccacgggtgtggggatgaaccactggccgtt

SEQ ID NO: 1195 - DNA, 41 bp, Artificial
  Synthetic
ggttcatccccacacccgtggggaacacatggagaaacagt

SEQ ID NO: 1196 - DNA, 239 bp, Artificial
  Synthetic
actgtttctccatttttttaaaatggtgaaagtctaacgactatttaaatttctactatttgtagatacgaattcaaggtattggcgaaaatcttttctagcgacacaaacacagcttgccaataatacttgtaaggggattttgtgcatttttaactccaaatagggtgcaaggcgtggcggtagctactcctgccttgccgtcggtgaccattgtcctaatcgtatcaactggccgt

SEQ ID NO: 1197 - DNA, 79 bp, Artificial
  Synthetic
actgtttctccattgtttttaaaccatgtcaattgtctaacaactttttaaatttctactgtttgtagatactggccgt

SEQ ID NO: 1198 - DNA, 209 bp, Artificial
  Synthetic
actgtttctccattggtcgcatcacagcaaatagcagtctaacgaccttttaaatttctactgtttgtagatttcctgttggggattatcagcggcttattggttatttgcgctttgacagattttgtgtgaaatatcctgacagcaaggcggattttaactgctttgagccgtcggtgaccattgtcctaatcgtatcaactggccgt

SEQ ID NO: 1199 - DNA, 183 bp, Artificial
  Synthetic
actgtttctccatccgtgttccccgcgtgtgcggggatgaaccgtgagagtatccgtgacgtcctgcacccttctgatgcgagtcatcaaagcccatgcccactggcgttggcgcgcctgacctccttgttctccggctcgccggcgtcggtgaccattgtcctaatcgtatcaactggccgt

SEQ ID NO: 1200 - DNA, 374 bp, Artificial
  Synthetic
tacccatgagcaccatcatcgaccaggacggcgaggaaatcgactaacgccacggcggtcaacctcatggacgatgagatccgcgaggaactccacgccgagatggcactctgcaccgatcagcagttcttcgacgcctacatcgaaaggcactacgccaagtacggcgaggatttcaccatctgacaagcacggcggtcgcgccccgcgagggggcgcgtggatcgaaacacgaccatcacggtcccacccgggccgacaccgatccaagacgggagcatccgacatggggcatcgtaacggcaaacgcctacgcctcataccatcacacctgcccgtcggtgaccattgtcctaatcgtatcaactggccgt

SEQ ID NO: 1201 - DNA, 29 bp, Artificial
  Synthetic
gtgttccccgcgtgtgcggggatgaaccg

SEQ ID NO: 1202 - DNA, 28 bp, Artificial
  Synthetic
gtgttccccacgggtgtggggatgaacc

SEQ ID NO: 1203 - DNA, 34 bp, Artificial
  Synthetic
gtcgcgccccgcgagggggcgcgtggatcgaaac

SEQ ID NO: 1204 - DNA, 36 bp, Artificial
  Synthetic
gtctaacgactatttaaatttctactatttgtagat

SEQ ID NO: 1205 - DNA, 36 bp, Artificial
  Synthetic
gtctaacaactttttaaatttctactgtttgtagat

SEQ ID NO: 1206 - DNA, 36 bp, Artificial; identical to SEQ ID NO: 18
  Synthetic

SEQ ID NO: 1207 - DNA, 27 bp, Artificial
  Synthetic
cctcactgccgtataggcagcccagaa

SEQ ID NO: 1208 - DNA, 31 bp, Artificial
  Synthetic
gttcactgccggataggcagccaaggaaatc

SEQ ID NO: 1209 - DNA, 36 bp, Artificial; identical to SEQ ID NO: 18
  Synthetic

SEQ ID NO: 1210 - DNA, 36 bp, Artificial; identical to SEQ ID NO: 1205
  Synthetic

SEQ ID NO: 1211 - DNA, 28 bp, Artificial
  Synthetic
gttcactgccgtataggcagctaagaaa

SEQ ID NO: 1212 - DNA, 36 bp, Artificial; identical to SEQ ID NO: 18
  Synthetic

SEQ ID NO: 1213 - DNA, 31 bp, Artificial; identical to SEQ ID NO: 1208
  Synthetic

(19) United States
(12) Patent Application Publication
(10) Pub. No.: US 2024/0247255 A1
(43) Pub. Date: Jul. 25, 2024

(54) METHODS FOR MODULATING CAS-EFFECTOR ACTIVITY

(71) Applicant: UNIVERSITY OF COPENHAGEN, Copenhagen (DK)

(72) Inventors: Rafael PINILLA-REDONDO, Frederiksberg (DK); Sarah Camara WILPERT, Copenhagen S. (DK); Jakob RUSSEL, Lejre (DK); Søren J. Sørensen, Holte (DK)

(21) Appl. No.: 18/005,570

(22) PCT Filed: Jul. 12, 2021
(86) PCT No.: PCT/EP2021/069286

§ 371 Date: Jan. 13, 2023

(30) Foreign Application Priority Data
Jul. 13, 2020 (EP) 20185475.9

(51) Int. Cl. C12N 15/11; A61K 48/00; C12N 9/22

(52) U.S. Cl. CPC C12N 15/11; A61K 48/005; C12N 9/22; C12N 2310/12; C12N 2310/20

(57) ABSTRACT

The present invention relates to methods of modulating an activity of a Cas-effector on a target polynucleotide comprising contacting the Cas-effector with an inhibitor component, wherein the inhibitor component comprises an anti-CRISPR ribonucleotide sequence (acrRNA) capable of inhibiting the Cas-effector from (i) associating with a target nucleotide sequence; and/or (ii) associating with a CRISPR guide RNA, and thereby inhibiting the Cas-effector from forming an active RNA-guided Cas-effector complex.

### FIELD OF THE INVENTION

The present invention relates to methods for modulating an activity of a Cas-effector on a target nucleotide using newly discovered inhibitor components comprising an anti-CRISPR ribonucleotide sequence (acrRNA). The invention also encompasses acrRNA's and compositions comprising such acrRNA's as well applications of such acrRNA's for therapy and/or diagnostics as well as for research and development purposes such as gene editing and the like.

### BACKGROUND OF THE INVENTION

Protein based technologies for modulation and control of CRISPR-Cas systems have emerged over the recent years, such as described in WO2018/197520 disclosing anti-CRISPR polypeptides modulating the activity of a Cas endonuclease or described in Marino, N. D. et al. (2020) ‘Anti-CRISPR protein applications: natural brakes for CRISPRCas technologies’, Nature Methods. Another recent patent application, WO2018/009822, discloses inhibiting CRISPR genome editing systems using chemically modified complementary guide RNA. Further, Meeske & Marraffini, Molecular Cell. 71, 791-801, Sep. 6, 2018, discloses that in type VI-A CRISPR-Cas system in its natural host, Listeria seeligeri, a guide RNA modified by adding a tag extending the complementarity to the target sequence of the flanking sequence between the protospacer and the repeat sequence of the guide RNA prevents the VI-A nuclease Cas13 from cleaving the target.

Bin Li et al; Cell Reports; vol. 25; no. 12; 2018; pages 3262 to 3272, relates to chemical modification of oligonucleotides complementary to stretches of a crRNA, thereby allegedly hindering crRNA-target DNA binding. The strand used is complementary to the crRNA (so opposite to the Single Repeat Units or SRUs). Here is also appears that random sequence of chemically modified phosphorothioate-DNA interferes with CRISPR targeting, as such modified DNA presumably have a high unpecific affinity to proteins which in turn would be toxic and mess up cellular processes that involve DNA-binding proteins.

C. Barkau et al.; Nucleic Acid Therapeutics; vol. 29; no. 3; 2019; pages 136-14,7 relates to (chemically) modified 2′-O-methyl-oligonucleotides binding to CRISPR guide RNA or repeat sequences or or DNA oligonucleotides binding to PAM with the aim at inhibiting gene editing in human cells. None of these are however RNA sequences which can be expressed in vivo in a cell and moreover they are only useful for some CRISPR systems (type II and some type V systems) employing tracrRNA. Indeed these oligonucleotides are modified chemically, and some residues have to be added to make inhibition robust.

Shao-Ru Wang et al. 2020; Nature Communications; vol. 11; no. 1; 2020 relates to chemically masking gRNA through covalent attachment of AMR groups. The masking can be reversed via a redox reaction in vitro, leading to chemical activation of the gRNAs, while the masking cannot be done in vivo by the cells.

Accordingly, there remains a tremendous need for providing further technology for modulation and control of CRISPR-Cas systems, in particular modulation and control that can be exercised in vivo in the cell through expression of CRISPR-Cas modulators.

### SUMMARY OF THE INVENTION

New ribonucleotide structures, notably (poly)ribonucleotides, have been identified, which surprisingly are capable of inhibiting or even preventing activities of Cas-effectors on target DNA. Accordingly, in a first aspect the invention provides a method for modulating an activity of a Cas-effector on a target polynucleotide comprising contacting the Cas-effector with an inhibitor component, wherein the inhibitor component comprises an anti-CRISPR ribonucleotide sequence (acrRNA) capable of inhibiting the Cas-effector from (i) associating with a target nucleotide sequence; and/or (ii) associating with a CRISPR guide RNA, and thereby inhibiting the Cas-effector from forming an active RNA-guided Cas-effector complex, wherein the arcRNA lacks a sequence (spacer) recognizing the target nucleotide sequence.

In a second aspect the invention provides acrRNAs capable of inhibiting a Cas-effector from (i) associating with a target nucleotide sequence; and/or (ii) associating with a CRISPR guide RNA, and thereby inhibiting the Cas-effector from forming an active RNA-guided Cas-effector complex, wherein the arcRNA lacks a spacer sequence of the guide RNA recognizing the target nucleotide sequence.

In a further aspect the invention provides genetically modified host cells comprising a gene encoding the acrRNA of the invention, operably linked to a controllable or constitutive regulatory expression element.

In a further aspect the invention provides compositions comprising the acrRNA of the invention.

In a further aspect the invention provides the use of acrRNA of the invention as a medicament for treating a disease.

### DESCRIPTION OF FIGURES

FIG. 1 shows the results of a phage spotting assays where lanes of bacterial lawns are spotted with phage serial dilutions

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

### Definitions

Any EC numbers used herein refers to Enzyme Nomenclature 1992 from NC-IUBMB, Academic Press, San Diego, California, including 30 supplements 1-5 published in Eur. J. Bio-chem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. http://enzyme.expasy.org/.

The term “CRISPR RNA”, “crRNA”, or “guide RNA” are used interchangeably herein and refer to a polynucleotide sequence that can form a complex with a Cas protein or multi-Cas-protein complex and guide the ribonucleoprotein complex to recognize, and potentially bind to, and optionally cleave, a target nucleotide sequence site. The guide polynucleotide sequence may be an RNA sequence, a DNA sequence, or a combination thereof, optionally including modified nucleotide bases.

The term “trans-activating CRISPR RNA” or “tracrRNA” as used herein refers to an RNA species that interacts with the crRNA to form the crRNA-tracrRNA chimeric guide employed by some CRISPR-Cas systems (e.g. all type II subtypes and some type V subtypes). The tracrRNA serves an important scaffold function for the recognition and coupling by Cas proteins. The tracrRNA-crRNA interaction is essential for pre-crRNA processing, target recognition and cleavage, as well as transcriptional autoregulation of expression in native type II systems. The tracrRNA an anti-repeat, named as such because it forms an imperfect hybrid (partially complementary) with the repeat in the crRNA repeat region.

The term “crRNA-tracrRNA fusion” as used herein refers to the fusion of two RNA molecules comprising a crRNA fused to a tracrRNA. The so-called single guide (sgRNA) or crRNA-tracrRNA fusion may comprise a complete crRNA and tracrRNA, or fragments thereof, that form a complex with a Cas effector protein (e.g. Cas9 or modified versions and homolog variants), wherein the resultant RNA-protein complex is guided by the crRNA portion to recognize a complementary, or partially complementary (Watson-Crick base-pairing), target site to the spacer, allowing the complex to optionally bind to, and potentially cleave or nick (double or single stranded DNA breaks, respectively) the target nucleic acid.

“CRISPR-Cas system” refers to Clustered regularly interspaced short palindromic repeats and their CRISPR-associated (Cas) proteins. These systems comprise a plurality of diverse RNA guided prokaryotic adaptive immune systems employed by these organisms to defend against foreign parasitic nucleic acids. CRISPR-Cas systems include type I to VI types, each of which includes multiple subtypes and variants. CRISPR-Cas systems typically comprise a CRISPR array (updatable memory bank of the immune system that includes sequences of former genetic intruders) and a cluster of Cas protein involved in different stages of immunity. It is clear to a person in the art field that CRISPR and Cas loci can be functionally linked despite not being co-localized within a genome and that diverse CRISPR-C₅ systems may overlap in Cas protein homolog content. A more extensive structural, functional, evolutionary classification can be found in Makarova, K. S. et al. (2015) ‘An updated evolutionary classification of CRISPR-Cas systems’, Nature reviews. Microbiology., 13, p. 722; and Makarova, K. S. et al. (2020) ‘Evolutionary classification of CRISPR-Cas systems: a burst of class 2 and derived variants’, Nature reviews. Microbiology, 18(2), pp. 67-83.

The term “CRISPR repeat” or “repeat sequence” as used herein refers to their conventional meaning as used in the art, that is, multiple, short, direct repeat nucleotide sequences showing reduced or no sequence variation. These sequences originate from, or are homologous to, the sequences in between the spacer sequences found within a given CRISPR array. Many repeat sequences are partially/semi-palindromic, thus potentially leading to the formation or partial adoption of stable, conserved secondary structure arrangements, e.g. stem-loop folds or hairpins.

The term “palindromic repeat sequence” as used herein refers to a repeat nucleotide sequence for which at least a portion of the repeat is equal to its reverse complement. Due to the natural Watson-Crick base-pairing properties of nucleic acids, palindromic nucleotide sequences are capable of (partially) folding over themselves forming hairpin and stem-loop secondary structures.

The term “semipalindromic repeat sequence” as used herein refers to repeat sequences embedded within CRISPR-derived repeats which do not comprise perfect or full-length palindromes, meaning that, either portions or certain punctual nucleotides across the predicted palindrome, are not functionally predisposed to base-pair.

The term “cognate” as used herein refers to interacting pairs of functional entities. More specifically, that each protein or protein effector complex having RNA guided activity has a cognate crRNA and/or crRNA-tracrRNA fusion, that are required for their activity upon recognition of the targeted site.

The term “spacer” as used herein refers to sequences interspersed among the direct repeat sequences of CRISPR arrays and which, after CRISPR array transcription and processing into mature crRNAs, comprise a portion of the crRNAs that guide the Cas effector protein(s) to a complementary site (so-called the protospacer). In nature, spacer sequences within CRISPR arrays are known to derive from the genomes of viral and other invading genetic elements, thus comprising the memory-basis for the adaptive immune response against recidivist threats. Note that each crRNA contains only one repeat sequence and a variable portion of one of the adjacent repeats in the CRISPR array from which it was transcribed.

The terms “type X CRISPR-Cas system”, where X refers to either I, II, III, IV, V, or VI, as used herein refer to the different 6 types of CRISPR-Cas systems hitherto described in literature. A thorough description of their specific functional components and evolutionary relationships is detailed in Makarova et al 2015 and Makarova et al 2020.

The terms “CasX”, where X refers to numbers 1 to 14, DinG, RecD, and LS as used herein refer to the Cas protein components (and homologs thereof, or modified versions thereof) involved in the functioning of the different CRISPR-Cas systems. A thorough description of their properties, classification and evolution is described in Makarova et al 2015 and Makarova et al 2020. In some embodiments, Cas protein nucleases (e.g. Cas9, Cas12, etc.) can be defective. For instance, the Cas nuclease can perform nicks in the target DNA, rather than a double strand breakage or have been modified to have deactivated nuclease domains (catalytically dead Cas variants), thus allowing for programmable nucleic acid recognition and potentially binding, without nuclease activity. In other embodiments, Cas proteins which retain the activity to be RNA-guided to a given target site can additionally comprise additional functionalities, such as for example through the fusion of, or conjugation/linkage with, other proteins and/or functional moieties, including fluorophores or fluorescent proteins, transcription factors (activators or repressors), DNA/chromatin remodelling effectors, epigenetic modifiers (methyltransferases, acetylases, etc.), prime/base editors (cytidine/(deoxy)adenosine deaminases), affinity tags, retrons, polymerases (reverse transcriptases or error-prone DNA polymerases), among others.

The terms “heterologous” or “recombinant” or “genetically modified” and its grammatical equivalents as used herein interchangeably refers to entities “derived from a different species or cell”. For example, a heterologous or recombinant polynucleotide gene is a gene in a host cell not naturally containing that gene, i.e. the gene is from a different species or cell type than the host cell. The terms as used herein about microbial host cells refers to microbial host cells comprising and expressing heterologous or recombinant polynucleotide genes.

The term “in vivo”, as used herein refers to within a living cell, including, for example, a microorganism or a human cell or a plant cell.

The term “ex vivo”, as used herein refers to when a given experiment or procedure is conducted outside a given biological organism, cell or tissue (e.g. a bacterium, human organs, mammalian cell lines), and are thus conducted directly in a laboratory environment, with attention to minimally altering the organism or cell natural in vivo conditions.

The term “in vitro”, as used herein refers to outside a living cell, including, without limitation, for example, in a microwell plate, a tube, a flask, a beaker, a tank, a reactor and the like.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, using these deviating terms can also include a range deviation plus or minus such as plus or minus 9%, 8%, 7%, 6%, 5%, 4%, 3%, 2%, or 1% from a specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” or “recovered” as used herein about a compound, refers to any compound, which by means of human intervention, has been put in a form or environment that differs from the form or environment in which it is found in nature. Isolated compounds include but is no limited to compounds of the disclosure for which the ratio of the compounds relative to other constituents with which they are associated in nature is increased or decreased. In an important embodiment the amount of compound is increased relative to other constituents with which the compound is associated in nature. In an embodiment the compound of the disclosure may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “% identity” is used herein about the relatedness between two amino acid sequences or between two nucleotide sequences. “% identity” as used herein about amino acid sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

$$\frac{iden tical amino acid residues}{L ength of alignment - total number of gaps in alignment} \times 100$$

“% identity” as used herein about nucleotide sequences refers to the degree of identity in percent between two nucleotide sequences obtained when using the Needleman-Wunsch algorithm

(Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

$$\frac{identical deoxyribonucleotides}{L ength of alignment - total number of gaps in alignment} \times 100$$

The protein sequences of the present disclosure can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold.

Accordingly, the program calculates the identity only for these matching segments. Therefore, the identity calculated in this way is referred to as local identity.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “host cell” refers to any cell type that is susceptible to transformation, transfection, transduction, or the like with a polynucleotide construct or expression vector comprising a polynucleotide of the present disclosure. The term “host cell” encompasses any progeny of a parent cell that is not identical to the parent cell due to mutations that occur during replication. The term “polynucleotide construct” refers to a polynucleotide, either single- or double

stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “operably linked” refers to a configuration in which a control sequence is placed at an appropriate position relative to the coding polynucleotide such that the control sequence directs expression of the coding polynucleotide.

The terms “nucleotide sequence and “polynucleotide” are used herein interchangeably.

The term “comprise” and “include” as used throughout the specification and the accompanying claims as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the claimed disclosure or to imply that certain features are critical, essential, or even important to the structure or function of the claimed disclosure. Rather, these terms are merely intended to highlight alternative or additional features that can or cannot be utilized in a particular embodiment of the present disclosure.

### Methods Provided by Invention

As described, supra, the present invention evolves from the inventor's discovery of ribonucleotide sequences, which can interact with CRISPR-Cas systems both in vivo in a living cell and ex vivo and modulate the activity of the Cas effector. Accordingly, in a first aspect the in the methods provided invention comprise modulating an activity of a Cas-effector on a target polynucleotide comprising contacting the Cas-effector with an inhibitor component, wherein the inhibitor component comprises an anti-CRISPR ribonucleotide sequence (acrRNA) capable of inhibiting the Cas-effector from (i) associating with a target nucleotide sequence; and/or (ii) associating with a CRISPR guide RNA for the Cas-effector, and thereby inhibiting the Cas-effector from forming an active RNA-guided Cas-effector complex. The acrRNA may inhibit the Cas-Effector to a varying degree from weak to moderate to strong to even completely prevent the Cas-effector from (i) associating with the target nucleotide sequence; and/or (ii) associating with the CRISPR guide RNA, and thereby prevents the Cas-effector from forming an active RNA-guided Cas effector complex. The guide RNA can in particular be a CRISPR RNA (crRNA), include a trans-activating CRISPR RNA (tracrRNA); and/or be a fusion of a crRNA and a tracrRNA (crRNA-tracrRNA fusion).

In a preferred embodiment the modulating property of the acrRNA is accomplished by the acrRNA comprising a ribonucleotide sequence having a high similarity to the structural moiety of the CRISPR guide RNA, which binds to one or more components of a given Cas-effector, but where the arcRNA lacks one or more spacer sequences of the guide RNA recognizing the target nucleotide sequence. In some embodiments the acrRNA comprises a ribonucleotide sequence having at least 70% identity to a sequence of the structural moiety of the CRISPR guide RNA, which binds to one or more components of the corresponding Cas-effector, but wherein the arcRNA lacks one or more spacer sequences of the guide RNA recognizing the target nucleotide sequence. In more specific embodiments the acrRNA comprises a ribonucleotide sequence that is at least 70%, such as at least 75%, such as at least 80%, such as at least 85%, such as at least 90%, such as at least 95%, such as at least 97%, such as at least 98%, such as at least 99%, such as 100% identical to any one of SEQ ID NO: 10 through 13 all sequences separately included.

In further embodiments the acrRNA may comprise at least one repeat sequence of the structural moiety of the CRISPR guide RNA, which binds to the one or more components of the corresponding Cas-effector. Such repeat sequences can be palindromic, semi-palindromic and/or cognate repeat sequences. Moreover, such repeat sequences is selected from a type I, type III, type IV, type V, type VI CRISPR-Cas system repeat sequence. More specifically the repeat sequence has at least 70%, such as at least 75%, such as at least 80%, such as at least 85%, such as at least 90%, such as at least 95%, such as at least 97%, such as at least 98%, such as at least 99%, such as 100% identity to the repeat sequence comprised in SEQ ID NO: 14 to 929 all sequences separately included.

In still further embodiments the acrRNA may comprise one or more moieties hybridizing to the CRISPR guide RNA and thereby inhibit the CRISPR guide RNA from associating with the Cas-effector. Such hybridizing moieties may include anti-repeat ribonucleotide sequences complementary to a repeat sequence of the CRISPR guide RNA. Particularly, such an anti-repeat sequence can be at least 70%, such as at least 75%, such as at least 80%, such as at least 85%, such as at least 90%, such as at least 95%, such as at least 97%, such as at least 98%, such as at least 99%, such as 100% identical to the sequence complementary to the repeat sequence comprised in SEQ ID NO: 14 to 929 all sequences separately included.

In still further embodiments the acrRNA can modulate Cas-effectors which are selected from type I, type III, type IV, type V and/or type VI Cas-effectors. Such Cas-effectors may comprise a Cas3, Cas5, Cas6, Cas7, Cas8, Cas10, DinG, RecD, LS, Cas11, Cas9, Cas12, Cas12f, Cas13 and/or Cas14 protein complex. These protein complexes may have RNA-guided nuclease activity, they may be catalytically inactive (dCas) or have single stranded nickase function (nCas), instead of a double stranded nuclease activity. More specifically the protein complex can comprise an amino acid sequence which is at least 70% identical to SEQ ID NO: 1146 to 1184 all sequences separately included.

Where the guide RNA is a crRNA, the crRNA may be a type I, type III, type IV, type V and/or type VI CRISPR-Cas system crRNA.

Where the guide RNA includes a tracrRNA, the tracrRNA may be a type II and/or type V CRISPR-Cas system tracrRNA. More specifically the tracrRNA can have has at least 70%, such as at least 75%, such as at least 80%, such as at least 85%, such as at least 90%, such as at least 95%, such as at least 97%, such as at least 98%, such as at least 99%, such as 100% identity to the tracrRNA comprised in SEQ ID NO: 930 to 1145 all sequences separately included.

Where the guide RNA is a crRNA-tracrRNA fusion, the crRNA-tracrRNA fusion may be a type II or type V CRISPR-Cas system crRNA-tracrRNA fusion.

The methods provided for herein can be performed by contacting of the Cas-effector with the inhibitor component in vivo in a living cell or ex vivo. When performing the method in vivo in a living cell, such cell can be a eukaryotic cell, a prokaryotic cell or an archaeal cell. Particularly the cell can be eukaryotic, such as a mammalian cell, a plant cell, an insect cell, or a fungal cell. In some embodiments the mammalian cell can be an animal cell or human cell, optionally a blood or an induced pluripotent stem cell.

When performing the methods provided for herein in vivo in a living cell, may by encoded by a transgene comprised in the cell. In an embodiment the transgene can be comprised in a self-replicating genetic element. The transgene encoding the acrRNA is preferably operably linked to a native or heterologous regulatory expression element, which in some embodiments may be controllable in response to selected conditions. Such conditions can be selected from one or more of temperature, presence or absence of a molecule/ligand, activation or suppression of an endogenous gene, light, sound, cell cycle, organism phase, tissue, cell type and/or environmental stress. The regulatory expression element may also be constitutive.

In an embodiment alternative to having the cell express the acrRNA, the acrRNA can also be fed exogenously to the cell, optionally by contacting the cell with the acrRNA and/or a delivery vehicle comprising the acrRNA. Suitable delivery forms include lipofection, nucleofection, microinjection, biolistics, virosomes, liposomes, immunoliposomes, polycation or lipid-nucleic acid conjugates, naked DNA, and artificial or phage-based virions.

The methods provided for herein may also be performed ex vivo, where the Cas-effector is contacted with the inhibitor component outside a living cell. In such ex vivo methods the contacting of the Cas-effector with the inhibitor component can be performed by preparing a medium comprising an extract of the cells provided for herein comprising the Cas-effector and the acrRNA or genes encoding them and providing for cell-free transcription-translation protein synthesis in the medium. Optionally, the medium may also provide for DNA and/or RNA synthesis.

acrRNAs and Compositions Provided by the Invention

In a further aspect the invention provides acrRNA's capable of inhibiting a Cas-effector from
(i) associating with a target nucleotide sequence; and/or (ii) associating with a CRISPR guide RNA, and thereby inhibiting the Cas-effector from forming an active RNA-guided Cas-effector complex. The acrRNA may inhibit the Cas-Effector to a varying degree from weak to moderate to strong to even completely prevent the Cas-effector from (i) associating with the target nucleotide sequence; and/or
(ii) associating with the CRISPR guide RNA, and thereby prevents the Cas-effector from forming an active RNA-guided Cas effector complex. The guide RNA can in particular be a CRISPR RNA (crRNA), include a trans-activating CRISPR RNA (tracrRNA); and/or be a fusion of a crRNA and a tracrRNA (crRNA-tracrRNA fusion). In a particular embodiment the acrRNA comprises a ribonucleotide sequence having at least 70% identity to a sequence of the structural moiety of the CRISPR guide RNA, which binds to one or more components of the corresponding Cas-effector, and wherein the arcRNA lacks a spacer sequence of the guide RNA recognizing the target nucleotide sequence. Suitable acrRNA's comprises a ribonucleotide sequence that is at least 70%, such as at least 75%, such as at least 80%, such as at least 85%, such as at least 90%, such as at least 95%, such as at least 97%, such as at least 98%, such as at least 99%, such as 100% identity to any one of SEQ ID NO: 10 to 13, or 1201 to 1213 all sequences separately included. In further embodiments, the acrRNA comprises at least one repeat sequence of the structural moiety of the CRISPR guide RNA, which binds to the one or more components of the corresponding Cas-effector. Said repeat sequence may be palindromic, semi-palindromic and/or cognate and may be selected from a type I, type III, type IV, type V, type VI CRISPR-Cas system repeat sequence. Suitable repeat sequences have at least 70%, such as at least 75%, such as at least 80%, such as at least 85%, such as at least 90%, such as at least 95%, such as at least 97%, such as at least 98%, such as at least 99%, such as 100% identity to the repeat sequence comprised in SEQ ID NO: 10 to 929 all sequences separately included. In still further embodiments the acrRNA comprises a moiety hybridizing to the CRISPR guide RNA and thereby inhibits the CRISPR guide RNA from associating with the corresponding Cas-effector. The hybridizing moiety can be an anti-repeat sequence complementary to a repeat sequence of the CRISPR guide RNA. Suitable anti-repeat sequences are at least 70%, such as at least 75%, such as at least 80%, such as at least 85%, such as at least 90%, such as at least 95%, such as at least 97%, such as at least 98%, such as at least 99%, such as 100% identical to a sequence complementary to the repeat sequence comprised in SEQ ID NO: 10 to 929 all sequences separately included. The Cas-effectors modulated by the acrRNA of the invention may be selected from a type I, type III, type IV, type V and/or type VI Cas-effector. Such Cas-effectors may comprise a Cas3, Cas5, Cas6, Cas7, Cas8, Cas10, DinG, RecD, LS, Cas11, Cas9, Cas12, Cas12f, Cas13 and/or Cas14 protein complex and such protein complex may have RNA-guided nuclease activity or may be catalytically inactive (dCas). For catalytically inactive Cas-effectors one activity modulated by the acrRNA of the invention is chromatin remodelling, prime/base editing, recruitment of other effector proteins or molecules, optionally via fusion, potentially including a linker sequence connecting them. Examples of potential functional domains that may be fused to a Cas protein include, without limitation, epitope tags (e.g. histidine tags, V5 tags, FLAG tags, influenza hemagglutinin tags, Myc tags, VSV-G tags, and thioredoxin tags), reporters (e.g. horseradish peroxidase, chloramphenicol acetyltransferase, beta-galactosidase, beta-glucuronidase, luciferase, glutathione-5-transferase, green fluorescent protein, HcRed, DsRed, cyan fluorescent protein, yellow fluorescent protein, flavin mononucleotide-based fluorescent proteins and autofluorescent proteins including blue fluorescent protein) and protein domains with one or more of the following functions: methylase or demethylase activity, transcription activation or transcription repression activity, transcription release factor activity, polymerases, histone modification activity, RNA cleavage activity and nucleic acid binding activity. The modulated Cas-effector protein complex can comprise an amino acid sequence which at least 70%, such as at least 75%, such as at least 80%, such as at least 85%, such as at least 90%, such as at least 95%, such as at least 97%, such as at least 98%, such as at least 99%, such as 100% identical to SEQ ID NO: 1146 to 1184 all sequences separately included. In a particular embodiment the acrRNA of the invention inhibits the Cas-effector from forming an active RNA-guided Cas-effector complex with crRNA of type I, type III, type IV, type V and/or type VI CRISPR-Cas systems. In another particular embodiment the acrRNA of the invention inhibits the Cas-effector from forming an active RNA-guided Cas-effector complex with tracrRNA of type II and/or type V CRISPR-Cas systems, such as those tracrRNA's that have at least 70%, such as at least 75%, such as at least 80%, such as at least 85%, such as at least 90%, such as at least 95%, such as at least 97%, such as at least 98%, such as at least 99%, such as 100% identity to the tracrRNA comprised in SEQ ID NO: 930 to 1145 all sequences separately included. In another particular embodiment the acrRNA of the invention inhibits the Cas-effector from forming an active RNA-guided Cas-effector complex with a crRNA-tracrRNA fusion of type II and/or type V CRISPR-Cas systems.

The present invention also provides compositions comprising the acrRNA of the invention the delivery comprising the acrRNA. Such compositions may further include suitable carriers, excipients, agents, additives and/or adjuvants and in particular the composition is a pharmaceutical composition comprising one or more pharmaceutical grade carriers, excipients, agents, additives and/or adjuvants.

### Genes and Constructs Provided by the Invention

In a further aspect the invention provides genes encoding the acrRNA of the invention. In particular such genes comprises a nucleotide sequence which is at least 70%, such as at least 75%, such as at least 80%, such as at least 85%, such as at least 90%, such as at least 95%, such as at least 97%, such as at least 98%, such as at least 99%, such as 100% identical to the gene encoding the acrRNA comprised in anyone of SEQ ID NO: 10 to 13, or 1201 to 1213 all sequences separately included or genomic DNA thereof. The gene encoding the acrRNA, may be operably linked to one or more regulatory expression elements. Such regulatory expression elements may be controllable or constitutive. Controllable regulatory expression elements may respond to various conditions such as one or more conditions selected from: temperature, presence or absence of a molecule/ligand, activation or suppression of an endogenous gene, light, sound, cell cycle, organism phase, tissue, cell type or environmental stress.

### Vehicles and Cells Provided by the Invention

In a further aspect the invention provides delivery vehicles or cells comprising the acrRNA of the invention. In one embodiment the delivery vehicle optionally comprising a liposome, nanoparticle or a phage particle. In another embodiment the cell is a genetically modified host cell comprising the gene or the nucleotide construct of the invention providing for the expression of acrRNA.

Applications of acrRNAs Provided by the Invention

In a further aspect the invention provides applications and uses of acrRNA of the invention. Particularly the arRNA may be used as a medicament for treatment of a disease or malfunction in a living organism or for diagnosing such malfunctions.

### Sequence Listings

The present application contains a Sequence Listing prepared in Patent In version 3.5.1, which is also submitted electronically in ST25 format which is hereby incorporated by reference in its entirety.

| Sequence | Type | Description |
|---|---|---|
| SEQ ID NO: 1 | DNA/RNA | For pHerd30t backbone amplification |
| SEQ ID NO: 2 | DNA/RNA | For pHerd30t backbone amplification |
| SEQ ID NO: 3 | DNA/RNA | For sequencing of repeat/acrRNA site |
| SEQ ID NO: 4 | DNA/RNA | For sequencing of repeat/acrRNA site |
| SEQ ID NO: 5 | DNA/RNA | For amplification of acrRNAs |
| | | ordered from twist bioscience for |
| | | insertion into pHerd30T |
| SEQ ID NO: 6 | DNA/RNA | For amplification of acrRNAs |
| | | ordered from twist bioscience for |
| | | insertion into pHerd30T |
| SEQ ID NO: 7 | DNA/RNA | For amplification of acrRNA865 |
| | | without predicted native promtor for |
| | | insertion behind pBad |
| SEQ ID NO: 8 | DNA/RNA | For amplification of acrRNA1792 |
| | | without predicted native promtor for |
| | | insertion behind pBad |
| SEQ ID NO: 9 | DNA/RNA | For amplification of acrRNA1794 |
| | | without predicted native promtor for |
| | | insertion behind pBad |
| SEQ ID NO: 10 | DNA/RNA | Fragment comprising acrRNA773 |
| SEQ ID NO: 11 | DNA/RNA | Fragment comprising acrRNA865 |
| SEQ ID NO: 12 | DNA/RNA | Fragment comprising acrRNA1792 |
| SEQ ID NO: 13 | DNA/RNA | Fragment comprising acrRNA1794 |
| SEQ ID NO: 14 | DNA/RNA | Ors15 full repeat I-F repeat |
| SEQ ID NO: 15 | DNA/RNA | Ors16 full repeat I-F repeat |
| SEQ ID NO: 16 | DNA/RNA | Ors23 Synthetic acrRNA865 |
| | | (palindromic repeat ony) |
| SEQ ID NO: 17 | DNA/RNA | Ors24 Synthetic acrRNA865 |
| | | (palindromic repeat ony) |
| SEQ ID NO: 18 | DNA/RNA | Ors29 V-A repeat-repeat |
| SEQ ID NO: 19 | DNA/RNA | Ors30 V-A repeat-repeat |
| SEQ ID NO: 20 | DNA/RNA | I-A repeat |
| SEQ ID NO: 21 | DNA/RNA | I-A repeat |
| SEQ ID NO: 22 | DNA/RNA | I-A repeat |
| SEQ ID NO: 23 | DNA/RNA | I-A repeat |
| SEQ ID NO: 24 | DNA/RNA | I-A repeat |
| SEQ ID NO: 25 | DNA/RNA | I-A repeat |
| SEQ ID NO: 26 | DNA/RNA | I-A repeat |
| SEQ ID NO: 27 | DNA/RNA | I-A repeat |
| SEQ ID NO: 28 | DNA/RNA | I-A repeat |
| SEQ ID NO: 29 | DNA/RNA | I-A repeat |
| SEQ ID NO: 30 | DNA/RNA | I-A repeat |
| SEQ ID NO: 31 | DNA/RNA | I-A repeat |
| SEQ ID NO: 32 | DNA/RNA | I-A repeat |
| SEQ ID NO: 33 | DNA/RNA | I-A repeat |
| SEQ ID NO: 34 | DNA/RNA | I-A repeat |
| SEQ ID NO: 35 | DNA/RNA | I-A repeat |
| SEQ ID NO: 36 | DNA/RNA | I-A repeat |
| SEQ ID NO: 37 | DNA/RNA | I-A repeat |
| SEQ ID NO: 38 | DNA/RNA | I-A repeat |
| SEQ ID NO: 39 | DNA/RNA | I-A repeat |
| SEQ ID NO: 40 | DNA/RNA | I-A repeat |
| SEQ ID NO: 41 | DNA/RNA | I-B repeat |
| SEQ ID NO: 42 | DNA/RNA | I-B repeat |
| SEQ ID NO: 43 | DNA/RNA | I-B repeat |
| SEQ ID NO: 44 | DNA/RNA | I-B repeat |
| SEQ ID NO: 45 | DNA/RNA | I-B repeat |
| SEQ ID NO: 46 | DNA/RNA | I-B repeat |
| SEQ ID NO: 47 | DNA/RNA | I-B repeat |
| SEQ ID NO: 48 | DNA/RNA | I-B repeat |
| SEQ ID NO: 49 | DNA/RNA | I-B repeat |
| SEQ ID NO: 50 | DNA/RNA | I-B repeat |
| SEQ ID NO: 51 | DNA/RNA | I-B repeat |
| SEQ ID NO: 52 | DNA/RNA | I-B repeat |
| SEQ ID NO: 53 | DNA/RNA | I-B repeat |
| SEQ ID NO: 54 | DNA/RNA | I-B repeat |
| SEQ ID NO: 55 | DNA/RNA | I-B repeat |
| SEQ ID NO: 56 | DNA/RNA | I-B repeat |
| SEQ ID NO: 57 | DNA/RNA | I-B repeat |
| SEQ ID NO: 58 | DNA/RNA | I-B repeat |
| SEQ ID NO: 59 | DNA/RNA | I-B repeat |
| SEQ ID NO: 60 | DNA/RNA | I-B repeat |
| SEQ ID NO: 61 | DNA/RNA | I-B repeat |
| SEQ ID NO: 62 | DNA/RNA | I-B repeat |
| SEQ ID NO: 63 | DNA/RNA | I-B repeat |
| SEQ ID NO: 64 | DNA/RNA | I-B repeat |
| SEQ ID NO: 65 | DNA/RNA | I-B repeat |
| SEQ ID NO: 66 | DNA/RNA | I-B repeat |
| SEQ ID NO: 67 | DNA/RNA | I-B repeat |
| SEQ ID NO: 68 | DNA/RNA | I-B repeat |
| SEQ ID NO: 69 | DNA/RNA | I-B repeat |
| SEQ ID NO: 70 | DNA/RNA | I-B repeat |
| SEQ ID NO: 71 | DNA/RNA | I-B repeat |
| SEQ ID NO: 72 | DNA/RNA | I-B repeat |
| SEQ ID NO: 73 | DNA/RNA | I-B repeat |
| SEQ ID NO: 74 | DNA/RNA | I-B repeat |
| SEQ ID NO: 75 | DNA/RNA | I-B repeat |
| SEQ ID NO: 76 | DNA/RNA | I-B repeat |
| SEQ ID NO: 77 | DNA/RNA | I-B repeat |
| SEQ ID NO: 78 | DNA/RNA | I-B repeat |
| SEQ ID NO: 79 | DNA/RNA | I-B repeat |
| SEQ ID NO: 80 | DNA/RNA | I-B repeat |
| SEQ ID NO: 81 | DNA/RNA | I-B repeat |
| SEQ ID NO: 82 | DNA/RNA | I-B repeat |
| SEQ ID NO: 83 | DNA/RNA | I-B repeat |
| SEQ ID NO: 84 | DNA/RNA | I-B repeat |
| SEQ ID NO: 85 | DNA/RNA | I-B repeat |
| SEQ ID NO: 86 | DNA/RNA | I-B repeat |
| SEQ ID NO: 87 | DNA/RNA | I-B repeat |
| SEQ ID NO: 88 | DNA/RNA | I-B repeat |
| SEQ ID NO: 89 | DNA/RNA | I-B repeat |
| SEQ ID NO: 90 | DNA/RNA | I-B repeat |
| SEQ ID NO: 91 | DNA/RNA | I-B repeat |
| SEQ ID NO: 92 | DNA/RNA | I-B repeat |
| SEQ ID NO: 93 | DNA/RNA | I-B repeat |
| SEQ ID NO: 94 | DNA/RNA | I-B repeat |
| SEQ ID NO: 95 | DNA/RNA | I-B repeat |
| SEQ ID NO: 96 | DNA/RNA | I-B repeat |
| SEQ ID NO: 97 | DNA/RNA | I-B repeat |
| SEQ ID NO: 98 | DNA/RNA | I-B repeat |
| SEQ ID NO: 99 | DNA/RNA | I-B repeat |
| SEQ ID NO: 100 | DNA/RNA | I-B repeat |
| SEQ ID NO: 101 | DNA/RNA | I-B repeat |
| SEQ ID NO: 102 | DNA/RNA | I-B repeat |
| SEQ ID NO: 103 | DNA/RNA | I-B repeat |
| SEQ ID NO: 104 | DNA/RNA | I-B repeat |
| SEQ ID NO: 105 | DNA/RNA | I-B repeat |
| SEQ ID NO: 106 | DNA/RNA | I-B repeat |
| SEQ ID NO: 107 | DNA/RNA | I-B repeat |
| SEQ ID NO: 108 | DNA/RNA | I-B repeat |
| SEQ ID NO: 109 | DNA/RNA | I-B repeat |
| SEQ ID NO: 110 | DNA/RNA | I-B repeat |
| SEQ ID NO: 111 | DNA/RNA | I-B repeat |
| SEQ ID NO: 112 | DNA/RNA | I-B repeat |
| SEQ ID NO: 113 | DNA/RNA | I-B repeat |
| SEQ ID NO: 114 | DNA/RNA | I-B repeat |
| SEQ ID NO: 115 | DNA/RNA | I-B repeat |
| SEQ ID NO: 116 | DNA/RNA | I-B repeat |
| SEQ ID NO: 117 | DNA/RNA | I-B repeat |
| SEQ ID NO: 118 | DNA/RNA | I-B repeat |
| SEQ ID NO: 119 | DNA/RNA | I-B repeat |
| SEQ ID NO: 120 | DNA/RNA | I-B repeat |
| SEQ ID NO: 121 | DNA/RNA | I-B repeat |
| SEQ ID NO: 122 | DNA/RNA | I-B repeat |
| SEQ ID NO: 123 | DNA/RNA | I-B repeat |
| SEQ ID NO: 124 | DNA/RNA | I-B repeat |
| SEQ ID NO: 125 | DNA/RNA | I-B repeat |
| SEQ ID NO: 126 | DNA/RNA | I-B repeat |
| SEQ ID NO: 127 | DNA/RNA | I-B repeat |
| SEQ ID NO: 128 | DNA/RNA | I-B repeat |
| SEQ ID NO: 129 | DNA/RNA | I-B repeat |
| SEQ ID NO: 130 | DNA/RNA | I-B repeat |
| SEQ ID NO: 131 | DNA/RNA | I-B repeat |
| SEQ ID NO: 132 | DNA/RNA | I-B repeat |
| SEQ ID NO: 133 | DNA/RNA | I-B repeat |
| SEQ ID NO: 134 | DNA/RNA | I-B repeat |
| SEQ ID NO: 135 | DNA/RNA | I-B repeat |
| SEQ ID NO: 136 | DNA/RNA | I-B repeat |
| SEQ ID NO: 137 | DNA/RNA | I-B repeat |
| SEQ ID NO: 138 | DNA/RNA | I-B repeat |
| SEQ ID NO: 139 | DNA/RNA | I-B repeat |
| SEQ ID NO: 140 | DNA/RNA | I-B repeat |
| SEQ ID NO: 141 | DNA/RNA | I-B repeat |
| SEQ ID NO: 142 | DNA/RNA | I-B repeat |
| SEQ ID NO: 143 | DNA/RNA | I-B repeat |
| SEQ ID NO: 144 | DNA/RNA | I-B repeat |
| SEQ ID NO: 145 | DNA/RNA | I-B repeat |
| SEQ ID NO: 146 | DNA/RNA | I-B repeat |
| SEQ ID NO: 147 | DNA/RNA | I-B repeat |
| SEQ ID NO: 148 | DNA/RNA | I-B repeat |
| SEQ ID NO: 149 | DNA/RNA | I-B repeat |
| SEQ ID NO: 150 | DNA/RNA | I-B repeat |
| SEQ ID NO: 151 | DNA/RNA | I-C repeat |
| SEQ ID NO: 152 | DNA/RNA | I-C repeat |
| SEQ ID NO: 153 | DNA/RNA | I-C repeat |
| SEQ ID NO: 154 | DNA/RNA | I-C repeat |
| SEQ ID NO: 155 | DNA/RNA | I-C repeat |
| SEQ ID NO: 156 | DNA/RNA | I-C repeat |
| SEQ ID NO: 157 | DNA/RNA | I-C repeat |
| SEQ ID NO: 158 | DNA/RNA | I-C repeat |
| SEQ ID NO: 159 | DNA/RNA | I-C repeat |
| SEQ ID NO: 160 | DNA/RNA | I-C repeat |
| SEQ ID NO: 161 | DNA/RNA | I-C repeat |
| SEQ ID NO: 162 | DNA/RNA | I-C repeat |
| SEQ ID NO: 163 | DNA/RNA | I-C repeat |
| SEQ ID NO: 164 | DNA/RNA | I-C repeat |
| SEQ ID NO: 165 | DNA/RNA | I-C repeat |
| SEQ ID NO: 166 | DNA/RNA | I-C repeat |
| SEQ ID NO: 167 | DNA/RNA | I-C repeat |
| SEQ ID NO: 168 | DNA/RNA | I-C repeat |
| SEQ ID NO: 169 | DNA/RNA | I-C repeat |
| SEQ ID NO: 170 | DNA/RNA | I-C repeat |
| SEQ ID NO: 171 | DNA/RNA | I-C repeat |
| SEQ ID NO: 172 | DNA/RNA | I-C repeat |
| SEQ ID NO: 173 | DNA/RNA | I-C repeat |
| SEQ ID NO: 174 | DNA/RNA | I-C repeat |
| SEQ ID NO: 175 | DNA/RNA | I-C repeat |
| SEQ ID NO: 176 | DNA/RNA | I-C repeat |
| SEQ ID NO: 177 | DNA/RNA | I-C repeat |
| SEQ ID NO: 178 | DNA/RNA | I-C repeat |
| SEQ ID NO: 179 | DNA/RNA | I-C repeat |
| SEQ ID NO: 180 | DNA/RNA | I-C repeat |
| SEQ ID NO: 181 | DNA/RNA | I-C repeat |
| SEQ ID NO: 182 | DNA/RNA | I-C repeat |
| SEQ ID NO: 183 | DNA/RNA | I-C repeat |
| SEQ ID NO: 184 | DNA/RNA | I-C repeat |
| SEQ ID NO: 185 | DNA/RNA | I-C repeat |
| SEQ ID NO: 186 | DNA/RNA | I-C repeat |
| SEQ ID NO: 187 | DNA/RNA | I-C repeat |
| SEQ ID NO: 188 | DNA/RNA | I-C repeat |
| SEQ ID NO: 189 | DNA/RNA | I-C repeat |
| SEQ ID NO: 190 | DNA/RNA | I-C repeat |
| SEQ ID NO: 191 | DNA/RNA | I-C repeat |
| SEQ ID NO: 192 | DNA/RNA | I-C repeat |
| SEQ ID NO: 193 | DNA/RNA | I-C repeat |
| SEQ ID NO: 194 | DNA/RNA | I-C repeat |
| SEQ ID NO: 195 | DNA/RNA | I-C repeat |
| SEQ ID NO: 196 | DNA/RNA | I-C repeat |
| SEQ ID NO: 197 | DNA/RNA | I-C repeat |
| SEQ ID NO: 198 | DNA/RNA | I-C repeat |
| SEQ ID NO: 199 | DNA/RNA | I-C repeat |
| SEQ ID NO: 200 | DNA/RNA | I-C repeat |
| SEQ ID NO: 201 | DNA/RNA | I-C repeat |
| SEQ ID NO: 202 | DNA/RNA | I-C repeat |
| SEQ ID NO: 203 | DNA/RNA | I-C repeat |
| SEQ ID NO: 204 | DNA/RNA | I-C repeat |
| SEQ ID NO: 205 | DNA/RNA | I-C repeat |
| SEQ ID NO: 206 | DNA/RNA | I-C repeat |
| SEQ ID NO: 207 | DNA/RNA | I-C repeat |
| SEQ ID NO: 208 | DNA/RNA | I-C repeat |
| SEQ ID NO: 209 | DNA/RNA | I-C repeat |
| SEQ ID NO: 210 | DNA/RNA | I-C repeat |
| SEQ ID NO: 211 | DNA/RNA | I-C repeat |
| SEQ ID NO: 212 | DNA/RNA | I-C repeat |
| SEQ ID NO: 213 | DNA/RNA | I-C repeat |
| SEQ ID NO: 214 | DNA/RNA | I-C repeat |
| SEQ ID NO: 215 | DNA/RNA | I-C repeat |
| SEQ ID NO: 216 | DNA/RNA | I-C repeat |
| SEQ ID NO: 217 | DNA/RNA | I-C repeat |
| SEQ ID NO: 218 | DNA/RNA | I-C repeat |
| SEQ ID NO: 219 | DNA/RNA | I-C repeat |
| SEQ ID NO: 220 | DNA/RNA | I-C repeat |
| SEQ ID NO: 221 | DNA/RNA | I-C repeat |
| SEQ ID NO: 222 | DNA/RNA | I-C repeat |
| SEQ ID NO: 223 | DNA/RNA | I-C repeat |
| SEQ ID NO: 224 | DNA/RNA | I-C repeat |
| SEQ ID NO: 225 | DNA/RNA | I-C repeat |
| SEQ ID NO: 226 | DNA/RNA | I-C repeat |
| SEQ ID NO: 227 | DNA/RNA | I-C repeat |
| SEQ ID NO: 228 | DNA/RNA | I-C repeat |
| SEQ ID NO: 229 | DNA/RNA | I-C repeat |
| SEQ ID NO: 230 | DNA/RNA | I-C repeat |
| SEQ ID NO: 231 | DNA/RNA | I-C repeat |
| SEQ ID NO: 232 | DNA/RNA | I-C repeat |
| SEQ ID NO: 233 | DNA/RNA | I-C repeat |
| SEQ ID NO: 234 | DNA/RNA | I-C repeat |
| SEQ ID NO: 235 | DNA/RNA | I-C repeat |
| SEQ ID NO: 236 | DNA/RNA | I-C repeat |
| SEQ ID NO: 237 | DNA/RNA | I-C repeat |
| SEQ ID NO: 238 | DNA/RNA | I-C repeat |
| SEQ ID NO: 239 | DNA/RNA | I-C repeat |
| SEQ ID NO: 240 | DNA/RNA | I-C repeat |
| SEQ ID NO: 241 | DNA/RNA | I-C repeat |
| SEQ ID NO: 242 | DNA/RNA | I-C repeat |
| SEQ ID NO: 243 | DNA/RNA | I-C repeat |
| SEQ ID NO: 244 | DNA/RNA | I-C repeat |
| SEQ ID NO: 245 | DNA/RNA | I-D repeat |
| SEQ ID NO: 246 | DNA/RNA | I-D repeat |
| SEQ ID NO: 247 | DNA/RNA | I-D repeat |
| SEQ ID NO: 248 | DNA/RNA | I-D repeat |
| SEQ ID NO: 249 | DNA/RNA | I-D repeat |
| SEQ ID NO: 250 | DNA/RNA | I-D repeat |
| SEQ ID NO: 251 | DNA/RNA | I-D repeat |
| SEQ ID NO: 252 | DNA/RNA | I-D repeat |
| SEQ ID NO: 253 | DNA/RNA | I-D repeat |
| SEQ ID NO: 254 | DNA/RNA | I-D repeat |
| SEQ ID NO: 255 | DNA/RNA | I-D repeat |
| SEQ ID NO: 256 | DNA/RNA | I-D repeat |
| SEQ ID NO: 257 | DNA/RNA | I-D repeat |
| SEQ ID NO: 258 | DNA/RNA | I-D repeat |
| SEQ ID NO: 259 | DNA/RNA | I-D repeat |
| SEQ ID NO: 260 | DNA/RNA | I-D repeat |
| SEQ ID NO: 261 | DNA/RNA | I-D repeat |
| SEQ ID NO: 262 | DNA/RNA | I-D repeat |
| SEQ ID NO: 263 | DNA/RNA | I-D repeat |
| SEQ ID NO: 264 | DNA/RNA | I-D repeat |
| SEQ ID NO: 265 | DNA/RNA | I-D repeat |
| SEQ ID NO: 266 | DNA/RNA | I-D repeat |
| SEQ ID NO: 267 | DNA/RNA | I-D repeat |
| SEQ ID NO: 268 | DNA/RNA | I-D repeat |
| SEQ ID NO: 269 | DNA/RNA | I-D repeat |
| SEQ ID NO: 270 | DNA/RNA | I-D repeat |
| SEQ ID NO: 271 | DNA/RNA | I-D repeat |
| SEQ ID NO: 272 | DNA/RNA | I-D repeat |
| SEQ ID NO: 273 | DNA/RNA | I-D repeat |
| SEQ ID NO: 274 | DNA/RNA | I-D repeat |
| SEQ ID NO: 275 | DNA/RNA | I-D repeat |
| SEQ ID NO: 276 | DNA/RNA | I-D repeat |
| SEQ ID NO: 277 | DNA/RNA | I-D repeat |
| SEQ ID NO: 278 | DNA/RNA | I-D repeat |
| SEQ ID NO: 279 | DNA/RNA | I-D repeat |
| SEQ ID NO: 280 | DNA/RNA | I-E repeat |
| SEQ ID NO: 281 | DNA/RNA | I-E repeat |
| SEQ ID NO: 282 | DNA/RNA | I-E repeat |
| SEQ ID NO: 283 | DNA/RNA | I-E repeat |
| SEQ ID NO: 284 | DNA/RNA | I-E repeat |
| SEQ ID NO: 285 | DNA/RNA | I-E repeat |
| SEQ ID NO: 286 | DNA/RNA | I-E repeat |
| SEQ ID NO: 287 | DNA/RNA | I-E repeat |
| SEQ ID NO: 288 | DNA/RNA | I-E repeat |
| SEQ ID NO: 289 | DNA/RNA | I-E repeat |
| SEQ ID NO: 290 | DNA/RNA | I-E repeat |
| SEQ ID NO: 291 | DNA/RNA | I-E repeat |
| SEQ ID NO: 292 | DNA/RNA | I-E repeat |
| SEQ ID NO: 293 | DNA/RNA | I-E repeat |
| SEQ ID NO: 294 | DNA/RNA | I-E repeat |
| SEQ ID NO: 295 | DNA/RNA | I-E repeat |
| SEQ ID NO: 296 | DNA/RNA | I-E repeat |
| SEQ ID NO: 297 | DNA/RNA | I-E repeat |
| SEQ ID NO: 298 | DNA/RNA | I-E repeat |
| SEQ ID NO: 299 | DNA/RNA | I-E repeat |
| SEQ ID NO: 300 | DNA/RNA | I-E repeat |
| SEQ ID NO: 301 | DNA/RNA | I-E repeat |
| SEQ ID NO: 302 | DNA/RNA | I-E repeat |
| SEQ ID NO: 303 | DNA/RNA | I-E repeat |
| SEQ ID NO: 304 | DNA/RNA | I-E repeat |
| SEQ ID NO: 305 | DNA/RNA | I-E repeat |
| SEQ ID NO: 306 | DNA/RNA | I-E repeat |
| SEQ ID NO: 307 | DNA/RNA | I-E repeat |
| SEQ ID NO: 308 | DNA/RNA | I-E repeat |
| SEQ ID NO: 309 | DNA/RNA | I-E repeat |
| SEQ ID NO: 310 | DNA/RNA | I-E repeat |
| SEQ ID NO: 311 | DNA/RNA | I-E repeat |
| SEQ ID NO: 312 | DNA/RNA | I-E repeat |
| SEQ ID NO: 313 | DNA/RNA | I-E repeat |
| SEQ ID NO: 314 | DNA/RNA | I-F repeat |
| SEQ ID NO: 315 | DNA/RNA | I-F repeat |
| SEQ ID NO: 316 | DNA/RNA | I-F repeat |
| SEQ ID NO: 317 | DNA/RNA | I-F repeat |
| SEQ ID NO: 318 | DNA/RNA | I-F repeat |
| SEQ ID NO: 319 | DNA/RNA | I-F repeat |
| SEQ ID NO: 320 | DNA/RNA | I-F repeat |
| SEQ ID NO: 321 | DNA/RNA | I-F repeat |
| SEQ ID NO: 322 | DNA/RNA | I-F repeat |
| SEQ ID NO: 323 | DNA/RNA | I-F repeat |
| SEQ ID NO: 324 | DNA/RNA | I-F repeat |
| SEQ ID NO: 325 | DNA/RNA | I-F repeat |
| SEQ ID NO: 326 | DNA/RNA | I-F repeat |
| SEQ ID NO: 327 | DNA/RNA | I-F repeat |
| SEQ ID NO: 328 | DNA/RNA | I-F repeat |
| SEQ ID NO: 329 | DNA/RNA | I-F repeat |
| SEQ ID NO: 330 | DNA/RNA | I-F repeat |
| SEQ ID NO: 331 | DNA/RNA | I-F repeat |
| SEQ ID NO: 332 | DNA/RNA | I-F repeat |
| SEQ ID NO: 333 | DNA/RNA | I-F repeat |
| SEQ ID NO: 334 | DNA/RNA | I-F repeat |
| SEQ ID NO: 335 | DNA/RNA | I-F repeat |
| SEQ ID NO: 336 | DNA/RNA | I-G repeat |
| SEQ ID NO: 337 | DNA/RNA | I-G repeat |
| SEQ ID NO: 338 | DNA/RNA | I-G repeat |
| SEQ ID NO: 339 | DNA/RNA | I-G repeat |
| SEQ ID NO: 340 | DNA/RNA | I-G repeat |
| SEQ ID NO: 341 | DNA/RNA | I-G repeat |
| SEQ ID NO: 342 | DNA/RNA | I-G repeat |
| SEQ ID NO: 343 | DNA/RNA | I-G repeat |
| SEQ ID NO: 344 | DNA/RNA | I-G repeat |
| SEQ ID NO: 345 | DNA/RNA | I-G repeat |
| SEQ ID NO: 346 | DNA/RNA | I-G repeat |
| SEQ ID NO: 347 | DNA/RNA | I-G repeat |
| SEQ ID NO: 348 | DNA/RNA | I-G repeat |
| SEQ ID NO: 349 | DNA/RNA | I-G repeat |
| SEQ ID NO: 350 | DNA/RNA | I-G repeat |
| SEQ ID NO: 351 | DNA/RNA | I-G repeat |
| SEQ ID NO: 352 | DNA/RNA | I-G repeat |
| SEQ ID NO: 353 | DNA/RNA | I-G repeat |
| SEQ ID NO: 354 | DNA/RNA | I-G repeat |
| SEQ ID NO: 355 | DNA/RNA | I-G repeat |
| SEQ ID NO: 356 | DNA/RNA | I-G repeat |
| SEQ ID NO: 357 | DNA/RNA | I-G repeat |
| SEQ ID NO: 358 | DNA/RNA | I-G repeat |
| SEQ ID NO: 359 | DNA/RNA | I-G repeat |
| SEQ ID NO: 360 | DNA/RNA | I-G repeat |
| SEQ ID NO: 361 | DNA/RNA | I-G repeat |
| SEQ ID NO: 362 | DNA/RNA | I-G repeat |
| SEQ ID NO: 363 | DNA/RNA | I-G repeat |
| SEQ ID NO: 364 | DNA/RNA | I-G repeat |
| SEQ ID NO: 365 | DNA/RNA | I-G repeat |
| SEQ ID NO: 366 | DNA/RNA | I-G repeat |
| SEQ ID NO: 367 | DNA/RNA | I-G repeat |
| SEQ ID NO: 368 | DNA/RNA | I-G repeat |
| SEQ ID NO: 369 | DNA/RNA | I-G repeat |
| SEQ ID NO: 370 | DNA/RNA | I-G repeat |
| SEQ ID NO: 371 | DNA/RNA | I-G repeat |
| SEQ ID NO: 372 | DNA/RNA | I-G repeat |
| SEQ ID NO: 373 | DNA/RNA | I-G repeat |
| SEQ ID NO: 374 | DNA/RNA | I-G repeat |
| SEQ ID NO: 375 | DNA/RNA | I-G repeat |
| SEQ ID NO: 376 | DNA/RNA | I-G repeat |
| SEQ ID NO: 377 | DNA/RNA | I-G repeat |
| SEQ ID NO: 378 | DNA/RNA | I-G repeat |
| SEQ ID NO: 379 | DNA/RNA | I-G repeat |
| SEQ ID NO: 380 | DNA/RNA | I-G repeat |
| SEQ ID NO: 381 | DNA/RNA | I-G repeat |
| SEQ ID NO: 382 | DNA/RNA | I-G repeat |
| SEQ ID NO: 383 | DNA/RNA | I-G repeat |
| SEQ ID NO: 384 | DNA/RNA | I-G repeat |
| SEQ ID NO: 385 | DNA/RNA | I-G repeat |
| SEQ ID NO: 386 | DNA/RNA | I-G repeat |
| SEQ ID NO: 387 | DNA/RNA | I-G repeat |
| SEQ ID NO: 388 | DNA/RNA | I-G repeat |
| SEQ ID NO: 389 | DNA/RNA | I-G repeat |
| SEQ ID NO: 390 | DNA/RNA | I-G repeat |
| SEQ ID NO: 391 | DNA/RNA | II-A repeat |
| SEQ ID NO: 392 | DNA/RNA | II-A repeat |
| SEQ ID NO: 393 | DNA/RNA | II-A repeat |
| SEQ ID NO: 394 | DNA/RNA | II-A repeat |
| SEQ ID NO: 395 | DNA/RNA | II-A repeat |
| SEQ ID NO: 396 | DNA/RNA | II-A repeat |
| SEQ ID NO: 397 | DNA/RNA | II-A repeat |
| SEQ ID NO: 398 | DNA/RNA | II-A repeat |
| SEQ ID NO: 399 | DNA/RNA | II-A repeat |
| SEQ ID NO: 400 | DNA/RNA | II-A repeat |
| SEQ ID NO: 401 | DNA/RNA | II-A repeat |
| SEQ ID NO: 402 | DNA/RNA | II-A repeat |
| SEQ ID NO: 403 | DNA/RNA | II-A repeat |
| SEQ ID NO: 404 | DNA/RNA | II-A repeat |
| SEQ ID NO: 405 | DNA/RNA | II-A repeat |
| SEQ ID NO: 406 | DNA/RNA | II-A repeat |
| SEQ ID NO: 407 | DNA/RNA | II-A repeat |
| SEQ ID NO: 408 | DNA/RNA | II-A repeat |
| SEQ ID NO: 409 | DNA/RNA | II-A repeat |
| SEQ ID NO: 410 | DNA/RNA | II-A repeat |
| SEQ ID NO: 411 | DNA/RNA | II-A repeat |
| SEQ ID NO: 412 | DNA/RNA | II-A repeat |
| SEQ ID NO: 413 | DNA/RNA | II-A repeat |
| SEQ ID NO: 414 | DNA/RNA | II-A repeat |
| SEQ ID NO: 415 | DNA/RNA | II-A repeat |
| SEQ ID NO: 416 | DNA/RNA | II-A repeat |
| SEQ ID NO: 417 | DNA/RNA | II-A repeat |
| SEQ ID NO: 418 | DNA/RNA | II-A repeat |
| SEQ ID NO: 419 | DNA/RNA | II-A repeat |
| SEQ ID NO: 420 | DNA/RNA | II-A repeat |
| SEQ ID NO: 421 | DNA/RNA | II-A repeat |
| SEQ ID NO: 422 | DNA/RNA | II-A repeat |
| SEQ ID NO: 423 | DNA/RNA | II-A repeat |
| SEQ ID NO: 424 | DNA/RNA | II-A repeat |
| SEQ ID NO: 425 | DNA/RNA | II-A repeat |
| SEQ ID NO: 426 | DNA/RNA | II-A repeat |
| SEQ ID NO: 427 | DNA/RNA | II-A repeat |
| SEQ ID NO: 428 | DNA/RNA | II-A repeat |
| SEQ ID NO: 429 | DNA/RNA | II-A repeat |
| SEQ ID NO: 430 | DNA/RNA | II-A repeat |
| SEQ ID NO: 431 | DNA/RNA | II-A repeat |
| SEQ ID NO: 432 | DNA/RNA | II-A repeat |
| SEQ ID NO: 433 | DNA/RNA | II-A repeat |
| SEQ ID NO: 434 | DNA/RNA | II-A repeat |
| SEQ ID NO: 435 | DNA/RNA | II-A repeat |
| SEQ ID NO: 436 | DNA/RNA | II-A repeat |
| SEQ ID NO: 437 | DNA/RNA | II-A repeat |
| SEQ ID NO: 438 | DNA/RNA | II-A repeat |
| SEQ ID NO: 439 | DNA/RNA | II-A repeat |
| SEQ ID NO: 440 | DNA/RNA | II-A repeat |
| SEQ ID NO: 441 | DNA/RNA | II-A repeat |
| SEQ ID NO: 442 | DNA/RNA | II-A repeat |
| SEQ ID NO: 443 | DNA/RNA | II-A repeat |
| SEQ ID NO: 444 | DNA/RNA | II-A repeat |
| SEQ ID NO: 445 | DNA/RNA | II-A repeat |
| SEQ ID NO: 446 | DNA/RNA | II-A repeat |
| SEQ ID NO: 447 | DNA/RNA | II-A repeat |
| SEQ ID NO: 448 | DNA/RNA | II-A repeat |
| SEQ ID NO: 449 | DNA/RNA | II-A repeat |
| SEQ ID NO: 450 | DNA/RNA | II-A repeat |
| SEQ ID NO: 451 | DNA/RNA | II-A repeat |
| SEQ ID NO: 452 | DNA/RNA | II-A repeat |
| SEQ ID NO: 453 | DNA/RNA | II-A repeat |
| SEQ ID NO: 454 | DNA/RNA | II-A repeat |
| SEQ ID NO: 455 | DNA/RNA | II-A repeat |
| SEQ ID NO: 456 | DNA/RNA | II-A repeat |
| SEQ ID NO: 457 | DNA/RNA | II-A repeat |
| SEQ ID NO: 458 | DNA/RNA | II-A repeat |
| SEQ ID NO: 459 | DNA/RNA | II-A repeat |
| SEQ ID NO: 460 | DNA/RNA | II-A repeat |
| SEQ ID NO: 461 | DNA/RNA | II-A repeat |
| SEQ ID NO: 462 | DNA/RNA | II-A repeat |
| SEQ ID NO: 463 | DNA/RNA | II-A repeat |
| SEQ ID NO: 464 | DNA/RNA | II-A repeat |
| SEQ ID NO: 465 | DNA/RNA | II-A repeat |
| SEQ ID NO: 466 | DNA/RNA | II-A repeat |
| SEQ ID NO: 467 | DNA/RNA | II-A repeat |
| SEQ ID NO: 468 | DNA/RNA | II-A repeat |
| SEQ ID NO: 469 | DNA/RNA | II-A repeat |
| SEQ ID NO: 470 | DNA/RNA | II-A repeat |
| SEQ ID NO: 471 | DNA/RNA | II-A repeat |
| SEQ ID NO: 472 | DNA/RNA | II-A repeat |
| SEQ ID NO: 473 | DNA/RNA | II-A repeat |
| SEQ ID NO: 474 | DNA/RNA | II-A repeat |
| SEQ ID NO: 475 | DNA/RNA | II-A repeat |
| SEQ ID NO: 476 | DNA/RNA | II-A repeat |
| SEQ ID NO: 477 | DNA/RNA | II-B repeat |
| SEQ ID NO: 478 | DNA/RNA | II-B repeat |
| SEQ ID NO: 479 | DNA/RNA | II-B repeat |
| SEQ ID NO: 480 | DNA/RNA | II-B repeat |
| SEQ ID NO: 481 | DNA/RNA | II-B repeat |
| SEQ ID NO: 482 | DNA/RNA | II-B repeat |
| SEQ ID NO: 483 | DNA/RNA | II-B repeat |
| SEQ ID NO: 484 | DNA/RNA | II-B repeat |
| SEQ ID NO: 485 | DNA/RNA | II-B repeat |
| SEQ ID NO: 486 | DNA/RNA | II-B repeat |
| SEQ ID NO: 487 | DNA/RNA | II-B repeat |
| SEQ ID NO: 488 | DNA/RNA | II-B repeat |
| SEQ ID NO: 489 | DNA/RNA | II-C repeat |
| SEQ ID NO: 490 | DNA/RNA | II-C repeat |
| SEQ ID NO: 491 | DNA/RNA | II-C repeat |
| SEQ ID NO: 492 | DNA/RNA | II-C repeat |
| SEQ ID NO: 493 | DNA/RNA | II-C repeat |
| SEQ ID NO: 494 | DNA/RNA | II-C repeat |
| SEQ ID NO: 495 | DNA/RNA | II-C repeat |
| SEQ ID NO: 496 | DNA/RNA | II-C repeat |
| SEQ ID NO: 497 | DNA/RNA | II-C repeat |
| SEQ ID NO: 498 | DNA/RNA | II-C repeat |
| SEQ ID NO: 499 | DNA/RNA | II-C repeat |
| SEQ ID NO: 500 | DNA/RNA | II-C repeat |
| SEQ ID NO: 501 | DNA/RNA | II-C repeat |
| SEQ ID NO: 502 | DNA/RNA | II-C repeat |
| SEQ ID NO: 503 | DNA/RNA | II-C repeat |
| SEQ ID NO: 504 | DNA/RNA | II-C repeat |
| SEQ ID NO: 505 | DNA/RNA | II-C repeat |
| SEQ ID NO: 506 | DNA/RNA | II-C repeat |
| SEQ ID NO: 507 | DNA/RNA | II-C repeat |
| SEQ ID NO: 508 | DNA/RNA | II-C repeat |
| SEQ ID NO: 509 | DNA/RNA | II-C repeat |
| SEQ ID NO: 510 | DNA/RNA | II-C repeat |
| SEQ ID NO: 511 | DNA/RNA | II-C repeat |
| SEQ ID NO: 512 | DNA/RNA | II-C repeat |
| SEQ ID NO: 513 | DNA/RNA | II-C repeat |
| SEQ ID NO: 514 | DNA/RNA | II-C repeat |
| SEQ ID NO: 515 | DNA/RNA | II-C repeat |
| SEQ ID NO: 516 | DNA/RNA | II-C repeat |
| SEQ ID NO: 517 | DNA/RNA | II-C repeat |
| SEQ ID NO: 518 | DNA/RNA | II-C repeat |
| SEQ ID NO: 519 | DNA/RNA | II-C repeat |
| SEQ ID NO: 520 | DNA/RNA | II-C repeat |
| SEQ ID NO: 521 | DNA/RNA | II-C repeat |
| SEQ ID NO: 522 | DNA/RNA | II-C repeat |
| SEQ ID NO: 523 | DNA/RNA | II-C repeat |
| SEQ ID NO: 524 | DNA/RNA | II-C repeat |
| SEQ ID NO: 525 | DNA/RNA | II-C repeat |
| SEQ ID NO: 526 | DNA/RNA | II-C repeat |
| SEQ ID NO: 527 | DNA/RNA | II-C repeat |
| SEQ ID NO: 528 | DNA/RNA | II-C repeat |
| SEQ ID NO: 529 | DNA/RNA | II-C repeat |
| SEQ ID NO: 530 | DNA/RNA | III-A repeat |
| SEQ ID NO: 531 | DNA/RNA | III-A repeat |
| SEQ ID NO: 532 | DNA/RNA | III-A repeat |
| SEQ ID NO: 533 | DNA/RNA | III-A repeat |
| SEQ ID NO: 534 | DNA/RNA | III-A repeat |
| SEQ ID NO: 535 | DNA/RNA | III-A repeat |
| SEQ ID NO: 536 | DNA/RNA | III-A repeat |
| SEQ ID NO: 537 | DNA/RNA | III-A repeat |
| SEQ ID NO: 538 | DNA/RNA | III-A repeat |
| SEQ ID NO: 539 | DNA/RNA | III-A repeat |
| SEQ ID NO: 540 | DNA/RNA | III-A repeat |
| SEQ ID NO: 541 | DNA/RNA | III-A repeat |
| SEQ ID NO: 542 | DNA/RNA | III-A repeat |
| SEQ ID NO: 543 | DNA/RNA | III-A repeat |
| SEQ ID NO: 544 | DNA/RNA | III-A repeat |
| SEQ ID NO: 545 | DNA/RNA | III-A repeat |
| SEQ ID NO: 546 | DNA/RNA | III-A repeat |
| SEQ ID NO: 547 | DNA/RNA | III-A repeat |
| SEQ ID NO: 548 | DNA/RNA | III-A repeat |
| SEQ ID NO: 549 | DNA/RNA | III-A repeat |
| SEQ ID NO: 550 | DNA/RNA | III-A repeat |
| SEQ ID NO: 551 | DNA/RNA | III-A repeat |
| SEQ ID NO: 552 | DNA/RNA | III-A repeat |
| SEQ ID NO: 553 | DNA/RNA | III-A repeat |
| SEQ ID NO: 554 | DNA/RNA | III-A repeat |
| SEQ ID NO: 555 | DNA/RNA | III-A repeat |
| SEQ ID NO: 556 | DNA/RNA | III-A repeat |
| SEQ ID NO: 557 | DNA/RNA | III-A repeat |
| SEQ ID NO: 558 | DNA/RNA | III-A repeat |
| SEQ ID NO: 559 | DNA/RNA | III-A repeat |
| SEQ ID NO: 560 | DNA/RNA | III-A repeat |
| SEQ ID NO: 561 | DNA/RNA | III-A repeat |
| SEQ ID NO: 562 | DNA/RNA | III-A repeat |
| SEQ ID NO: 563 | DNA/RNA | III-A repeat |
| SEQ ID NO: 564 | DNA/RNA | III-A repeat |
| SEQ ID NO: 565 | DNA/RNA | III-A repeat |
| SEQ ID NO: 566 | DNA/RNA | III-A repeat |
| SEQ ID NO: 567 | DNA/RNA | III-A repeat |
| SEQ ID NO: 568 | DNA/RNA | III-A repeat |
| SEQ ID NO: 569 | DNA/RNA | III-A repeat |
| SEQ ID NO: 570 | DNA/RNA | III-A repeat |
| SEQ ID NO: 571 | DNA/RNA | III-A repeat |
| SEQ ID NO: 572 | DNA/RNA | III-A repeat |
| SEQ ID NO: 573 | DNA/RNA | III-A repeat |
| SEQ ID NO: 574 | DNA/RNA | III-A repeat |
| SEQ ID NO: 575 | DNA/RNA | III-A repeat |
| SEQ ID NO: 576 | DNA/RNA | III-A repeat |
| SEQ ID NO: 577 | DNA/RNA | III-A repeat |
| SEQ ID NO: 578 | DNA/RNA | III-A repeat |
| SEQ ID NO: 579 | DNA/RNA | III-A repeat |
| SEQ ID NO: 580 | DNA/RNA | III-A repeat |
| SEQ ID NO: 581 | DNA/RNA | III-A repeat |
| SEQ ID NO: 582 | DNA/RNA | III-A repeat |
| SEQ ID NO: 583 | DNA/RNA | III-A repeat |
| SEQ ID NO: 584 | DNA/RNA | III-A repeat |
| SEQ ID NO: 585 | DNA/RNA | III-A repeat |
| SEQ ID NO: 586 | DNA/RNA | III-A repeat |
| SEQ ID NO: 587 | DNA/RNA | III-A repeat |
| SEQ ID NO: 588 | DNA/RNA | III-A repeat |
| SEQ ID NO: 589 | DNA/RNA | III-A repeat |
| SEQ ID NO: 590 | DNA/RNA | III-A repeat |
| SEQ ID NO: 591 | DNA/RNA | III-A repeat |
| SEQ ID NO: 592 | DNA/RNA | III-A repeat |
| SEQ ID NO: 593 | DNA/RNA | III-A repeat |
| SEQ ID NO: 594 | DNA/RNA | III-A repeat |
| SEQ ID NO: 595 | DNA/RNA | III-A repeat |
| SEQ ID NO: 596 | DNA/RNA | III-A repeat |
| SEQ ID NO: 597 | DNA/RNA | III-A repeat |
| SEQ ID NO: 598 | DNA/RNA | III-A repeat |
| SEQ ID NO: 599 | DNA/RNA | III-A repeat |
| SEQ ID NO: 600 | DNA/RNA | III-A repeat |
| SEQ ID NO: 601 | DNA/RNA | III-A repeat |
| SEQ ID NO: 602 | DNA/RNA | III-A repeat |
| SEQ ID NO: 603 | DNA/RNA | III-A repeat |
| SEQ ID NO: 604 | DNA/RNA | III-A repeat |
| SEQ ID NO: 605 | DNA/RNA | III-A repeat |
| SEQ ID NO: 606 | DNA/RNA | III-A repeat |
| SEQ ID NO: 607 | DNA/RNA | III-A repeat |
| SEQ ID NO: 608 | DNA/RNA | III-A repeat |
| SEQ ID NO: 609 | DNA/RNA | III-A repeat |
| SEQ ID NO: 610 | DNA/RNA | III-A repeat |
| SEQ ID NO: 611 | DNA/RNA | III-A repeat |
| SEQ ID NO: 612 | DNA/RNA | III-A repeat |
| SEQ ID NO: 613 | DNA/RNA | III-A repeat |
| SEQ ID NO: 614 | DNA/RNA | III-A repeat |
| SEQ ID NO: 615 | DNA/RNA | III-A repeat |
| SEQ ID NO: 616 | DNA/RNA | III-A repeat |
| SEQ ID NO: 617 | DNA/RNA | III-A repeat |
| SEQ ID NO: 618 | DNA/RNA | III-A repeat |
| SEQ ID NO: 619 | DNA/RNA | III-A repeat |
| SEQ ID NO: 620 | DNA/RNA | III-A repeat |
| SEQ ID NO: 621 | DNA/RNA | III-A repeat |
| SEQ ID NO: 622 | DNA/RNA | III-A repeat |
| SEQ ID NO: 623 | DNA/RNA | III-B repeat |
| SEQ ID NO: 624 | DNA/RNA | III-B repeat |
| SEQ ID NO: 625 | DNA/RNA | III-B repeat |
| SEQ ID NO: 626 | DNA/RNA | III-B repeat |
| SEQ ID NO: 627 | DNA/RNA | III-B repeat |
| SEQ ID NO: 628 | DNA/RNA | III-B repeat |
| SEQ ID NO: 629 | DNA/RNA | III-B repeat |
| SEQ ID NO: 630 | DNA/RNA | III-B repeat |
| SEQ ID NO: 631 | DNA/RNA | III-B repeat |
| SEQ ID NO: 632 | DNA/RNA | III-B repeat |
| SEQ ID NO: 633 | DNA/RNA | III-B repeat |
| SEQ ID NO: 634 | DNA/RNA | III-B repeat |
| SEQ ID NO: 635 | DNA/RNA | III-B repeat |
| SEQ ID NO: 636 | DNA/RNA | III-B repeat |
| SEQ ID NO: 637 | DNA/RNA | III-B repeat |
| SEQ ID NO: 638 | DNA/RNA | III-B repeat |
| SEQ ID NO: 639 | DNA/RNA | III-B repeat |
| SEQ ID NO: 640 | DNA/RNA | III-B repeat |
| SEQ ID NO: 641 | DNA/RNA | III-B repeat |
| SEQ ID NO: 642 | DNA/RNA | III-B repeat |
| SEQ ID NO: 643 | DNA/RNA | III-B repeat |
| SEQ ID NO: 644 | DNA/RNA | III-B repeat |
| SEQ ID NO: 645 | DNA/RNA | III-B repeat |
| SEQ ID NO: 646 | DNA/RNA | III-B repeat |
| SEQ ID NO: 647 | DNA/RNA | III-B repeat |
| SEQ ID NO: 648 | DNA/RNA | III-B repeat |
| SEQ ID NO: 649 | DNA/RNA | III-B repeat |
| SEQ ID NO: 650 | DNA/RNA | III-B repeat |
| SEQ ID NO: 651 | DNA/RNA | III-B repeat |
| SEQ ID NO: 652 | DNA/RNA | III-B repeat |
| SEQ ID NO: 653 | DNA/RNA | III-B repeat |
| SEQ ID NO: 654 | DNA/RNA | III-B repeat |
| SEQ ID NO: 655 | DNA/RNA | III-B repeat |
| SEQ ID NO: 656 | DNA/RNA | III-B repeat |
| SEQ ID NO: 657 | DNA/RNA | III-B repeat |
| SEQ ID NO: 658 | DNA/RNA | III-B repeat |
| SEQ ID NO: 659 | DNA/RNA | III-B repeat |
| SEQ ID NO: 660 | DNA/RNA | III-B repeat |
| SEQ ID NO: 661 | DNA/RNA | III-B repeat |
| SEQ ID NO: 662 | DNA/RNA | III-B repeat |
| SEQ ID NO: 663 | DNA/RNA | III-B repeat |
| SEQ ID NO: 664 | DNA/RNA | III-B repeat |
| SEQ ID NO: 665 | DNA/RNA | III-B repeat |
| SEQ ID NO: 666 | DNA/RNA | III-B repeat |
| SEQ ID NO: 667 | DNA/RNA | III-B repeat |
| SEQ ID NO: 668 | DNA/RNA | III-B repeat |
| SEQ ID NO: 669 | DNA/RNA | III-B repeat |
| SEQ ID NO: 670 | DNA/RNA | III-B repeat |
| SEQ ID NO: 671 | DNA/RNA | III-B repeat |
| SEQ ID NO: 672 | DNA/RNA | III-B repeat |
| SEQ ID NO: 673 | DNA/RNA | III-B repeat |
| SEQ ID NO: 674 | DNA/RNA | III-B repeat |
| SEQ ID NO: 675 | DNA/RNA | III-B repeat |
| SEQ ID NO: 676 | DNA/RNA | III-B repeat |
| SEQ ID NO: 677 | DNA/RNA | III-C repeat |
| SEQ ID NO: 678 | DNA/RNA | III-C repeat |
| SEQ ID NO: 679 | DNA/RNA | III-C repeat |
| SEQ ID NO: 680 | DNA/RNA | III-D repeat |
| SEQ ID NO: 681 | DNA/RNA | III-D repeat |
| SEQ ID NO: 682 | DNA/RNA | III-D repeat |
| SEQ ID NO: 683 | DNA/RNA | III-D repeat |
| SEQ ID NO: 684 | DNA/RNA | III-D repeat |
| SEQ ID NO: 685 | DNA/RNA | III-D repeat |
| SEQ ID NO: 686 | DNA/RNA | III-D repeat |
| SEQ ID NO: 687 | DNA/RNA | III-D repeat |
| SEQ ID NO: 688 | DNA/RNA | III-D repeat |
| SEQ ID NO: 689 | DNA/RNA | III-D repeat |
| SEQ ID NO: 690 | DNA/RNA | III-D repeat |
| SEQ ID NO: 691 | DNA/RNA | III-D repeat |
| SEQ ID NO: 692 | DNA/RNA | III-D repeat |
| SEQ ID NO: 693 | DNA/RNA | III-D repeat |
| SEQ ID NO: 694 | DNA/RNA | III-D repeat |
| SEQ ID NO: 695 | DNA/RNA | III-D repeat |
| SEQ ID NO: 696 | DNA/RNA | III-D repeat |
| SEQ ID NO: 697 | DNA/RNA | III-D repeat |
| SEQ ID NO: 698 | DNA/RNA | III-D repeat |
| SEQ ID NO: 699 | DNA/RNA | III-D repeat |
| SEQ ID NO: 700 | DNA/RNA | III-D repeat |
| SEQ ID NO: 701 | DNA/RNA | III-D repeat |
| SEQ ID NO: 702 | DNA/RNA | III-D repeat |
| SEQ ID NO: 703 | DNA/RNA | III-D repeat |
| SEQ ID NO: 704 | DNA/RNA | III-D repeat |
| SEQ ID NO: 705 | DNA/RNA | III-D repeat |
| SEQ ID NO: 706 | DNA/RNA | III-D repeat |
| SEQ ID NO: 707 | DNA/RNA | III-D repeat |
| SEQ ID NO: 708 | DNA/RNA | III-D repeat |
| SEQ ID NO: 709 | DNA/RNA | III-D repeat |
| SEQ ID NO: 710 | DNA/RNA | III-D repeat |
| SEQ ID NO: 711 | DNA/RNA | III-D repeat |
| SEQ ID NO: 712 | DNA/RNA | III-D repeat |
| SEQ ID NO: 713 | DNA/RNA | III-D repeat |
| SEQ ID NO: 714 | DNA/RNA | III-D repeat |
| SEQ ID NO: 715 | DNA/RNA | III-D repeat |
| SEQ ID NO: 716 | DNA/RNA | III-D repeat |
| SEQ ID NO: 717 | DNA/RNA | III-D repeat |
| SEQ ID NO: 718 | DNA/RNA | III-D repeat |
| SEQ ID NO: 719 | DNA/RNA | III-D repeat |
| SEQ ID NO: 720 | DNA/RNA | III-D repeat |
| SEQ ID NO: 721 | DNA/RNA | III-D repeat |
| SEQ ID NO: 722 | DNA/RNA | III-D repeat |
| SEQ ID NO: 723 | DNA/RNA | III-E repeat |
| SEQ ID NO: 724 | DNA/RNA | III-E repeat |
| SEQ ID NO: 725 | DNA/RNA | III-E repeat |
| SEQ ID NO: 726 | DNA/RNA | III-E repeat |
| SEQ ID NO: 727 | DNA/RNA | III-E repeat |
| SEQ ID NO: 728 | DNA/RNA | III-E repeat |
| SEQ ID NO: 729 | DNA/RNA | III-E repeat |
| SEQ ID NO: 730 | DNA/RNA | III-E repeat |
| SEQ ID NO: 731 | DNA/RNA | III-F repeat |
| SEQ ID NO: 732 | DNA/RNA | III-F repeat |
| SEQ ID NO: 733 | DNA/RNA | III-F repeat |
| SEQ ID NO: 734 | DNA/RNA | III-F repeat |
| SEQ ID NO: 735 | DNA/RNA | III-F repeat |
| SEQ ID NO: 736 | DNA/RNA | III-F repeat |
| SEQ ID NO: 737 | DNA/RNA | III-F repeat |
| SEQ ID NO: 738 | DNA/RNA | IV-A1 repeat |
| SEQ ID NO: 739 | DNA/RNA | IV-A1 repeat |
| SEQ ID NO: 740 | DNA/RNA | IV-A1 repeat |
| SEQ ID NO: 741 | DNA/RNA | IV-A1 repeat |
| SEQ ID NO: 742 | DNA/RNA | IV-A1 repeat |
| SEQ ID NO: 743 | DNA/RNA | IV-A1 repeat |
| SEQ ID NO: 744 | DNA/RNA | IV-A1 repeat |
| SEQ ID NO: 745 | DNA/RNA | IV-A1 repeat |
| SEQ ID NO: 746 | DNA/RNA | IV-A1 repeat |
| SEQ ID NO: 747 | DNA/RNA | IV-A1 repeat |
| SEQ ID NO: 748 | DNA/RNA | IV-A1 repeat |
| SEQ ID NO: 749 | DNA/RNA | IV-A1 repeat |
| SEQ ID NO: 750 | DNA/RNA | IV-A1 repeat |
| SEQ ID NO: 751 | DNA/RNA | IV-A1 repeat |
| SEQ ID NO: 752 | DNA/RNA | IV-A1 repeat |
| SEQ ID NO: 753 | DNA/RNA | IV-A1 repeat |
| SEQ ID NO: 754 | DNA/RNA | IV-A1 repeat |
| SEQ ID NO: 755 | DNA/RNA | IV-A1 repeat |
| SEQ ID NO: 756 | DNA/RNA | IV-A1 repeat |
| SEQ ID NO: 757 | DNA/RNA | IV-A1 repeat |
| SEQ ID NO: 758 | DNA/RNA | IV-A1 repeat |
| SEQ ID NO: 759 | DNA/RNA | IV-A1 repeat |
| SEQ ID NO: 760 | DNA/RNA | IV-A2 repeat |
| SEQ ID NO: 761 | DNA/RNA | IV-A2 repeat |
| SEQ ID NO: 762 | DNA/RNA | IV-A2 repeat |
| SEQ ID NO: 763 | DNA/RNA | IV-A2 repeat |
| SEQ ID NO: 764 | DNA/RNA | IV-A2 repeat |
| SEQ ID NO: 765 | DNA/RNA | IV-A2 repeat |
| SEQ ID NO: 766 | DNA/RNA | IV-A2 repeat |
| SEQ ID NO: 767 | DNA/RNA | IV-A3 repeat |
| SEQ ID NO: 768 | DNA/RNA | IV-A3 repeat |
| SEQ ID NO: 769 | DNA/RNA | IV-A3 repeat |
| SEQ ID NO: 770 | DNA/RNA | IV-A3 repeat |
| SEQ ID NO: 771 | DNA/RNA | IV-A3 repeat |
| SEQ ID NO: 772 | DNA/RNA | IV-A3 repeat |
| SEQ ID NO: 773 | DNA/RNA | IV-A3 repeat |
| SEQ ID NO: 774 | DNA/RNA | IV-A3 repeat |
| SEQ ID NO: 775 | DNA/RNA | IV-B repeat |
| SEQ ID NO: 776 | DNA/RNA | IV-B repeat |
| SEQ ID NO: 777 | DNA/RNA | IV-B repeat |
| SEQ ID NO: 778 | DNA/RNA | IV-B repeat |
| SEQ ID NO: 779 | DNA/RNA | IV-C repeat |
| SEQ ID NO: 780 | DNA/RNA | IV-D repeat |
| SEQ ID NO: 781 | DNA/RNA | IV-D repeat |
| SEQ ID NO: 782 | DNA/RNA | IV-D repeat |
| SEQ ID NO: 783 | DNA/RNA | IV-D repeat |
| SEQ ID NO: 784 | DNA/RNA | IV-D repeat |
| SEQ ID NO: 785 | DNA/RNA | IV-D repeat |
| SEQ ID NO: 786 | DNA/RNA | IV-D repeat |
| SEQ ID NO: 787 | DNA/RNA | IV-D repeat |
| SEQ ID NO: 788 | DNA/RNA | IV-D repeat |
| SEQ ID NO: 789 | DNA/RNA | IV-D repeat |
| SEQ ID NO: 790 | DNA/RNA | IV-D repeat |
| SEQ ID NO: 791 | DNA/RNA | IV-D repeat |
| SEQ ID NO: 792 | DNA/RNA | IV-D repeat |
| SEQ ID NO: 793 | DNA/RNA | IV-D repeat |
| SEQ ID NO: 794 | DNA/RNA | IV-D repeat |
| SEQ ID NO: 795 | DNA/RNA | IV-D repeat |
| SEQ ID NO: 796 | DNA/RNA | IV-D repeat |
| SEQ ID NO: 797 | DNA/RNA | IV-D repeat |
| SEQ ID NO: 798 | DNA/RNA | IV-D repeat |
| SEQ ID NO: 799 | DNA/RNA | IV-D repeat |
| SEQ ID NO: 800 | DNA/RNA | IV-D repeat |
| SEQ ID NO: 801 | DNA/RNA | IV-D repeat |
| SEQ ID NO: 802 | DNA/RNA | IV-D repeat |
| SEQ ID NO: 803 | DNA/RNA | IV-D repeat |
| SEQ ID NO: 804 | DNA/RNA | IV-D repeat |
| SEQ ID NO: 805 | DNA/RNA | IV-E repeat |
| SEQ ID NO: 806 | DNA/RNA | IV-E repeat |
| SEQ ID NO: 807 | DNA/RNA | IV-E repeat |
| SEQ ID NO: 808 | DNA/RNA | IV-E repeat |
| SEQ ID NO: 809 | DNA/RNA | IV-E repeat |
| SEQ ID NO: 810 | DNA/RNA | IV-E repeat |
| SEQ ID NO: 811 | DNA/RNA | IV-E repeat |
| SEQ ID NO: 812 | DNA/RNA | IV-E repeat |
| SEQ ID NO: 813 | DNA/RNA | IV-E repeat |
| SEQ ID NO: 814 | DNA/RNA | IV-E repeat |
| SEQ ID NO: 815 | DNA/RNA | IV-E repeat |
| SEQ ID NO: 816 | DNA/RNA | IV-E repeat |
| SEQ ID NO: 817 | DNA/RNA | IV-E repeat |
| SEQ ID NO: 818 | DNA/RNA | IV-E repeat |
| SEQ ID NO: 819 | DNA/RNA | IV-E repeat |
| SEQ ID NO: 820 | DNA/RNA | IV-E repeat |
| SEQ ID NO: 821 | DNA/RNA | IV-E repeat |
| SEQ ID NO: 822 | DNA/RNA | IV-E repeat |
| SEQ ID NO: 823 | DNA/RNA | IV-E repeat |
| SEQ ID NO: 824 | DNA/RNA | IV-E repeat |
| SEQ ID NO: 825 | DNA/RNA | IV-E repeat |
| SEQ ID NO: 826 | DNA/RNA | IV-E repeat |
| SEQ ID NO: 827 | DNA/RNA | IV-E repeat |
| SEQ ID NO: 828 | DNA/RNA | IV-E repeat |
| SEQ ID NO: 829 | DNA/RNA | IV-E repeat |
| SEQ ID NO: 830 | DNA/RNA | V-A repeat |
| SEQ ID NO: 831 | DNA/RNA | V-A repeat |
| SEQ ID NO: 832 | DNA/RNA | V-A repeat |
| SEQ ID NO: 833 | DNA/RNA | V-A repeat |
| SEQ ID NO: 834 | DNA/RNA | V-A repeat |
| SEQ ID NO: 835 | DNA/RNA | V-A repeat |
| SEQ ID NO: 836 | DNA/RNA | V-A repeat |
| SEQ ID NO: 837 | DNA/RNA | V-A repeat |
| SEQ ID NO: 838 | DNA/RNA | V-A repeat |
| SEQ ID NO: 839 | DNA/RNA | V-A repeat |
| SEQ ID NO: 840 | DNA/RNA | V-A repeat |
| SEQ ID NO: 841 | DNA/RNA | V-A repeat |
| SEQ ID NO: 842 | DNA/RNA | V-A repeat |
| SEQ ID NO: 843 | DNA/RNA | V-A repeat |
| SEQ ID NO: 844 | DNA/RNA | V-A repeat |
| SEQ ID NO: 845 | DNA/RNA | V-A repeat |
| SEQ ID NO: 846 | DNA/RNA | V-A repeat |
| SEQ ID NO: 847 | DNA/RNA | V-A repeat |
| SEQ ID NO: 848 | DNA/RNA | V-A repeat |
| SEQ ID NO: 849 | DNA/RNA | V-A repeat |
| SEQ ID NO: 850 | DNA/RNA | V-A repeat |
| SEQ ID NO: 851 | DNA/RNA | V-A repeat |
| SEQ ID NO: 852 | DNA/RNA | V-A repeat |
| SEQ ID NO: 853 | DNA/RNA | V-A repeat |
| SEQ ID NO: 854 | DNA/RNA | V-A repeat |
| SEQ ID NO: 855 | DNA/RNA | V-A repeat |
| SEQ ID NO: 856 | DNA/RNA | V-A repeat |
| SEQ ID NO: 857 | DNA/RNA | V-A repeat |
| SEQ ID NO: 858 | DNA/RNA | V-B repeat |
| SEQ ID NO: 859 | DNA/RNA | V-B repeat |
| SEQ ID NO: 860 | DNA/RNA | V-D repeat |
| SEQ ID NO: 861 | DNA/RNA | V-D repeat |
| SEQ ID NO: 862 | DNA/RNA | V-E repeat |
| SEQ ID NO: 863 | DNA/RNA | V-E repeat |
| SEQ ID NO: 864 | DNA/RNA | V-F repeat |
| SEQ ID NO: 865 | DNA/RNA | V-F repeat |
| SEQ ID NO: 866 | DNA/RNA | V-F repeat |
| SEQ ID NO: 867 | DNA/RNA | V-F repeat |
| SEQ ID NO: 868 | DNA/RNA | V-F repeat |
| SEQ ID NO: 869 | DNA/RNA | V-F repeat |
| SEQ ID NO: 870 | DNA/RNA | V-F repeat |
| SEQ ID NO: 871 | DNA/RNA | V-F repeat |
| SEQ ID NO: 872 | DNA/RNA | V-F repeat |
| SEQ ID NO: 873 | DNA/RNA | V-F repeat |
| SEQ ID NO: 874 | DNA/RNA | V-F repeat |
| SEQ ID NO: 875 | DNA/RNA | V-F repeat |
| SEQ ID NO: 876 | DNA/RNA | V-F repeat |
| SEQ ID NO: 877 | DNA/RNA | V-F repeat |
| SEQ ID NO: 878 | DNA/RNA | V-F repeat |
| SEQ ID NO: 879 | DNA/RNA | V-F repeat |
| SEQ ID NO: 880 | DNA/RNA | V-F repeat |
| SEQ ID NO: 881 | DNA/RNA | V-F repeat |
| SEQ ID NO: 882 | DNA/RNA | V-F repeat |
| SEQ ID NO: 883 | DNA/RNA | V-F repeat |
| SEQ ID NO: 884 | DNA/RNA | V-F repeat |
| SEQ ID NO: 885 | DNA/RNA | V-G repeat |
| SEQ ID NO: 886 | DNA/RNA | V-I repeat |
| SEQ ID NO: 887 | DNA/RNA | V-I repeat |
| SEQ ID NO: 888 | DNA/RNA | V-J repeat |
| SEQ ID NO: 889 | DNA/RNA | V-J repeat |
| SEQ ID NO: 890 | DNA/RNA | V-J repeat |
| SEQ ID NO: 891 | DNA/RNA | V-J repeat |
| SEQ ID NO: 892 | DNA/RNA | V-J repeat |
| SEQ ID NO: 893 | DNA/RNA | V-J repeat |
| SEQ ID NO: 894 | DNA/RNA | V-J repeat |
| SEQ ID NO: 895 | DNA/RNA | V-J repeat |
| SEQ ID NO: 896 | DNA/RNA | V-K repeat |
| SEQ ID NO: 897 | DNA/RNA | V-K repeat |
| SEQ ID NO: 898 | DNA/RNA | V-K repeat |
| SEQ ID NO: 899 | DNA/RNA | V-K repeat |
| SEQ ID NO: 900 | DNA/RNA | V-K repeat |
| SEQ ID NO: 901 | DNA/RNA | VI-A repeat |
| SEQ ID NO: 902 | DNA/RNA | VI-A repeat |
| SEQ ID NO: 903 | DNA/RNA | VI-A repeat |
| SEQ ID NO: 904 | DNA/RNA | VI-A repeat |
| SEQ ID NO: 905 | DNA/RNA | VI-A repeat |
| SEQ ID NO: 906 | DNA/RNA | VI-A repeat |
| SEQ ID NO: 907 | DNA/RNA | VI-A repeat |
| SEQ ID NO: 908 | DNA/RNA | VI-A repeat |
| SEQ ID NO: 909 | DNA/RNA | VI-A repeat |
| SEQ ID NO: 910 | DNA/RNA | VI-A repeat |
| SEQ ID NO: 911 | DNA/RNA | VI-A repeat |
| SEQ ID NO: 912 | DNA/RNA | VI-A repeat |
| SEQ ID NO: 913 | DNA/RNA | VI-B repeat |
| SEQ ID NO: 914 | DNA/RNA | VI-B repeat |
| SEQ ID NO: 915 | DNA/RNA | VI-B repeat |
| SEQ ID NO: 916 | DNA/RNA | VI-B repeat |
| SEQ ID NO: 917 | DNA/RNA | VI-B repeat |
| SEQ ID NO: 918 | DNA/RNA | VI-B repeat |
| SEQ ID NO: 919 | DNA/RNA | VI-B repeat |
| SEQ ID NO: 920 | DNA/RNA | VI-B repeat |
| SEQ ID NO: 921 | DNA/RNA | VI-B repeat |
| SEQ ID NO: 922 | DNA/RNA | VI-B repeat |
| SEQ ID NO: 923 | DNA/RNA | VI-B repeat |
| SEQ ID NO: 924 | DNA/RNA | VI-B repeat |
| SEQ ID NO: 925 | DNA/RNA | VI-B repeat |
| SEQ ID NO: 926 | DNA/RNA | VI-C repeat |
| SEQ ID NO: 927 | DNA/RNA | VI-D repeat |
| SEQ ID NO: 928 | DNA/RNA | VI-D repeat |
| SEQ ID NO: 929 | DNA/RNA | VI-D repeat |
| SEQ ID NO: 930 | DNA/RNA | II-B tracrRNA |
| SEQ ID NO: 931 | DNA/RNA | II-B tracrRNA |
| SEQ ID NO: 932 | DNA/RNA | II-B tracrRNA |
| SEQ ID NO: 933 | DNA/RNA | II-B tracrRNA |
| SEQ ID NO: 934 | DNA/RNA | II-B tracrRNA |
| SEQ ID NO: 935 | DNA/RNA | II-B tracrRNA |
| SEQ ID NO: 936 | DNA/RNA | II-A tracrRNA |
| SEQ ID NO: 937 | DNA/RNA | II-A tracrRNA |
| SEQ ID NO: 938 | DNA/RNA | II-A tracrRNA |
| SEQ ID NO: 939 | DNA/RNA | II-A tracrRNA |
| SEQ ID NO: 940 | DNA/RNA | II-A tracrRNA |
| SEQ ID NO: 941 | DNA/RNA | II-A tracrRNA |
| SEQ ID NO: 942 | DNA/RNA | II-A tracrRNA |
| SEQ ID NO: 943 | DNA/RNA | II-A tracrRNA |
| SEQ ID NO: 944 | DNA/RNA | II-A tracrRNA |
| SEQ ID NO: 945 | DNA/RNA | II-A tracrRNA |
| SEQ ID NO: 946 | DNA/RNA | II-A tracrRNA |
| SEQ ID NO: 947 | DNA/RNA | II-A tracrRNA |
| SEQ ID NO: 948 | DNA/RNA | II-A tracrRNA |
| SEQ ID NO: 949 | DNA/RNA | II-A tracrRNA |
| SEQ ID NO: 950 | DNA/RNA | II-A tracrRNA |
| SEQ ID NO: 951 | DNA/RNA | II-A tracrRNA |
| SEQ ID NO: 952 | DNA/RNA | II-A tracrRNA |
| SEQ ID NO: 953 | DNA/RNA | II-A tracrRNA |
| SEQ ID NO: 954 | DNA/RNA | II-A tracrRNA |
| SEQ ID NO: 955 | DNA/RNA | II-A tracrRNA |
| SEQ ID NO: 956 | DNA/RNA | II-A tracrRNA |
| SEQ ID NO: 957 | DNA/RNA | II-A tracrRNA |
| SEQ ID NO: 958 | DNA/RNA | II-A tracrRNA |
| SEQ ID NO: 959 | DNA/RNA | II-A tracrRNA |
| SEQ ID NO: 960 | DNA/RNA | II-A tracrRNA |
| SEQ ID NO: 961 | DNA/RNA | II-A tracrRNA |
| SEQ ID NO: 962 | DNA/RNA | II-A tracrRNA |
| SEQ ID NO: 963 | DNA/RNA | II-A tracrRNA |
| SEQ ID NO: 964 | DNA/RNA | II-A tracrRNA |
| SEQ ID NO: 965 | DNA/RNA | II-C tracrRNA |
| SEQ ID NO: 966 | DNA/RNA | II-C tracrRNA |
| SEQ ID NO: 967 | DNA/RNA | II-C tracrRNA |
| SEQ ID NO: 968 | DNA/RNA | II-C tracrRNA |
| SEQ ID NO: 969 | DNA/RNA | II-C tracrRNA |
| SEQ ID NO: 970 | DNA/RNA | II-C tracrRNA |
| SEQ ID NO: 971 | DNA/RNA | II-C tracrRNA |
| SEQ ID NO: 972 | DNA/RNA | II-C tracrRNA |
| SEQ ID NO: 973 | DNA/RNA | II-C tracrRNA |
| SEQ ID NO: 974 | DNA/RNA | II-C tracrRNA |
| SEQ ID NO: 975 | DNA/RNA | II-C tracrRNA |
| SEQ ID NO: 976 | DNA/RNA | II-C tracrRNA |
| SEQ ID NO: 977 | DNA/RNA | II-C tracrRNA |
| SEQ ID NO: 978 | DNA/RNA | II-C tracrRNA |
| SEQ ID NO: 979 | DNA/RNA | II-C tracrRNA |
| SEQ ID NO: 980 | DNA/RNA | II-C tracrRNA |
| SEQ ID NO: 981 | DNA/RNA | II-C tracrRNA |
| SEQ ID NO: 982 | DNA/RNA | II-C tracrRNA |
| SEQ ID NO: 983 | DNA/RNA | II-C tracrRNA |
| SEQ ID NO: 984 | DNA/RNA | II-C tracrRNA |
| SEQ ID NO: 985 | DNA/RNA | II-C tracrRNA |
| SEQ ID NO: 986 | DNA/RNA | II-C tracrRNA |
| SEQ ID NO: 987 | DNA/RNA | II-C tracrRNA |
| SEQ ID NO: 988 | DNA/RNA | II-C tracrRNA |
| SEQ ID NO: 989 | DNA/RNA | II-C tracrRNA |
| SEQ ID NO: 990 | DNA/RNA | II-C tracrRNA |
| SEQ ID NO: 991 | DNA/RNA | II-C tracrRNA |
| SEQ ID NO: 992 | DNA/RNA | II-C tracrRNA |
| SEQ ID NO: 993 | DNA/RNA | V-B tracrRNA |
| SEQ ID NO: 994 | DNA/RNA | V-B tracrRNA |
| SEQ ID NO: 995 | DNA/RNA | V-B tracrRNA |
| SEQ ID NO: 996 | DNA/RNA | V-B tracrRNA |
| SEQ ID NO: 997 | DNA/RNA | U-K tracrRNA |
| SEQ ID NO: 998 | DNA/RNA | U-K tracrRNA |
| SEQ ID NO: 999 | DNA/RNA | V-E tracrRNA |
| SEQ ID NO: 1000 | DNA/RNA | V-E tracrRNA |
| SEQ ID NO: 1001 | DNA/RNA | II-A tracrRNA |
| SEQ ID NO: 1002 | DNA/RNA | II-A tracrRNA |
| SEQ ID NO: 1003 | DNA/RNA | II-A tracrRNA |
| SEQ ID NO: 1004 | DNA/RNA | II-A tracrRNA |
| SEQ ID NO: 1005 | DNA/RNA | II-A tracrRNA |
| SEQ ID NO: 1006 | DNA/RNA | II-A tracrRNA |
| SEQ ID NO: 1007 | DNA/RNA | II-A tracrRNA |
| SEQ ID NO: 1008 | DNA/RNA | II-A tracrRNA |
| SEQ ID NO: 1009 | DNA/RNA | II-A tracrRNA |
| SEQ ID NO: 1010 | DNA/RNA | II-A tracrRNA |
| SEQ ID NO: 1011 | DNA/RNA | II-A tracrRNA |
| SEQ ID NO: 1012 | DNA/RNA | II-A tracrRNA |
| SEQ ID NO: 1013 | DNA/RNA | II-A tracrRNA |
| SEQ ID NO: 1014 | DNA/RNA | II-A tracrRNA |
| SEQ ID NO: 1015 | DNA/RNA | II-A tracrRNA |
| SEQ ID NO: 1016 | DNA/RNA | II-A tracrRNA |
| SEQ ID NO: 1017 | DNA/RNA | II-A tracrRNA |
| SEQ ID NO: 1018 | DNA/RNA | II-A tracrRNA |
| SEQ ID NO: 1019 | DNA/RNA | II-A tracrRNA |
| SEQ ID NO: 1020 | DNA/RNA | II-A tracrRNA |
| SEQ ID NO: 1021 | DNA/RNA | II-A tracrRNA |
| SEQ ID NO: 1022 | DNA/RNA | II-A tracrRNA |
| SEQ ID NO: 1023 | DNA/RNA | II-A tracrRNA |
| SEQ ID NO: 1024 | DNA/RNA | II-A tracrRNA |
| SEQ ID NO: 1025 | DNA/RNA | II-A tracrRNA |
| SEQ ID NO: 1026 | DNA/RNA | II-A tracrRNA |
| SEQ ID NO: 1027 | DNA/RNA | II-A tracrRNA |
| SEQ ID NO: 1028 | DNA/RNA | II-A tracrRNA |
| SEQ ID NO: 1029 | DNA/RNA | II-A tracrRNA |
| SEQ ID NO: 1030 | DNA/RNA | II-A tracrRNA |
| SEQ ID NO: 1031 | DNA/RNA | II-A tracrRNA |
| SEQ ID NO: 1032 | DNA/RNA | II-A tracrRNA |
| SEQ ID NO: 1033 | DNA/RNA | II-A tracrRNA |
| SEQ ID NO: 1034 | DNA/RNA | II-A tracrRNA |
| SEQ ID NO: 1035 | DNA/RNA | II-A tracrRNA |
| SEQ ID NO: 1036 | DNA/RNA | II-A tracrRNA |
| SEQ ID NO: 1037 | DNA/RNA | II-A tracrRNA |
| SEQ ID NO: 1038 | DNA/RNA | II-A tracrRNA |
| SEQ ID NO: 1039 | DNA/RNA | II-A tracrRNA |
| SEQ ID NO: 1040 | DNA/RNA | II-A tracrRNA |
| SEQ ID NO: 1041 | DNA/RNA | II-A tracrRNA |
| SEQ ID NO: 1042 | DNA/RNA | II-A tracrRNA |
| SEQ ID NO: 1043 | DNA/RNA | II-A tracrRNA |
| SEQ ID NO: 1044 | DNA/RNA | II-A tracrRNA |
| SEQ ID NO: 1045 | DNA/RNA | II-A tracrRNA |
| SEQ ID NO: 1046 | DNA/RNA | II-A tracrRNA |
| SEQ ID NO: 1047 | DNA/RNA | II-A tracrRNA |
| SEQ ID NO: 1048 | DNA/RNA | II-A tracrRNA |
| SEQ ID NO: 1049 | DNA/RNA | II-A tracrRNA |
| SEQ ID NO: 1050 | DNA/RNA | II-A tracrRNA |
| SEQ ID NO: 1051 | DNA/RNA | II-A tracrRNA |
| SEQ ID NO: 1052 | DNA/RNA | II-A tracrRNA |
| SEQ ID NO: 1053 | DNA/RNA | II-A tracrRNA |
| SEQ ID NO: 1054 | DNA/RNA | II-A tracrRNA |
| SEQ ID NO: 1055 | DNA/RNA | II-A tracrRNA |
| SEQ ID NO: 1056 | DNA/RNA | II-A tracrRNA |
| SEQ ID NO: 1057 | DNA/RNA | II-A tracrRNA |
| SEQ ID NO: 1058 | DNA/RNA | II-A tracrRNA |
| SEQ ID NO: 1059 | DNA/RNA | II-A tracrRNA |
| SEQ ID NO: 1060 | DNA/RNA | II-A tracrRNA |
| SEQ ID NO: 1061 | DNA/RNA | II-A tracrRNA |
| SEQ ID NO: 1062 | DNA/RNA | II-A tracrRNA |
| SEQ ID NO: 1063 | DNA/RNA | II-A tracrRNA |
| SEQ ID NO: 1064 | DNA/RNA | II-A tracrRNA |
| SEQ ID NO: 1065 | DNA/RNA | II-A tracrRNA |
| SEQ ID NO: 1066 | DNA/RNA | II-A tracrRNA |
| SEQ ID NO: 1067 | DNA/RNA | II-A tracrRNA |
| SEQ ID NO: 1068 | DNA/RNA | II-A tracrRNA |
| SEQ ID NO: 1069 | DNA/RNA | II-A tracrRNA |
| SEQ ID NO: 1070 | DNA/RNA | II-A tracrRNA |
| SEQ ID NO: 1071 | DNA/RNA | II-A tracrRNA |
| SEQ ID NO: 1072 | DNA/RNA | II-A tracrRNA |
| SEQ ID NO: 1073 | DNA/RNA | II-A tracrRNA |
| SEQ ID NO: 1074 | DNA/RNA | II-A tracrRNA |
| SEQ ID NO: 1075 | DNA/RNA | II-A tracrRNA |
| SEQ ID NO: 1076 | DNA/RNA | II-A tracrRNA |
| SEQ ID NO: 1077 | DNA/RNA | II-A tracrRNA |
| SEQ ID NO: 1078 | DNA/RNA | II-A tracrRNA |
| SEQ ID NO: 1079 | DNA/RNA | II-A tracrRNA |
| SEQ ID NO: 1080 | DNA/RNA | II-A tracrRNA |
| SEQ ID NO: 1081 | DNA/RNA | II-A tracrRNA |
| SEQ ID NO: 1082 | DNA/RNA | II-A tracrRNA |
| SEQ ID NO: 1083 | DNA/RNA | II-A tracrRNA |
| SEQ ID NO: 1084 | DNA/RNA | II-A tracrRNA |
| SEQ ID NO: 1085 | DNA/RNA | II-A tracrRNA |
| SEQ ID NO: 1086 | DNA/RNA | II-A tracrRNA |
| SEQ ID NO: 1087 | DNA/RNA | II-A tracrRNA |
| SEQ ID NO: 1088 | DNA/RNA | II-A tracrRNA |
| SEQ ID NO: 1089 | DNA/RNA | II-A tracrRNA |
| SEQ ID NO: 1090 | DNA/RNA | II-A tracrRNA |
| SEQ ID NO: 1091 | DNA/RNA | II-A tracrRNA |
| SEQ ID NO: 1092 | DNA/RNA | II-A tracrRNA |
| SEQ ID NO: 1093 | DNA/RNA | II-A tracrRNA |
| SEQ ID NO: 1094 | DNA/RNA | II-A tracrRNA |
| SEQ ID NO: 1095 | DNA/RNA | II-A tracrRNA |
| SEQ ID NO: 1096 | DNA/RNA | II-A tracrRNA |
| SEQ ID NO: 1097 | DNA/RNA | II-A tracrRNA |
| SEQ ID NO: 1098 | DNA/RNA | II-A tracrRNA |
| SEQ ID NO: 1099 | DNA/RNA | II-A tracrRNA |
| SEQ ID NO: 1100 | DNA/RNA | II-A tracrRNA |
| SEQ ID NO: 1101 | DNA/RNA | II-A tracrRNA |
| SEQ ID NO: 1102 | DNA/RNA | II-A tracrRNA |
| SEQ ID NO: 1103 | DNA/RNA | II-A tracrRNA |
| SEQ ID NO: 1104 | DNA/RNA | II-A tracrRNA |
| SEQ ID NO: 1105 | DNA/RNA | II-A tracrRNA |
| SEQ ID NO: 1106 | DNA/RNA | II-A tracrRNA |
| SEQ ID NO: 1107 | DNA/RNA | II-A tracrRNA |
| SEQ ID NO: 1108 | DNA/RNA | II-A tracrRNA |
| SEQ ID NO: 1109 | DNA/RNA | II-A tracrRNA |
| SEQ ID NO: 1110 | DNA/RNA | II-A tracrRNA |
| SEQ ID NO: 1111 | DNA/RNA | II-A tracrRNA |
| SEQ ID NO: 1112 | DNA/RNA | II-A tracrRNA |
| SEQ ID NO: 1113 | DNA/RNA | II-A tracrRNA |
| SEQ ID NO: 1114 | DNA/RNA | II-A tracrRNA |
| SEQ ID NO: 1115 | DNA/RNA | II-A tracrRNA |
| SEQ ID NO: 1116 | DNA/RNA | II-A tracrRNA |
| SEQ ID NO: 1117 | DNA/RNA | II-A tracrRNA |
| SEQ ID NO: 1118 | DNA/RNA | II-A tracrRNA |
| SEQ ID NO: 1119 | DNA/RNA | II-A tracrRNA |
| SEQ ID NO: 1120 | DNA/RNA | II-A tracrRNA |
| SEQ ID NO: 1121 | DNA/RNA | II-A tracrRNA |
| SEQ ID NO: 1122 | DNA/RNA | II-A tracrRNA |
| SEQ ID NO: 1123 | DNA/RNA | II-A tracrRNA |
| SEQ ID NO: 1124 | DNA/RNA | II-A tracrRNA |
| SEQ ID NO: 1125 | DNA/RNA | II-A tracrRNA |
| SEQ ID NO: 1126 | DNA/RNA | II-A tracrRNA |
| SEQ ID NO: 1127 | DNA/RNA | II-A tracrRNA |
| SEQ ID NO: 1128 | DNA/RNA | II-A tracrRNA |
| SEQ ID NO: 1129 | DNA/RNA | II-A tracrRNA |
| SEQ ID NO: 1130 | DNA/RNA | II-A tracrRNA |
| SEQ ID NO: 1131 | DNA/RNA | II-A tracrRNA |
| SEQ ID NO: 1132 | DNA/RNA | II-A tracrRNA |
| SEQ ID NO: 1133 | DNA/RNA | II-A tracrRNA |
| SEQ ID NO: 1134 | DNA/RNA | II-A tracrRNA |
| SEQ ID NO: 1135 | DNA/RNA | II-A tracrRNA |
| SEQ ID NO: 1136 | DNA/RNA | II-A tracrRNA |
| SEQ ID NO: 1137 | DNA/RNA | II-A tracrRNA |
| SEQ ID NO: 1138 | DNA/RNA | II-A tracrRNA |
| SEQ ID NO: 1139 | DNA/RNA | II-A tracrRNA |
| SEQ ID NO: 1140 | DNA/RNA | II-A tracrRNA |
| SEQ ID NO: 1141 | DNA/RNA | II-A tracrRNA |
| SEQ ID NO: 1142 | DNA/RNA | II-A tracrRNA |
| SEQ ID NO: 1143 | DNA/RNA | II-A tracrRNA |
| SEQ ID NO: 1144 | DNA/RNA | II-A tracrRNA |
| SEQ ID NO: 1145 | DNA/RNA | II-A tracrRNA |
| SEQ ID NO: 1146 | Protein/1 | Cas1 3GOD |
| SEQ ID NO: 1147 | Protein/1 | Cas1 4XTK |
| SEQ ID NO: 1148 | Protein/1 | Cas1 2YZS |
| SEQ ID NO: 1149 | Protein/1 | Cas2 2IVY |
| SEQ ID NO: 1150 | Protein/1 | Cas2 2I8E |
| SEQ ID NO: 1151 | Protein/1 | Cas2 3EXC |
| SEQ ID NO: 1152 | Protein/1 | Cas2 4P6I-1 |
| SEQ ID NO: 1153 | Protein/1 | Cas2 4P6I-2 |
| SEQ ID NO: 1154 | Protein/1 | Cas3 4QQW-1 |
| SEQ ID NO: 1155 | Protein/1 | Cas3 A 4QQX-1 |
| SEQ ID NO: 1156 | Protein/1 | Cas3 A 4QQZ-1 |
| SEQ ID NO: 1157 | Protein/1 | Cas3 A 4QQY-1 |
| SEQ ID NO: 1158 | Protein/1 | Cas3 B 3S4L |
| SEQ ID NO: 1159 | Protein/1 | Cas3 B 3SKD |
| SEQ ID NO: 1160 | Protein/1 | Cas4 4IC1 |
| SEQ ID NO: 1161 | Protein/1 | Cas5 3KG4 |
| SEQ ID NO: 1162 | Protein/1 | Cas5 3VZI |
| SEQ ID NO: 1163 | Protein/1 | Cas5 3VZH |
| SEQ ID NO: 1164 | Protein/1 | Cas6 2XLI-1 |
| SEQ ID NO: 1165 | Protein/1 | Cas6 1WJ9 |
| SEQ ID NO: 1166 | Protein/1 | Cas6 3I4H |
| SEQ ID NO: 1167 | Protein/1 | Cas7 3PS0 |
| SEQ ID NO: 1168 | Protein/1 | Cas7 4N0L |
| SEQ ID NO: 1169 | Protein/1 | Cas8 4AN8 |
| SEQ ID NO: 1170 | Protein/1 | Cas9 4OGC |
| SEQ ID NO: 1171 | Protein/1 | Cas9 4OO8 |
| SEQ ID NO: 1172 | Protein/1 | Cas9 4CMP |
| SEQ ID NO: 1173 | Protein/1 | Cas10 3UNG |
| SEQ ID NO: 1174 | Protein/1 | Cas10 4DOZ |
| SEQ ID NO: 1175 | Protein/1 | Cas11 2ZCA |
| SEQ ID NO: 1176 | Protein/1 | Cas11 2ZOP |
| SEQ ID NO: 1177 | Protein/1 | Cas11 2OEB |
| SEQ ID NO: 1178 | Protein/1 | Cas12 5NFV |
| SEQ ID NO: 1179 | Protein/1 | Cas12 5WQE |
| SEQ ID NO: 1180 | Protein/1 | Cas12 6NY1 |
| SEQ ID NO: 1181 | Protein/1 | Cas13 5XWP |
| SEQ ID NO: 1182 | Protein/1 | Cas13 5W1I |
| SEQ ID NO: 1183 | Protein/1 | Cas13 6DTD |
| SEQ ID NO: 1184 | Protein/1 | Cas13 6AAY |
| SEQ ID NO: 1185 | DNA/RNA | Prs54 V-A acrRNA |
| SEQ ID NO: 1186 | DNA/RNA | Prs55 V-A acrRNA |
| SEQ ID NO: 1187 | DNA/RNA | Prs65 I-E acrRNA |
| SEQ ID NO: 1188 | DNA/RNA | Prs54 V-A acrRNA |
| SEQ ID NO: 1189 | DNA/RNA | Prs81 V-A acrRNA |
| SEQ ID NO: 1190 | DNA/RNA | Prs108 I-C acrRNA |
| SEQ ID NO: 1191 | DNA/RNA | prs109 I-C acrRNA |
| SEQ ID NO: 1192 | DNA/RNA | Prs120 I-C acrRNA |
| SEQ ID NO: 1193 | DNA/RNA | Prs121 I-C acrRNA |
| SEQ ID NO: 1194 | DNA/RNA | Ors37 I-E acrRNA |
| SEQ ID NO: 1195 | DNA/RNA | Ors38a I-E acrRNA |
| SEQ ID NO: 1196 | DNA/RNA | Fragment comprising V-A |
| | | acrRNAVA1 |
| SEQ ID NO: 1197 | DNA/RNA | Fragment comprising V-A |
| | | acrRNAVA2 |
| SEQ ID NO: 1198 | DNA/RNA | Fragment comprising V-A |
| | | acrRNAVA3 |
| SEQ ID NO: 1199 | DNA/RNA | Fragment comprising acrRNAIE1 |
| SEQ ID NO: 1200 | DNA/RNA | Fragment comprising acrRNAIC1 |
| SEQ ID NO: 1201 | DNA/RNA | AcrRNAIE1 |
| SEQ ID NO: 1202 | DNA/RNA | AcrRNAIE2 |
| SEQ ID NO: 1203 | DNA/RNA | AcrRNAIC1 |
| SEQ ID NO: 1204 | DNA/RNA | AcrRNAVA1 |
| SEQ ID NO: 1205 | DNA/RNA | AcrRNAVA2 |
| SEQ ID NO: 1206 | DNA/RNA | AcrRNAVA3 |
| SEQ ID NO: 1207 | DNA/RNA | Frs acrRNA 773 |
| SEQ ID NO: 1208 | DNA/RNA | Frs acrRNA 865 |
| SEQ ID NO: 1209 | DNA/RNA | Frs acrRNA 1792 |
| SEQ ID NO: 1210 | DNA/RNA | Frs acrRNA 1794 |
| SEQ ID NO: 1211 | DNA/RNA | native I-F PA14 |
| SEQ ID NO: 1212 | DNA/RNA | native V-A repeat |
| SEQ ID NO: 1213 | DNA/RNA | Synthetic acrRNA 865 |

### REFERENCES

Cady, K. C. et al. (2012) ‘The CRISPR/Cas adaptive immune system of Pseudomonas aeruginosa mediates resistance to naturally occurring and engineered phages’, Journal of bacteriology, 194(21), pp. 5728-5738.
Marino, N. D. et al. Discovery of widespread type I and type V CRISPR-Cas inhibitors. Science 362, 240-242 (2018).

### EXAMPLES

### Materials and Methods

Chemicals used in the examples herein, e.g. for buffers and substrates, are commercial products of at least reagent grade. Water utilized in the examples was de-ionized, MilliQ water.

TABLE 1
Chemicals used

| Compound | Abbr. | Use |
|---|---|---|
| Sucrose | | Used in the preparation of competent cells of PA14 |
| | | and PAO1. This special treatment allows these cells |
| | | to be in a state of competency, that is, permitting |
| | | the uptake of DNA from their immediate |
| | | surroundings (transformation). |
| L-Arabinose | L-ara | For induction of gene expression under pBad |
| | | promoter |
| Isopropyl β- d-1- | IPTG | For induction of gene expression under ptac |
| thiogalactopyranoside | | promoter |
| Magnesium sulfate | MgSO4 | For supplementation of solid LB-media used for |
| | | phage-related assays |
| Sodium Chloride | NaCl | For buffer solutions |
| Calcium chloride | CaCl2 | Used in the preparation of competent cells of |
| | | E. coli. This special treatment allows these cells to |
| | | be in a state of competency, permitting the pick up |
| | | DNA through transformation at higher efficiencies. |
| Gentamycin sulfate | Genta | For antibiotic selection of strains carrying |
| | | pHerd30T and/or variants |
| Carbenicillin | Carb | For antibiotic selection of strains carrying |
| | | pMMbHE67 and/or variants |
| Hydrochloric acid | HCl | For buffer solutions |
| Agar | | For supplementation of solid growth medium |
| Lysogenic broth | LB | For growth media |
| Deionized Water | ddH2O | Solvent for antibiotics and buffer/media |
| Tris-base | | For SM-Buffer |
| Chloroform | | For phage propagation |

TABLE 2
Medias and buffers

| Name | Composition |
|---|---|
| SM-Buffer | 5.8 g NaCl; 2 g MgSo4*7H2O; 50 mL Tris-Cl (1M, |
| | pH 7.5); volume adjusted to 1 liter with ddH2O; |
| | autoclaved |
| Tris-Cl | 121.1 g Tris base dissolved in 800 mL ddH2O; pH |
| | adjusted to 7.5 by adding concentrated HCl |
| Liquid bacterial | 8 g LB; 400 mL ddH2O; if needed supplemented |
| growth media | with antibiotics and inducers |
| Solid bacterial | 8 g LB; 6 g Agar; 400 mL ddH2O; if needed |
| growth media | supplemented with antibiotics and/or inducers |

TABLE 3
Antibiotic and inducer solutions

| | Stock | | Conc. in growth media | Conc. in growth media | |
|---|---|---|---|---|---|
| | conc. | | for E. coli | for Pseudomonas | |
| Carb | 50 | mg/mL | 100 ug/mL | 100 | ug/mL |
| Genta | 10 | mg/mL | 15 ug/mL | 50 | ug/mL |
| L-ara | 10% | w/v | — | 0.3% | w/v |

| | | | | |
|---|---|---|---|---|
| IPTG | 1M | — | 1 | mM |

The following microbial strains were prepared for use in the examples

TABLE 4
Cells

| Alias | Bacterium | Strain | genotype | Description | Reference |
|---|---|---|---|---|---|
| | E. coli | genehogs | | Used for enrichment of | Thermo Fisher |
| | | | | vector DNA and assembly of | catalog nos. C800- |
| | | | | constructs | 05 |
| RPR | P. aeroguinosa | PA14 | WT; I-F WT | PA14 harboring the native I- | Laboratory of |
| 145 | | | | F CRISPR-Cas system, | George O'Toole |
| | | | | targeting phage DMSm | NCBI database: |
| | | | | | NC_008463.1 |
| RPR | P. aeroguinosa | PA14 | I-F | PA14 harboring the native I- | Laboratory of |
| 146 | | | ΔCRISPR1 | F CRISPR-Cas system, lacking | George O'Toole |
| | | | | the CRISPR array which | Cady et al., 2012 |
| | | | | includes the spacer targeting | |
| | | | | phage DMSm | |
| RPR | P. aeruginosa | PAO1 | tn7::mbCpf1, | PAO1 harboring MbCpf1 and | Bondy-Denomy |
| 212 | | | ctx2::crRNA23 | a crRNA targeting phage | Lab |
| | | | | JBD30 | Marino et al., |
| | | | | | 2018 |
| RPR | P. aeruginosa | PAO1 | tn7::mbCpf1, | PAO1 harboring MbCpf1, | Bondy-Denomy |
| 213 | | | ctx2:: no | lacking the crRNA targeting | Lab |
| | | | crRNA23 | phage JBD30 | Marino et al., |
| | | | | | 2018 |
| SC | P. aeruginosa | PAscm4386 | with I-E | PA scm 4386 harboring a | Laboratory of |
| 115 | | | CRISPR-Cas | native I-E CRISPR-Cas system | George O'Toole |
| | | | | targeting phage JBD30 | NCBI: |
| | | | | | LOQZ00000000 |
| SC | P. aeruginosa | PAscm4386 | delta Cas3 | PA scm 4386 harboring a | Laboratory of |
| 116 | | | I-E CRISPR- | native I-E CRISPR-Cas system | George O'Toole |
| | | | Cas | Cas 3 knockout mutant | Cady et al., 2012 |
| RPR | P. aeruginosa | PAO1 | I-C CRISPR- | PAO1 harboring a | Bondy-Denomy |
| 148 | | | Cas; LL77 | heterologous I-C LL7 CRISPR- | Lab |
| | | | | Cas system and a crRNA | Marino et al., |
| | | | | targeting phage JBD30 | 2018 |
| RPR | P. aeruginosa | PAO1 | WT | PAO1 wild type (no crispr | Bondy-Denomy |
| 147 | | | | cas) | Lab |

The following phages were prepared for use in the examples

TABLE 5
Phages

| Name | Description | Reference |
|---|---|---|
| DMS3m | Pseudomonas phage capable of infecting | Laboratory of |
| | PA14 carrying a protospacer that is | George O'Toole |
| | targeted by the PA14 I-F CRISPR-Cas | NCBI database |
| | system | (DMS3): |
| | | NC_008717.1 |
| JBD30 | Pseudomonas phage capable of infecting | Bondy-Denomy |
| | PAO1 carrying a protospacer that is | Lab |
| | targeted by the PA scm4386 I-E CRISPR- | NCBI database: |
| | Cas system. | NC_020298.1 |

TABLE 6
Primers and oligonucleotides

| Name | Sequence 5′→3′ | Description |
|---|---|---|
| Prs1 | AAATTATUTCTAGCCCAAAAAAACGG | pHerd30t backbone amplification |
| Prs2 | ACTGGCCGUCGTITTACAACGTCG | pHerd30t backbone amplification |
| Prs3 | AGATTAGCGGATCCTACCTG | sequencing of repeat/acrRNA site |
| Prs4 | GCTGCAAGGCGATTAAGTTGG | sequencing of repeat/acrRNA site |
| Prs9 | ACGGCCAGUTGATACGATTAGGACAATGGTCACCGACG | amplification of acrRNAs ordered from |
| | | twist bioscience for insertion into |
| | | pHerd30T |
| Scp14 | AGTCCGAUCCCAACTATTTTGTCCGCCCAC | amplification of acrRNAs ordered from |
| | | twist bioscience for insertion into |
| | | pHerd30T |
| Prs31 | AATAATTUTCCGGGGCCTGCTCTC | amplification of acrRNA865 without |
| | | predicted native promoter for insertion |
| | | behind pBad |
| PRS40 | AATAATTUGGAGTATATATGCAACTACATAACGCC | amplification of acrRNA1792 without |
| | | predicted native promoter for insertion |
| | | behind pBad |
| PRS41 | AATAATTUATTGCAGGTAAGATGGCATCTATG | amplification of acrRNA1794 without |
| | | predicted native promoter for insertion |
| | | behind pBad |
| Ors15 | GTTCACTGCCGTATAGGCAGCTAAGAAAAACGGCCGACGCTT | full repeat I-F |
| Ors16 | CGGCCGTTTTTCTTAGCTGCCTATACGGCAGTGAACAAATTATT | full repeat I-F |
| Ors23 | TCTCGTTCACTGCCGGATAGGCAGCCAAGGAAATC | ″Synthetic″ acrRNA865 (palindromic |
| | | repeat only) |
| Ors24 | CAGTGATTTCCTTGGCTGCCTATCCGGCAGTGAAC | ″Synthetic″ acrRNA865 (palindromic |
| | | repeat only) |
| Prs54 | actgtttcuccatTGTTTTTAAACCATGTCAATTG | Cloning V-A acrRNA |
| Prs55 | actgtttcuccatTGGTCGCATCACAGCAAATAG | Cloning V-A acrRNA |
| Prs65 | actgtttcuccatCCGTGTTCCCCGCGTGTGC | Cloning I-E acrRNA |
| Prs54 | actgtttcuccatTGTTTTTAAACCATGTCAATTG | Cloning V-A acrRNA |
| Prs81 | acggccagUATAACCAATAAGCCGCTGATAATCCC | Cloning V-A acrRNA |
| Prs108 | acccaugagcaccatcatcgaccaggac | Cloning I-C acrRNA |
| prs109 | atggguatgtatatctccttcttaaagttaaac | Cloning I-C acrRNA |
| Prs120 | accgccgUggcgttagtcgattt | Cloning I-C acrRNA |
| Prs121 | acggcggUcaacctcatggacg | Cloning I-C acrRNA |
| Ors37 | GTGTTCCCCACGGGTGTGGGGATGAACCactggccgtt | Cloning acrRNA I-E |
| Ors38a | GGTTCATCCCCACACCCGTGGGGAACACatggagaaacagt | Cloning acrRNA I-E |

TABLE 7
DNA Fragments comprising acrRNA's
Active moieties in bold; primer sequences underlined and do not form part of the original
sequence

| Name | Sequence 5′→3′ | Description |
|---|---|---|
| Frs | AGTCCGATCCCAACTATTTTGTCCGCCCACTGGTGTTGCTGGACTACCTGT | DNA Fragment for amplification |
| acrRNA773 | CCAACATCTTCCGGGTTGGCGGTGAAGACCAGTTATCCAAGTACCGGAA | of I-F acrRNA773 |
| | GAATATCGGTCAGGCATAGGGACTAGCTCCAATGGAATGAGCCGCCAGG | sequence from NCBI database: |
| | ACGGCAAACCGAAGCCCCGCCATCGTGCGGGGCTTCTTTTTTTCCAGTCA | CP011110.1 |
| | GCAAACTTGAAATACACCCTTAAGGGTGTATTATTGTTTCCACGGAAGGC | |
| | GAATCGCCAACCCCTCACTGCCGTATAGGCAGCCCAGAATGTACGGAGA | |
| | TCACCACACGTCGGTGACCATTGTCCTAATCGTATCAACTGGCCGT | |
| Frs | AGTCCGATCCCAACTATTTTGTCCGCCCACGAGACAAGGTCGCCTTGTCT | DNA Fragment for amplification |
| acrRNA865 | CGACAACGCCCCCGCGAAAACCGCCCCTGTGAGCCGTCCGCCACCAGAC | of I-F acrRNA865 |
| | CCGCCACCGTCCTACCGCCCAAACCGCCCCGCGCGCGTCTACCCGCGTTA | sequence from NCBI database: |
| | GACCCGCGTTAGATTCGATTTCCGGGGCCTGCTCTCGCCGCCCCCTTCGG | NC_018012.1 |
| | CTTCGCTCAGGGCAGGCTTGCGACCGTCGCCCGATTGCGCGACAGTAGA | |
| | GACAGGGCGCCGCCTTGTCTGATGCTCTCGTTCACTGCCGGATAGGCAG | |
| | CCAAGGAAATCATGCTCGGCCACGGACGGTCTTCCCCCTGCGCCCCCGC | |
| | AACCTATTGTGCACATCGCCCGCCGTCGATCATGGCGGCCGTCGGTGACC | |
| | ATTGTCCTAATCGTATCAACTGGCCGT | |
| Frs | AGTCCGATCCCAACTATTTTGTCCGCCCACCACAGCCCTTAGCATTTCCAT | DNA Fragment for amplification |
| acrRNA1792 | GCTATACAAGTCGCACCTTAAGCAGTCTGGCAAACTGCCTTTTGGGATTA | of V-A acrRNA1792 |
| | CCAGTCCCAAACCTTGAACGCCTCTAAGGATTTAAAACCTTAGGGGTGTT | sequence from NCBI database: |
| | TATTTTATCTGGGAGTATATATGCAACTACATAACGCCATATTTGTTGATG | CP011377.1 |
| | GTCGCATCACAGCAAATAGCAGTCTAACGACCTTTTAAATTTCTACTGTT | |
| | TGTAGATTTCCTGTTGGGGATTATCAGCGGCTTATTGGTTATTTGCGCTTT | |
| | GACAGATTTTGTGTGAAATATCCTGACAGCAAGGCGGATTTTAACTGCTT | |
| | TGAGCCGTCGGTGACCATTGTCCTAATCGTATCAACTGGCCGT | |
| Frs | AGTCCGATCCCAACTATTTTGTCCGCCCACATGCTACATCGTTATGCATCA | DNA Fragment for amplification |
| acrRNA1794 | GTATTGATGGATGCAGGTTTTAGCTTTGAAAGTATTAAAGAGAAAACTAT | of V-A acrRNA1794 |
| | TGCTTTAAATAATAAGTTAATAGATAAACTTGATGAACTGGAGTTAGCTA | sequence from NCBI database: |
| | ATACAATATTCCATACCATTGCAGGTAAGATGGCATCTATGGGCAGAATG | CP011376.1 |
| | TAGCTTTTAACCATAACATCGCATTCTGCGGTGTTATGGTTTTGTTTTTAA | |
| | ACCATGTCAATTGTCTAACAACTTTTTAAATTTCTACTGTTTGTAGATTAG | |
| | ATTAGATAACCATGTCAATTAATCATAGGAGACTTTATGAGTCAAGTTAA | |
| | TGACCATTTAGTACTTATCTGTGGTGAATCTAGTACAGGTAAATCAGCGT | |
| | CGGTGACCATTGTCCTAATCGTATCAACTGGCCGT | |
| AcrRNAVA1 | ACTGTTTCTCCATTTTTTTAAAATGGTGAAAGTCTAACGACTATTTAAATT | DNA Fragment for amplification |
| | TCTACTATTTGTAGATACGAATTCAAGGTATTGGCGAAAATCTTTTCTAG | of V-A acrRNAVA1 |
| | CGACACAAACACAGCTTGCCAATAATACTTGTAAGGGGATTTTGTGCATT | sequence from NCBI database: |
| | TTTAACTCCAAATAGGGTGCAAGGCGTGGCGGTAGCTACTCCTGCCTTGC | NKHK01000012.1 |
| | CGTCGGTGACCATTGTCCTAATCGTATCAACTGGCCGt | |
| AcrRNAVA2 | ACTGTTTCTCCATTGTTTTTAAACCATGTCAATTGTCTAACAACTTTTTAA | DNA Fragment for amplification |
| | ATTTCTACTGTTTGTAGATACTGGCCGT | of V-A acrRNAVA2 |
| | | sequence from NCBI database: |
| | | CP011376.1 |
| AcrRNAVA3 | ACTGTTTCTCCATTGGTCGCATCACAGCAAATAGCAGTCTAACGACCTTTT | DNA Fragment for amplification |
| | AAATTTCTACTGTTTGTAGATTTCCTGTTGGGGATTATCAGCGGCTTATT | of V-A acrRNAVA3 |
| | GGTTATTTGCGCTTTGACAGATTTTGTGTGAAATATCCTGACAGCAAGGC | sequence from NCBI database: |
| | GGATTTTAACTGCTTTGAGCCGTCGGTGACCATTGTCCTAATCGTATCAA | CP011377.1 |
| | CTGGCCGT | |
| AcrRNAIE1 | ACTGTTTCTCCATCCGTGTTCCCCGCGTGTGCGGGGATGAACCGTGAGA | DNA Fragment for amplification |
| | GTATCCGTGACGTCCTGCACCCTTCTGATGCGAGTCATCAAAGCCCATGC | of acrRNAIE1 |
| | CCACTGGCGTTGGCGCGCCTGACCTCCTTGTTCTCCGGCTCGCCGGCGTC | sequence from NCBI database: |
| | GGTGACCATTGTCCTAATCGTATCAACTGGCCGT | CP011835.1 |
| | TACCCATGAGCACCATCATCGACCAGGACGGCGAGGAAATCGACTAACG | DNA Fragment for amplification |
| AcrRNAIC1 | CCACGGCGGTCAACCTCATGGACGATGAGATCCGCGAGGAACTCCACGC | of acrRNAIC1 |
| | CGAGATGGCACTCTGCACCGATCAGCAGTTCTTCGACGCCTACATCGAAA | sequence from NCBI database: |
| | GGCACTACGCCAAGTACGGCGAGGATTTCACCATCTGACAAGCACGGCG | QRXC01000024.1 |
| | GTCGCGCCCCGCGAGGGGGCGCGTGGATCGAAACACGACCATCACGGT | |
| | CCCACCCGGGCCGACACCGATCCAAGACGGGAGCATCCGACATGGGGC | |
| | ATCGTAACGGCAAACGCCTACGCCTCATACCATCACACCTGCCCGTCGGT | |
| | GACCATTGTCCTAATCGTATCAACTGGCCGT | |

### Example 1 Construction of Vectors for acrRNA's and Genes of Interest

### Plasmid Extractions

Plasmid DNA was extracted from an overnight culture of the according host strain with either the Plasmid Mini AX kit (A&A Biotechnology) for plasmids larger than 15 kb and the QIAprep Spin Miniprep Kit (Qiagen) for plasmids smaller than that. Concentrations of the DNA extract were determined by means of the Qubit. Fluorometer (Invitrogen) as instructed by the manufacturer.

TABLE 8
Vector construction
Expression vectors utilized for testing effect of acrRNAs

| | | | Expressed | |
|---|---|---|---|---|
| | | | gene of | |
| Alias | Backbone | Promoter | interest | Description |
| pHerd | pHerd30T | pBad | — | empty vector. NCBI database: EU603326.1 |
| 30T-ev | | | | |
| pRSO | pHerd30T | native | I-F | I-F-repeat like acrRNA773 identified on a phage genome |
| | | phage | acrRNA773 | (NCBI database: CP011110.1) expressed under control of |
| | | promoter | | the predicted wild type promoter |
| | | (pNative) | | |
| pSR1 | pHerd30T | native | I-F | I-F-repeat like acrRNA865 identified on a phage genome |
| | | phage | acrRNA865 | (NCBI database: NC_018012.1) expressed under control |
| | | promoter | | of the predicted wild type promoter |
| | | (pNative) | | |
| pSR2 | pHerd30T | pBad | I-F | I-F-repeat like acrRNA865 identified on a phage genome |
| | | | acrRNA865 | (NCBI database: NC_018012.1) expressed under control |
| | | | | of the L-ara. induced pBad promoter |
| pSR3 | pHerd30T | pBad | I-F repeat | Native I-F-repeat expressed under control of the L-ara. |
| | | | native to | induced pBad promoter |
| | | | PA14 | (seq: GTTCACTGCCGTATAGGCAGCTAAGAAA) |
| pSR4 | pHerd30T | pBad | ″synthetic″ | Exclusively the palindromic repeat identified in the |
| | | | acrRNA865 | phage genome under control of the L-ara. induced pBad |
| | | | | promoter |
| | | | | (seq: gttcactgccggataggcagccaaggaaatc) |
| pSR5 | pHerd30T | pBad | V-A | V-A-repeat like acrRNA1792 identified on a phage |
| | | | acrRNA1792 | genome (NCBI database: CP011377.1) expressed under |
| | | | | control of the L-ara. induced pBad promoter |
| pSR6 | pHerd30T | pBad | V-A | V-A-repeat like acrRNA1794 identified on a phage |
| | | | acrRNA1794 | genome (NCBI database: CP011376.1) expressed under |
| | | | | control of the L-ara. induced pBad promoter |
| pSR7 | pHerd30T | pBad | V-A | Native V-A-repeat expressed under control of the L-ara. |
| | | | repeat | induced pBad promoter. |
| | | | native to | (seq: GTCTAACGACCTTTTAAATTTCTACTGTTTGTAGAT) |
| | | | Moraxella | |
| | | | bovoculi | |
| pSR8 | pHerd30T | pBad | AcrRNAVA1 | V-A-repeat like acrRNAVA1 identified on a phage |
| | | | | genome (NCBI database: NKHK01000012.1) expressed |
| | | | | under control of the L-ara. induced pBad promoter |
| pSR9 | pHerd30T | pBad | AcrRNAVA2 | V-A-repeat like acrRNAVA2 identified on a phage |
| | | | | genome (NCBI database: CP011376.1) expressed under |
| | | | | control of the L-ara. induced pBad promoter |
| pSR10 | pHerd30T | pBad | AcrRNAVA3 | V-A-repeat like acrRNAVA3 identified on a phage |
| | | | | genome (NCBI database: CP011377.1) expressed under |
| | | | | control of the L-ara. induced pBad promoter |
| pSR11 | pHerd30T | pBad | AcrRNAVA4 | Native V-A-repeat acrRNAVA4 expressed under control |
| | | | | of the L-ara. induced pBad promoter. |
| | | | | (seq: GTCTAACGACCTTTTAAATTTCTACTGTTTGTAGAT) |
| pSR12 | pHerd30T | pBad | AcrRNAIE1 | I-E-repeat like acrRNAIE1 identified on a phage genome |
| | | | | (NCBI database: |
| | | | | CP011835.1) expressed under control of the L-ara. |
| | | | | induced pBad promoter |
| pSR13 | pHerd30T | pBad | AcrRNAIE2 | Native I-E-repeat acrRNAIE2 expressed under control of |
| | | | | the L-ara. induced pBad promoter. |
| | | | | (seq: GTGTTCCCCACGGGTGTGGGGATGAACC) |
| pSR14 | pHerd30T | pBad | AcrRNAIC5 | I-C-repeat like acrRNAIC1 identified on a phage genome |
| | | | | (NCBI database: |
| | | | | QRXC01000024.1) expressed under control of the L-ara. |
| | | | | induced pBad promoter |

Constructing of the expression vectors expressing genes of interest was done using USER® cloning (NEB, USA). DNA amplification for all inserts and backbones was done by using the Phusion U Hot Start DNA Polymerase (Thermo Scientific) as instructed by the provider. Melting temperatures of the primers were calculated with the help of the Tm calculator from ThermoFisher Scientific. PCR products were segregated after size via gel electrophoresis and the product with the correct size was subsequently purified from the gel using the QIAquick Gel Extraction Kit (Qiagen). The gel electrophoresis was performed on solidified (1% Biotechnology grade Agarose I; VWR International) 1× Modified Tris-Acetate EDTA (TAE) buffer, supplemented with 1 drop of 0.07% ethidium bromide/100 μl TAE buffer. Segregation of DNA has been conducted at 120V for 20 min in 1× TAE buffer. The bands were visualized with the help of the G:box F3 (Syngene) equipped with a UV transilluminator, controlled by Genesys v. 1.5 software (Syngene).

Assembly of the fragments (USER® assembly) was conducted with the help of the USER® enzyme (New England Biolabs) as recommended by the manufacturer. The ratio between backbone and insert DNA was chosen to be approximately 0.015 pmol to approximately 0.15 pmol, with the DNA of the smaller insert fragment exceeding the DNA of the backbone fragment 10-fold. The assembled product was subsequently transformed into chemically competent E. coli genehogs.

Positive clones were screened for by colony-PCRs using primers flanking the insert region. Colony PCRs were conducted by picking one single colony and its dilution in a PCR reaction mix including the PCRBIO HiFi Polymerase. The PCR reactions were prepared as recommended by PCRBIOSYSTEMS. The amplified products were sequenced to confirm correctness of the construct. All primers were ordered as oligonucleotides from from a commercial provider. Molecular design of DNA sequences as well as mapping of fragment sequences to reference sequences was done using the software SnapGene® 1.1.3 with default

### Example 2—Preparation of Strains with Expression Vectors Encoding acrRNA's and Genes of Interest

### Electro Competent Transformed P. Aeroguinosa Cells

Electrocompetent P. aeroguinosa cells were prepared by (i) streaking out the P. aeroguinosa cells on a selective medium; (ii) a single colony was picked and utilized to prepare an overnight culture of P. aeroguinosa. Cells were harvested (5000 g; 10 min; 4° C.), the supernatant removed, and the pellet was washed in the same volume of room-temperature 300 mM succrose twice. The cells were harvested again and subsequently diluted in 1/10 of the original culture volume. Glycerol was added to a final concentration of around 15%, aliquots à 100 μl were prepared, and finally frozen at −80° C.

For preparation of functional testing of acrRNAs, the pHerd30T plasmids with different candidate acrRNAs were electroporated into the different P. aeruginosa strains. Briefly, the electrocompent cells were carefully thawed when needed and incubated with around 2 μl (>500 ug) of DNA of interest for 30 min on ice. The cells were then transferred to a 2 mm electroporation cuvette and exposed to 2500 V. Recovery of the cells was conducted in 600 μl LB broth at 30° C. for 1h.

Chemically Competent Transformed E. coli Genehogs Cells

Chemically competent E. coli genehogs cells were prepared by (i) streaking out the E. coli genehogs on a selective medium; (ii) a single colony was picked and utilized to prepare an overnight culture of the E. coli genehogs, which (iii) was used to inoculate 100 mL LB Miller broth in an Ehrlenmeyer flasks to develop an OD600=0.02. The cultures were then grown up to an OD600=0.6. Cells were harvested (5000 g; 10 min; 4° C.), the supernatant removed, and the pellet was carefully suspended in 10 mL (0.1× of the original culture volume) of ice-cold 0.1M CaCl₂) (Sigma Aldrich). After 10 min of incubation on ice, the cells were harvested again (5000 g; 10 min; 4° C.), carefully dissolved in 4 mL (0.04× of the original culture) of ice cold 0.1M CaCl₂) and incubated on ice for 1h. Afterwards, 0.5 mL of ice-cold 80% glycerol was added, carefully mixed and aliquots à 50 μl competent cells were prepared. The aliquots were then frozen at −80° C. All steps from (iii) onwards were conducted on ice or on 4° C. and the cells were not vortexed at any point.

The chemically competent cells were carefully thawed on ice, DNA was added (<500 ng) and incubated for 20 to 30 min. The cells were then exposed to 42° C. for 45s and subsequently placed on ice for 2 min. 500 μl LB Miller broth was added and the cells were recovered for 1h at 30° C. and 250 rpm. The transformed cells were then spread out on solid media with the according antibiotic resistances (100 μl on one plate, rest of the cells on another plate).

### Example 3—Cultivation of Cells Expressing of acrRNA's and Genes of Interest

Samples of cells from example 2 were grown on/in LB media. Overnight cultures were grown in 5 mL LB broth at 30° C. and 350 rpm for 15-16h. LB agar plates have been prepared with 1.5% agar and bacterial growth on the solid media was conducted at 30° C. as well. Antibiotics and inducers were added to the according growth media if necessary.

### Example 4—Screening for and Designing acrRNAs

Screen for acrRNAs

BLAST searches using known CRISPR repeats (specific for each CRISPR-Cas system subtype/variant) across NCBI public prokaryotic genome sequence databases were carried out (95% identity and sequence coverage). Sequences matching a known CRISPR repeat were selected as potential acrRNAs, except for those within a distance of 100 bp, which are disregarded in order to avoid false-positive detection of true CRISPR arrays. Potential acrRNAs were screened for their association to phage/MGE sequences with virsorter and PHASTER (integrated or extrachromosomal). Candidates present on an MGE genome were selected for being likely true acrRNAs. The identification of potential promoter sequence regions in front of the putative acrRNA were predicted via pBrom. When possible, the presence of host CRISPR-Cas targeting was assessed and stable coexistence of the targeted MGE inside the host cell was considered is a good indicator of CRISPR-Cas inhibition. (Watters, K. E. et al. (2020) ‘Potent CRISPR-Cas9 inhibitors from Staphylococcus genomes’, PNAS; Rauch, B. J. et al. (2017) ‘Inhibition of CRISPR-Cas9 with Bacteriophage Proteins’, Cell, 168(1-2), pp. 150-158; Marino, N. D. et al. (2018) ‘Discovery of widespread type I and type V CRISPR-Cas inhibitors’, Science, 362(6411), pp. 240-242; Borges, A. L., Davidson, A. R. and Bondy-Denomy, J. (2018) ‘The Discovery, Mechanisms, and Evolutionary Impact of Anti-CRISPRs’, Annual review of virology, 4(1), pp. 37-59).

Design of acrRNAs

AcrRNAs identified on phage genomes were ordered as gene fragments from a commercial provider (IDT) and cloned into pHerd30t under (i) the native promoter, and (ii) under the L-arabinose inducible pBad promoter. The repeat sequence native to the corresponding system was designed and cloned as a “synthetic” acrRNA under expression regulation of pBad.

### Example 5—Testing Effect of acrRNAs

### Phage Propagation

Pseudomonas phages DMS3m, and JBD30 derivatives were propagated on PA14 ΔCRISPR, PA scm4386 or PAO1 WT. Pseudomonas phages were stored at 4° C. in SM-buffer over chloroform.

### Phage Spotting Assay

The functionality of the acrRNAs was assessed through phage spotting assays. Bacterial lawns of the model organisms (see table 4) were challenged with a CRISPR-Cas targeted phage (DMS3m or JBD30 passed through the respective non-targeting strain). These tests evaluated the replication of CRISPR-targeted phages DMS3m and JBD30 in bacterial lawns expressing the acrRNA from the vector pHerd30T relative to the empty vector control.

Briefly, 150 μL of bacterial overnight cultures were combined with 4 mL of molten top agar (0.7%) supplemented with 10 mM MgSO4 and the appropriate inducers. The mix was poured onto LB agar (1.5%) plates containing the inducers and antibiotics, 10 mM MgSO4 and 0.3% w/v arabinose (induction). Phage dilutions 2.4 μL of ten-fold serial dilutions of the respective phage lysates were spotted onto the plate surface containing the bacterial lawn in the top agar. The plates were incubated at 30° C. ON and pictures were taken the next day.

### Results

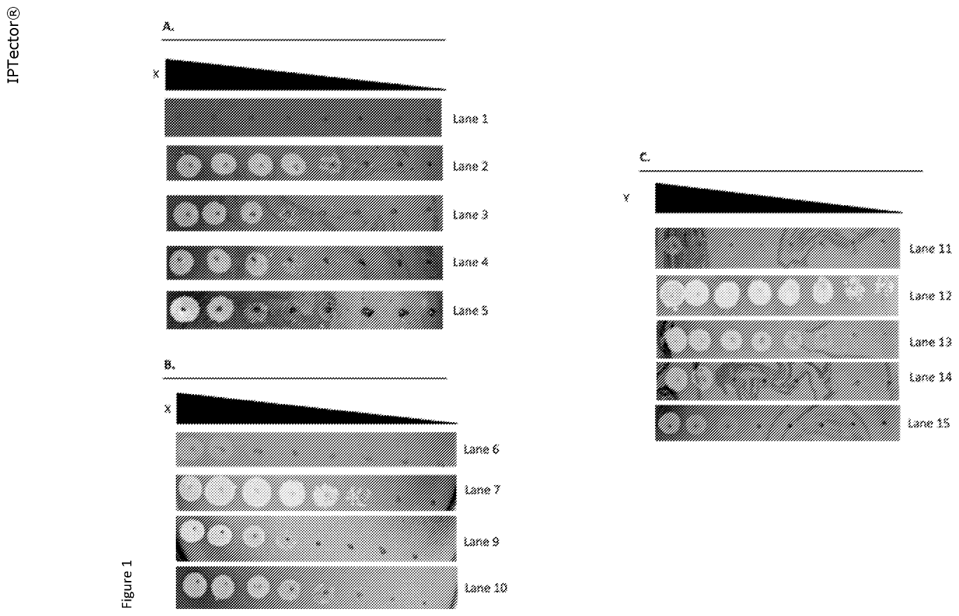

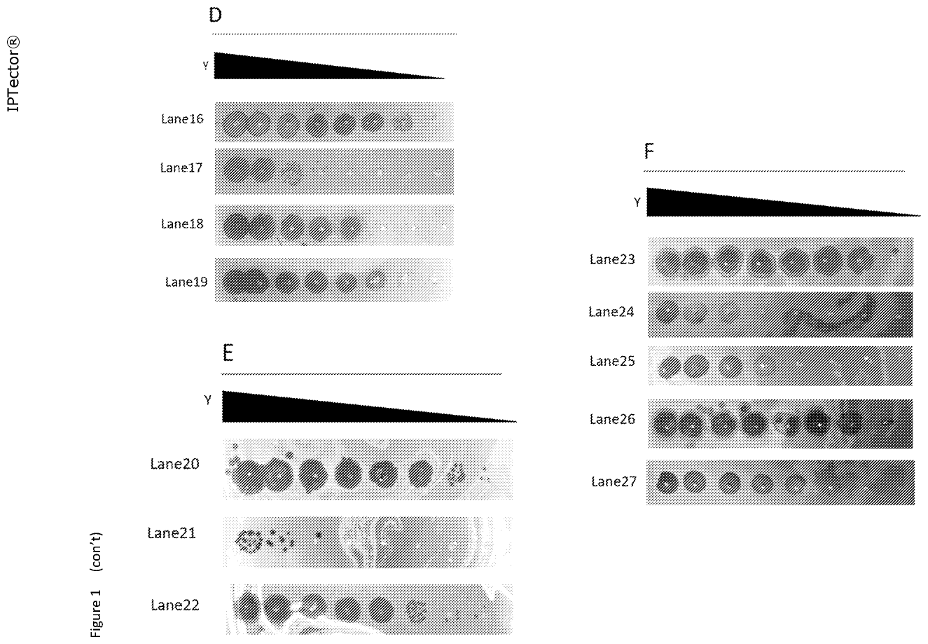

The results of the phage spotting assay are displayed in FIG. 1, where lane 1 to 27 shows bacterial lawns on which the phage serial dilution was spotted. FIG. 1A shows the assaying of synthetic acrRNAs designed by the present inventors inhibiting the wild type CRISPR-Cas I-F system in P. aeruginosa PA14. FIG. 1B shows the assaying of natural acrRNAs isolated by the present inventors inhibiting the wild type CRISPR-Cas I-F system in P. aeruginosa PA14. FIG. 1C shows the assaying of synthetic and isolated natural acrRNAs inhibiting MbCpf1 activity in PAO1. FIG. 1D shows the assaying of natural and synthetic I-E acrRNAs designed by the inventors inhibiting the wild type CRISPR-Cas I-E system in P. aeruginosa PA scm 4386. FIG. 1E shows the assaying of synthetic I-C acrRNAs designed by the inventors inhibiting the heterologous CRISPR-Cas I-C LL77 system in PAO1. FIG. 1F shows the assaying of more natural and synthetic V-A acrRNAs designed by the inventors inhibiting the heterologous CRISPR-Cas V-A (Mb) system in PAO1. In 1A-F, X Indicates a 10-fold serial dilution of phage DMS3m and Y indicates a 10-fold serial dilution of phage JBD30.

Lane 1—Bacterial Lawn of RPR145 Harboring pHerd30T-Ev Challenged by Phage DMS3m.

This lane shows a bacterial lawn of PA14 with an active CRISPR-Cas type I-F system, targeting the phage DMS3m. Phage replication is inhibited.

Lane 2—Bacterial Lawn of RPR146 Harboring pHerd30T-Ev Challenged by Phage DMS3m.

This lane shows a bacterial lawn of PA14 deletion strain CRISPR-Cas type I-F ΔCRISPR1, not targeting the phage DMS3m. Phage replication is not prohibited by the CRISPR-Cas system.

Lane 3—Bacterial Lawn of RPR145 Harboring pSR2 Challenged by Phage DMS3m.

This lane shows a bacterial lawn of PA14 with an active CRISPR-Cas type I-F system, targeting the phage DMS3m. The expression of the acrRNA865 (SEQ ID NO: 1208) under the pBad promoter inhibits the I-F CRISPR-Cas system and thereby enables phage replication.

Lane 4—Bacterial Lawn of RPR145 Harboring pSR3 Challenged by Phage DMS3m.

This lane shows a bacterial lawn of PA14 with an active CRISPR-Cas type I-F system, targeting the phage DMS3m. The expression of the native I-F PA14 repeat sequence (SEQ ID NO: 1211) under the pBad promoter inhibits the I-F CRISPR-Cas system and thereby enables phage replication.

Lane 5—Bacterial Lawn of RPR145 Harboring pSR4 Challenged by Phage DMS3m.

This lane shows a bacterial lawn of PA14 with an active CRISPR-Cas type I-F system, targeting the phage DMS3m. The expression of the “synthetic” acrRNA865 (SEQ ID NO: 1208) under the pBad promoter inhibits the I-F CRISPR-Cas system and thereby enables phage replication.

Lane 6—Bacterial Lawn of RPR145 Harboring pHerd30T-Ev Challenged by Phage DMS3m.

This lane shows a bacterial lawn of PA14 deletion strain CRISPR-Cas type I-F ΔCRISPR1, not targeting the phage DMS3m. Phage replication is not prohibited by the CRISPR-Cas system.

Lane 7—Bacterial Lawn of RPR146 Harboring pHerd30T-Ev Challenged by Phage DMS3m.

This lane shows a bacterial lawn of PA14 with an active CRISPR-Cas type I-F system, targeting the phage DMS3m. The expression of the acrRNA865 (SEQ ID NO: 1213) under the pBad promoter inhibits the I-F CRISPR-Cas system and thereby enables phage replication.

Lane 9—Bacterial Lawn of RPR145 Harboring pSRO Challenged by Phage DMS3m.

This lane shows a bacterial lawn of PA14 with an active CRISPR-Cas type I-F system, targeting the phage DMS3m. The expression of the acrRNA773 (SEQ ID NO: 1207) under the native promoter inhibits the I-F CRISPR-Cas system and thereby enables phage replication.

Lane 10—Bacterial Lawn of RPR145 Harboring pSR1 Challenged by Phage DMS3m.

This lane shows a bacterial lawn of PA14 with an active CRISPR-Cas type I-F system, targeting the phage DMS3m. The expression of the acrRNA865 (SEQ ID NO: 1208) under the native promoter inhibits the I-F CRISPR-Cas system and thereby enables phage replication.

Lane 11: Bacterial Lawn of RPR212 Harboring pHerd30T-Ev Challenged by Phage JBD30.

This lane shows a bacterial lawn of PAO1 genetically engineered with MbCpf1 and a crRNA, targeting the phage JBD30. Phage replication is inhibited.

Lane 12: Bacterial Lawn of RPR213 Harboring pHerd30T-Ev Challenged by Phage JBD30.

This lane shows a bacterial lawn of PAO1 genetically engineered with MbCpf1, lacking a crRNA, not targeting the phage JBD30. Phage replication is not prohibited.

Lane 13: Bacterial Lawn of RPR212 Harboring pSR7 Challenged by Phage JBD30.

This lane shows a bacterial lawn of PAO1 genetically engineered with MbCpf1 and a crRNA, targeting the phage JBD30. The expression of the native V-A repeat (SEQ ID NO: 1212) under the pBad promoter inhibits the targeting by MbCpf1 and thereby enables phage replication.

Lane 14: Bacterial Lawn of RPR212 Harboring pSR5 Challenged by Phage JBD30.

This lane shows a bacterial lawn of PAO1 genetically engineered with MbCpf1 and a crRNA, targeting the phage JBD30. The expression of the acrRNA1792 (SEQ ID NO: 1209) under the pBad promoter inhibits the targeting by MbCpf1 and thereby enables phage replication.

Lane 15: Bacterial Lawn of RPR212 Harboring pSR6 Challenged by Phage JBD30.

This lane shows a bacterial lawn of PAO1 genetically engineered with MbCpf1 and a crRNA, targeting the phage JBD30. The expression of the acrRNA1794 (SEQ ID NO: 1210) under the pBad promoter inhibits the targeting by MbCpf1 and thereby enables phage replication.

Lane 16: Bacterial Lawn of SC116 Harboring pHerd30T-Ev Challenged by Phage JBD30.

This lane shows a bacterial lawn of PAscm4386 with an inactive CRISPR-Cas I-E (Cas3 knockout). The phage can replicate.

Lane 17: Bacterial Lawn of SC115 Harboring pHerd30T-Ev Challenged by Phage JBD30.

This lane shows a bacterial lawn of PAscm4386 with an active CRISPR-Cas I-E, targeting the phage JBD30. The phage cannot replicate.

Lane 18: Bacterial Lawn of SC115 Harboring pSR12 Challenged by Phage JBD30.

This lane shows a bacterial lawn of PAscm4386 with an active CRISPR-Cas I-E, targeting the phage JBD30. The expression of the acrRNAIE1 (SEQ ID NO: 1201) under the pBad promoter inhibits the targeting and thereby enables phage replication.

Lane 19: Bacterial Lawn of SC115 Harboring pSR13 Challenged by Phage JBD30.

This lane shows a bacterial lawn of PAscm4386 with an active CRISPR-Cas I-E, targeting the phage JBD30. The expression of the acrRNAIE2 (SEQ ID NO:1202) under the pBad promoter inhibits the targeting and thereby enables phage replication.

Lane 20: Bacterial Lawn of RPR147 Harboring pHerd30T-Ev Challenged by Phage JBD30.

This lane shows a bacterial lawn of wild type PAO1 without CRISPR-Cas. The phage can replicate.

Lane 21: Bacterial Lawn of RPR148 Harboring pHerd30T-Ev Challenged by Phage JBD30.

This lane shows a bacterial lawn of PAO1 with a heterologous I-C CRISPR-Cas, targeting the phage JBD30. The phage cannot replicate.

Lane 22: Bacterial Lawn of RPR148 Harboring pSR14 Challenged by Phage JBD30.

This lane shows a bacterial lawn of shows a bacterial lawn of PAO1 with a heterologous I-C CRISPR-Cas, targeting the phage JBD30. The expression of the acrRNAIC1 (SEQ ID NO: 1203) under the pBad promoter inhibits the targeting and thereby enables phage replication.

Lane 23: Bacterial Lawn of RPR213 Harboring pHerd30T-Ev Challenged by Phage JBD30.

This lane shows a bacterial lawn of PAO1 genetically engineered with MbCpf1, lacking a crRNA, not targeting the phage JBD30. The phage can replicate.

Lane 24: Bacterial Lawn of RPR212 Harboring pHerd30T-Ev Challenged by Phage JBD30.

This lane shows a bacterial lawn of PAO1 genetically engineered with MbCpf1 and a crRNA, targeting the phage JBD30. Phage replication is inhibited.

Lane 25: Bacterial Lawn of RPR212 Harboring pSR8 Challenged by Phage JBD30.

This lane shows a bacterial lawn of PAO1 genetically engineered with MbCpf1 and a crRNA, targeting the phage JBD30. The expression of the native acrRNAVA1 (SEQ ID NO: 1204) under the pBad promoter inhibits the targeting by MbCpf1 and thereby enables phage replication.

Lane 26: Bacterial Lawn of RPR212 Harboring pSR9 Challenged by Phage JBD30.

This lane shows a bacterial lawn of PAO1 genetically engineered with MbCpf1 and a crRNA, targeting the phage JBD30. The expression of the native acrRNAVA2 (SEQ ID NO: 1205) under the pBad promoter inhibits the targeting by MbCpf1 and thereby enables phage replication.

Lane 27: Bacterial Lawn of RPR212 Harboring pSR10 Challenged by Phage JBD30.

5 This lane shows a bacterial lawn of PAO1 genetically engineered with MbCpf1 and a crRNA, targeting the phage JBD30. The expression of the native acrRNAVA3 (SEQ ID NO: 1206) under the pBad promoter inhibits the targeting by MbCpf1 and thereby enables phage replication.

1-70. (canceled)

71. A method of modulating an activity of a Cas-effector on a target polynucleotide comprising contacting the Cas-effector with an inhibitor component, wherein the inhibitor component comprises an anti-CRISPR ribonucleotide sequence (acrRNA) capable of inhibiting the Cas-effector from (i) associating with a target nucleotide sequence; and/or (ii) associating with a CRISPR guide RNA, and thereby inhibiting the Cas-effector from forming an active RNA-guided Cas-effector protein complex, wherein the Cas-effector is selected from a type I, type III, type IV, type V and/or type VI Cas-effector and wherein optionally the Cas-effector comprises a Cas3, Cas5, Cas6, Cas7, Cas8, Cas10, DinG, RecD, LS, Cas11, Cas12, Cas12f, Cas13 and/or Cas14 protein complex.

72. The method of claim 71, wherein the protein complex comprises an amino acid sequence which is at least 70% identical to any one of SEQ ID NOs: 1146 to 1184.

73. The method of claim 71, wherein the CRISPR guide RNA is a CRISPR RNA (crRNA), includes a trans-activating CRISPR RNA (tracrRNA); and/or is a fusion of a crRNA and a tracrRNA (crRNA-tracrRNA fusion).

74. The method of claim 71, wherein the acrRNA comprises a ribonucleotide sequence having at least 70% identity to a sequence of the structural moiety of the CRISPR guide RNA, which binds to one or more components of the Cas-effector, and wherein the arcRNA lacks a spacer sequence of the guide RNA recognizing the target nucleotide sequence.

75. The method of claim 71, wherein the acrRNA (i) comprises a sequence that is at least 70% identical to any one of SEQ ID NO: 10 to 13, or 1201 to 1213, or
(ii) comprises at least one repeat sequence of the structural moiety of the CRISPR guide RNA, which binds to the one or more components of the Cas-effector, wherein optionally said repeat sequence is palindromic, semi-palindromic and/or cognate, wherein optionally said repeat sequence is selected from a type I, type III, type IV, type V, type VI CRISPR-Cas system repeat sequence, and/or wherein optionally said repeat sequence has at least 70% identity to a repeat sequence comprised in any one of SEQ ID NO: 14 to 929; or
(iii) comprises a moiety hybridizing to the CRISPR guide RNA and thereby inhibits the CRISPR guide RNA from associating with the Cas-effector, wherein optionally said moiety is a ribonucleotide sequence which is an anti-repeat sequence complementary to a repeat sequence of the CRISPR guide RNA, wherein optionally the anti-repeat is at least 70% identical to the sequence complementary to the repeat sequence comprised in SEQ ID NO: 14 to 929.

76. The method of claim 73, wherein
(i) the crRNA is a type I, type III, type IV, type V and/or type VI CRISPR-Cas system crRNA,
(ii) the tracrRNA is a type II or type V CRISPR-Cas system tracrRNA, optionally having at least 70% identity to the tracrRNA comprised in SEQ ID NO: 930 to 1145; and/or
(iii) the crRNA-tracrRNA fusion is a type V CRISPR-Cas system crRNA-tracrRNA fusion.

77. The method of claim 71, wherein contacting the Cas-effector with the inhibitor component is performed in vivo in a living cell.

78. The method of claim 77, wherein the cell is a eukaryotic cell, animal cell, mammalian cell, human cell, blood or an induced pluripotent stem cell, prokaryotic (bacteria or archaea) cell, plant cell, insect cell, or fungal cell.

79. The method of claim 77, wherein the cell comprises a transgene encoding the acrRNA, wherein optionally
(i) the cell comprises a self-replicating genetic element comprising the transgene encoding the acrRNA;
(ii) the transgene is operably linked to a heterologous, optionally constitutive, regulatory expression element, which optionally is controllable in response to a condition selected from the group consisting of temperature, presence or absence of a molecule/ligand, activation or suppression of an endogenous gene, light, sound, cell cycle, organism phase, tissue, cell type or environmental stress.

80. The method of claim 77, wherein the acrRNA is fed to the cell exogenously, optionally by contacting the cell with a delivery vehicle comprising the acrRNA.

81. The method of claim 80, wherein the delivery vehicle comprises a liposome, nanoparticle or a phage particle.

82. The method of claim 71, wherein contacting the Cas-effector with the inhibitor component is performed ex vivo.

83. The method of claim 72, wherein contacting the Cas-effector with the inhibitor component is performed ex vivo in a medium comprising an extract of a cell contacted with the Cas-effector prior to extraction, and providing for cell-free transcription-translation protein synthesis in the medium.

84. The method of claim 72, wherein contacting the Cas-effector with the inhibitor component is performed in a medium comprising an extract of a cell contacted with the Cas-effector prior to extraction and comprising the Cas-effector.

85. The method of claim 72, wherein contacting the Cas-effector with the inhibitor component is performed in a medium providing for DNA or RNA synthesis.

86. An acrRNA capable of inhibiting a Cas-effector from (i) associating with a target nucleotide sequence; and/or (ii) associating with a CRISPR guide RNA, and thereby inhibiting the Cas-effector from forming an active RNA-guided Cas-effector complex, wherein the Cas-effector is selected from a type I, type III, type IV, type V and/or type VI Cas-effector and optionally comprises a Cas3, Cas5, Cas6, Cas7, Cas8, Cas10, DinG, RecD, LS, Cas11, Cas12, Cas12f, Cas13 and/or Cas14 protein complex, optionally comprising an amino acid sequence which is at least 70% identical to SEQ ID NO: 1146 to 1184, and wherein optionally the acrRNA comprise:
(a) a sequence that is at least 70% identical to any one of SEQ ID NO: 10 to 13, or 1201 to 1213;
(b) comprises at least one repeat sequence of the structural moiety of the CRISPR guide RNA, which binds to the one or more components of the Cas-effector, said repeat sequence is palindromic, semi-palindromic and/or cognate, said repeat sequence is selected from a type I, type III, type IV, type V, type VI CRISPR-Cas system repeat sequence.
(c) comprises a moiety hybridizing to the CRISPR guide RNA and thereby inhibits the CRISPR guide RNA from associating with the Cas-effector, said moiety is a ribonucleotide sequence which is an anti-repeat sequence complementary to a repeat sequence of the CRISPR guide RNA, The acrRNA of claim 26, wherein the anti-repeat is at least 70% identical to the sequence complementary to the repeat sequence comprised in SEQ ID NO: 14 to 929.

87. A delivery vehicle comprising the acrRNA of claim 86, said delivery vehicle optionally comprising a liposome, nanoparticle or a phage particle.

88. A genetically modified host cell comprising a gene encoding the acrRNA of claim 86.

89. A composition comprising the acrRNA of claim 86.
